(19) United States
(12) Patent Application Publication
(10) Pub. No.: US 2026/0022314 A1
(43) Pub. Date: Jan. 22, 2026

(54) SACCHAROMYCES EUBAYANUS MALTOTRIOSE TRANSPORTERS AND USES THEREOF

(71) Applicant: Wisconsin Alumni Research Foundation, Madison, WI (US)

(72) Inventors: Christopher Hittinger, Madison, WI (US); Johnathan Crandall, Madison, WI (US)

(21) Appl. No.: 19/274,171

(22) Filed: Jul. 18, 2025

Related U.S. Application Data
(60) Provisional application No. 63/673,398, filed on Jul. 19, 2024.

(51) Int. Cl. C12C 12/00; C07K 14/395; C12N 1/18; C12N 15/81; C12R 1/85

(52) U.S. Cl. CPC C12C 12/006; C07K 14/395; C12N 1/18; C12N 15/81; C12R 2001/85

(57) ABSTRACT

The present invention provides engineered Saccharomyces eubayanus MalT4 proteins that have maltotriose transport activity. Also provided are polypeptides, constructs, and vectors encoding the engineered MalT4 proteins, as well as S. eubayanus yeast cells that express the engineered MalT4 proteins and methods of using these cells to make a fermentation product, such as a lager.

### CROSS-REFERENCE TO RELATED APPLICATIONS

This patent application claims priority to U.S. Provisional Application No. 63/673,398, filed on Jul. 19, 2024, the contents of which are incorporated by reference in their entireties.

### STATEMENT REGARDING FEDERALLY SPONSORED RESEARCH

This invention was made with government support under 2110403 awarded by the National Science Foundation and under DE-SC0018409 awarded by the US Department of Energy and under NI20HFPXXXXXG057 and NI24HFPXXXXXG031 awarded by the USDA/NIFA. The government has certain rights in the invention.

### SEQUENCE LISTING STATEMENT

This application includes a sequence listing in XML format titled “960296_04706_ST26.xml”, which is 186,275 bytes in size and was created on Apr. 21, 2025. The sequence listing is electronically submitted with this application via Patent Center and is incorporated herein by reference in its entirety.

### BACKGROUND

A key consideration for any new brewing strain is its ability to rapidly and completely use brewing-related sugars, the most important being maltose, followed by maltotriose and glucose. Of these sugars, maltotriose is by far the most difficult to ferment, and it comprises around 20% of fermentable sugars in wort.

Recently, there has been great interest in using the wild yeast Saccharomyces eubayanus, the cold-tolerant parent of the industrial lager-brewing yeast hybrids, to produce lager-style beers, which constitute the vast majority of the international market share. However, no wild strains of S. eubayanus can ferment maltotriose, as most lack proteins that can transport this sugar into the cell. Poor utilization of maltotriose by brewing strains leads to large amounts of unconsumed sugar, lower amounts of ethanol, and a cloying flavor that is regarded as undesirable in most beer styles. Accordingly, there is a need in the art for new molecular tools that can be used to confer maltotriose utilization to yeast.

### SUMMARY

In a first aspect, the present invention provides engineered MalT4 proteins that have maltotriose transport activity. The engineered MalT4 proteins comprise the amino acid sequence of SEQ ID NO: 1 with amino acid substitutions in 3-7 positions selected from residues 468, 503, 504, 505, 508, 512, and 379 of SEQ ID NO: 1, wherein: (a) the substitution at residue 468 is S468F; (b) the substitution at residue 503 is I503M; (c) the substitution at residue 504 is G504A; (d) the substitution at residue 505 is selected from N505C, N505S, and N505G; (e) the substitution at residue 508 is V508T; (f) the substitution at residue 512 is selected from I512T and I512V; and/or (g) the substitution at residue 379 is S379C.

In a second aspect, the present invention provides polynucleotides encoding an engineered MalT4 proteins disclosed herein.

In a third aspect, the present invention provides constructs comprising a promoter operably linked to a polynucleotide disclosed herein.

In a fourth aspect, the present invention provides vectors comprising a polynucleotide or construct disclosed herein.

In a fifth aspect, the present invention provides Saccharomyces eubayanus yeast cells comprising an engineered MalT4 protein, polynucleotide, construct, or vector described herein.

In a sixth aspect, the present invention provides methods for making a fermentation product. The methods comprise culturing yeast cell described herein with a fermentable substrate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B show the architecture of a chimeric neofunctionalized α-glucoside transporter. FIG. 1A: A structural model of the chimeric transporter MalT434 is shown from the side and top views, with different shades of gray color demarking regions contributed by different parental proteins. The top view is orientated looking down the transport channel. MalT3 side chains are drawn for the 11 substitutions between MalT4 and MalT434. The asterisk label marks the position of the three substitutions on a helical face that bounds the transport channel. FIG. 1B: Schematic of mutations. The 11 mutations between MalT4 and MalT434 (i.e., at residues 468, 503, 504, 505, 508, 512, 522, 534, 536, 538, and 540) are drawn as side chains along the cartoon secondary structure of the protein, with loops that connect transmembrane helices truncated for clarity. Polar hydrogens are shown. Asterisks mark the amino acids that face the transport channel. The amino acids depicted below the structure are the amino acids present at the 11 differing residues in the wild-type MalT4 protein of SEQ ID NO: 1 and the chimeric MalT434 protein of SEQ ID NO: 2.

FIG. 2 shows that high-order intramolecular interactions are required to evolve a novel function in chimeric α-glucoside transporters. Points and bars show mean+/−SEM of normalized growth on maltotriose (AUC, area under the curve) of strains expressing chimeric transporters or wild-type MalT4 (top row). Filled circles denote growth significantly greater than the negative control (p<0.01, Mann-Whitney U test with Benjamini-Hochberg correction). The architecture of each tested transporter is depicted as a cartoon on the y-axis, where rounded rectangles represent each of the twelve transmembrane helices and circles represent the intracellular ICH domain that links the N- and C-terminal six-helix bundles; regions are colored by parental protein identity. In almost every case, the N- and C-terminal intracellular regions have the same parental protein identity as the neighboring transmembrane helix and are omitted for clarity; the two exceptions are depicted. Inverted arrows and brackets indicate the location and identity of protein regions underlying the largest detected intramolecular interaction.

FIG. 3 demonstrates that numerous substitutions are required to evolve a novel function in a maltose transporter. Points and bars show mean+/−SEM of normalized growth on maltotriose (AUC, area under the curve) of strains expressing MalT4 variants. The genotype of each protein at the 11 residues (i.e., residues 468, 503, 504, 505, 508, 512, 522, 534, 536, 538, and 540) that differ between MalT4 (top row, dark gray) and MalT434 (second from top row, light gray) is depicted on the Y-axis. Thus, the depicted amino acids are the amino acids present at these 11 residues in the MalT4 proteins of SEQ ID NOs: 1-16, numbered from top to bottom. Filled circles denote growth significantly greater than the negative control (p<0.01, Mann-Whitney U test with Benjamini-Hochberg correction). The bar chart shows rescaled BLOSUM similarity between the MalT4 and MalT3 residue at that site, with a higher bar indicating a more conservative substitution. Horizontal dotted lines in the protein haplotype grid separate related groups of genotypes. The vertical dotted line demarcates the substitutions that are sufficient (left) to impart novel function to MalT4 and those that are insufficient (right).

FIGS. 4A-4B demonstrate that a single amino acid underlies a large epistatic effect. FIG. 4A: Structural model of MalT434 with helices colored in different shades of gray according to parental protein. Side chains are drawn for amino acids on transmembrane helices 7, 11, and 12 that are polymorphic between MalT3 and MalT4, and those that are proximal to or project into the transport channel are labeled. FIG. 4B: Points and bars show mean+/−SEM of normalized growth on maltotriose (AUC, area under the curve) of strains expressing transporter variants. Filled circles denote growth significantly greater than the negative control (p<0.01, Mann-Whitney U test with Benjamini-Hochberg correction). Colors indicate parental protein identity at transmembrane helices 10 and 11 (filled rectangular ovals) and residues 378 and 379 in transmembrane helix 7.

FIGS. 5A-5B demonstrate that physicochemical requirements constrain the evolution of novel function. FIG. 5A: Points and bars show mean+/−SEM of normalized growth on maltotriose (AUC, area under the curve) of strains expressing MalT4 variants. The x-axis shows the amino acid identity at position 505; all variants share F468, M503, A504, T508, and T512. Filled circles denote growth significantly greater than the negative control (p<0.01, Mann-Whitney U test with Benjamini-Hochberg correction). FIG. 5B: Correlations between growth and properties of the amino acid variant at position 505. Growth is plotted as in (A) against physicochemical property or overall similarity to the wild-type residue at position 505, asparagine. Lines and shaded ranges show regressions and 95% confidence intervals for significant (p<0.05) regressions for all data (black) or after removing observations for C505 (gray). Dotted lines show regressions that are not statistically significant. Inset text shows Kendall's T; ***p<10⁻⁶, **p<10⁻⁴, *p<0.05.

FIGS. 6A-6B show that the high-specificity maltose transporters are evolutionarily derived and restricted to a subset of Saccharomycesles. FIG. 6A: Unrooted consensus phylogeny of the α-glucoside transporter clade from 332 budding yeast genomes. Agt1-like and Mal31-like proteins from all Saccharomycesles are colored. Bootstrap support is shown for two splits leading to the Saccharomycesles. FIG. 6B: Rooted consensus tree of the clade containing Saccharomycetales α-glucoside transporters. Branches are colored as in (a) with the inclusion of a well-supported clade of Brettanomyces Agt1-like proteins that nests within the Saccharomycesles; the Saccharomyces-specific Mph2/3 clade is also indicated. Circles denote branches with >90% bootstrap support. Colored bars outside the tree show genus-level taxonomic assignment, and the inset circular tree shows the Saccharomycotina species phylogeny (X.-X. Shen et al. 2018) with those genera colored; Zygo/Torulaspora represents Zygosaccharomyces, Zygotorulaspora, and Torulaspora. The rooted maximum-likelihood tree can be found in FIG. 13. The full MFS phylogeny can be found in FIG. 12. Scale bars indicate the number of substitutions per site.

FIGS. 7A-7C demonstrate that species with Agt proteins grow on Agt1-specific substrates. FIG. 7A: Time-calibrated phylogeny of 332 Saccharomycotina species (X.-X. Shen et al. 2018) with branches colored (key in FIG. 7C) by taxonomic order (Groenewald et al. 2023). Heatmaps around the tree show growth (normalized area under the curve) on α-glucosides: methyl-α-glucoside (inner ring), trehalose (middle ring), and maltotriose (outer ring). Gray boxes denote no growth above background; white boxes represent unsampled species. The bar chart shows the number of proteins in the α-glucoside transporter clade for each genome. FIG. 7B: Generalist Agt content of Saccharomycesles genomes is not representative. Density plots show distributions of the number of Agt-clade proteins per genome for Saccharomycesles species (dark gray) and species from all other orders (light gray). FIG. 7C: Scatterplots of Agt-clade transporter count versus growth on each α-glucoside. Each species is represented by a point, colored by taxonomic order. Lines and shaded regions are loess-smoothed regressions of the untransformed data; inset p-values are from phylogenetically corrected regressions (PGLSs).

FIGS. 8A-8C show the structural similarity of wild-type and chimeric S. eubayanus transporters. FIG. 8A: Structural models for wild-type MalT4 (teal) and MalT3 (orange), and the chimeric MalT434 (gray) are shown with intracellular N- and C-terminal domains hidden for clarity. FIG. 8B: Structural overlay of MalT3, MalT4, and MalT434. The structures are superimposable with mean and SD pairwise RMSD=0.955±0.107 Å. FIG. 8C: Structural overlay of transmembrane helices 10, 11, and 12 from MalT3 and MalT4, corresponding to the recombinant region in MalT434 (RMSD=0.909 Å).

FIGS. 9A-9B show amino acid differences between MalT3 and MalT4 along the key transmembrane helix 7. FIG. 9A: The structural models of MalT4 (dark gray) and Malt3 (light gray) are superimposed; transmembrane helix 7 is shown in full color, while all other transmembrane helices and the ICH domain are shown with transparency, and the intracellular N- and C-terminal domains are hidden. Side chains are drawn at the sites where MalT4 and MalT3 differ along TMH 7, with residue numbers labeled. FIG. 9B: Detail of the proximity between position 379 on TMH 7 and sites on TMH 11. Helices are shown from the structural model of the chimeric transporter MalT434 and colored according to parental protein (TMH 7, MalT4, dark gray; TMH 11, MalT3, light gray). Dotted lines and labels show the distance between residues 379 and 505, 508, and 512 (bottom to top, respectively), in angstroms.

FIG. 10 shows that the effect of residue C505 on maltotriose transport is due to its unique properties. Scatterplots show amino acids in principal component space based on dimensionality reduction of side chain properties. Colors show growth on maltotriose (AUC, area under the curve) of strains expressing MalT4 with the given amino acid at position 505, in the context of all other necessary mutations for maltotriose transport, as shown in FIG. 5. Amino acids in gray were not measured.

FIG. 11 shows further evidence for nuanced and context-specific biochemical requirements at key sites. Points and bars show mean+/−SEM of normalized growth on maltotriose (AUC, area under the curve) of strains expressing MalT4 variants, which each contain all other necessary mutations for maltotriose transport. The amino acid identity at position 379 (top panel) or 512 (bottom panel) is shown on the x-axis. Filled circles denote growth significantly greater than the negative control (p<0.01, Mann-Whitney U test with Benjamini-Hochberg correction).

FIGS. 12A-12B show unrooted (FIG. 12A) and midpoint-rooted (FIG. 12B) maximum-likelihood phylogeny of 8,403 sugar porters from 332 budding yeast genomes. An outgroup clade containing non-sugar porter Major Facilitator Superfamily proteins is hidden for clarity; rooting the tree to this outgroup recovers the same topology. Major clades containing at least one S. cerevisiae protein are colored according to the substrate(s) of the S. cerevisiae homolog: α-glucosides (red and blue), hexoses/monosaccharides (light green), glucose sensors (dark green), glycerol/sugar alcohols (gray), and undetermined (orange). The high-specificity maltose transporter clade, which contains the Saccharomyces-specific Mph2/3 proteins, is shown in blue. The branches drawn in red and blue correspond to the proteins in the α-glucoside transporter clade shown in FIG. 6.

FIG. 13 shows the maximum-likelihood phylogeny of the clade containing α-glucoside transporters, rooted to a divergent outgroup. Agt1- and Mal31-like transporters from Saccharomycetales are colored in red and blue, respectively, with the Saccharomyces-specific Mph2/3 clade in purple. Circles denote branches with >90% bootstrap support.

FIGS. 14A-14B show the similarity of chimeric maltotriose transporters. FIG. 14A: Structural models for Mty1 (left, gray and pink) and MalT434 (right, teal and orange) are shown from side-on (top row) and top-down (bottom row) views. Intracellular N- and C-terminal domains are hidden for clarity. On each structure the contrasting colors denote regions from different parental proteins (i.e., chimeric breakpoints). The two models are superimposable with RMSD=0.898 Å. FIG. 14B: Detail of amino acid similarity between MalT434 and Mty1. The structural model of MalT434 is shown, with a focus on the C-terminal helical bundle as viewed from the transport channel. N-terminal helices are hidden for clarity. Side chains are drawn for residues in MalT434 that were determined to have an effect on maltotriose transport in molecular experiments, colored according to their parental protein identity (MalT4, teal; MalT3, orange). Side chains are drawn in pink for the corresponding residues in the aligned structural model of Mty1, which is hidden for clarity; the amino acid identity for Mty1 is in parentheses.

FIGS. 15A-15E show a molecular docking analysis of MalT434. FIG. 15A: A side view of the structural model of MalT434 is shown with side chains drawn at residues 379, 505, 508, and 512. Docked maltotriose is drawn as sticks. FIG. 15B: Details of proximities and potential interactions between key residues and maltotriose. The structural model with docked ligand is shown as in (a), with obscuring helices hidden. Dotted lines show distances in angstroms between maltotriose and key residues with a functional impact on maltotriose transport that could engage in a hydrogen-bonding network. FIG. 15C: Residues Q379 and N505 could inhibit sugar transport by occlusion. Maltotriose docked to the structural model of MalT434 is shown as in (b) with a mesh surface drawn for the sugar. Side chains are drawn for residues Q379 and N505, both of which could inhibit the accommodation of the large maltotriose molecule. FIGS. 15D-15E: Docked maltotriose in the space-filling model of MalT434. The surface is colored by MalT3/MalT4 identity, except for at residues 379, 505, 508, and 512, which are colored by element. In FIG. 15D, the residues at these positions have not been reverted. In FIG. 15E, the residues at these positions have been reverted to their MalT3 (379) or MalT4 (505, 508, 512) identity, which substantially reduces the volume of the substrate pocket and introduces steric clashes with the sugar.

FIGS. 16A-16C demonstrate that expanded sets of Agt proteins are generally representative of genome-wide sugar porter complement. FIG. 16A: Time-calibrated Saccharomycotina species phylogeny, as in FIG. 7, with branches colored by order. The bar chart shows the total number of sugar porters in each genome. FIG. 16B: Scatterplot of all sugar porters versus Agt proteins encoded in each genome. Each species is a point, colored by taxonomic order (key in FIG. 16C). Inset text gives Kendall's T (p<2.2×10⁻¹⁶). FIG. 16C: Scatterplots as in (B), split by taxonomic order. Inset text gives within-order Kendall's T (***p<1.3×10⁻⁵). Correlation coefficients were not calculated for orders with too few species in our dataset. Lines and shaded regions in (B) and (C) show simple linear regressions and 95% confidence intervals of untransformed data.

FIGS. 17A-17B show an improvement in annotation quality for 332 Saccharomycotina genomes. FIG. 17A: Scatterplot of annotation completeness (% of Benchmark Universal Single-Copy Orthologs/BUSCO present in single, complete copy in annotation) for the 332 Saccharomycotina genomes used in this study (Shen et al. 2018). Completeness for previous annotations is on the x-axis, and completeness for new annotations from the current work are plotted against the y-axis. The dotted gray line shows the 1:1 null expectation of no improvement. FIG. 17B: Difference in the percentage of complete and missing BUSCO for annotations from the current study compared to previous annotations. Boxplots show median and interquartile ranges; individual genomes are plotted as points. Points in both panels are colored by taxonomic order.

### DETAILED DESCRIPTION

The present invention provides engineered Saccharomyces eubayanus MalT4 proteins that have maltotriose transport activity. Also provided are polypeptides, constructs, and vectors encoding the engineered MalT4 proteins, as well as S. eubayanus yeast cells that express the engineered MalT4 proteins and methods of using these cells to make a fermentation product, such as a lager.

The inventors have engineered several novel sugar transporter proteins that confer maltotriose utilization to the wild, cold-tolerant yeast Saccharomyces eubayanus. These novel maltotriose transporters were generated by introducing specific combinations of mutations into the wild-type S. eubayanus maltose transporter protein MalT4 (SEQ ID NO: 1), which naturally has no maltotriose transport activity.

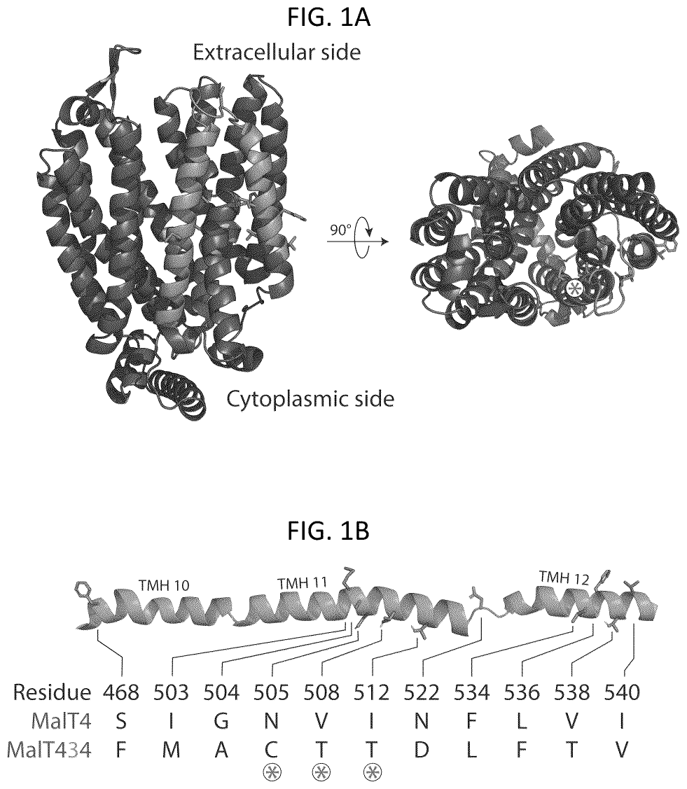

In previous work, the inventors identified a chimeric S. eubayanus sugar transporter protein, referred to as MalT434, that has maltotriose transport activity. MALT434 was generated in an adaptive evolution experiment and is the result of a recombination event between the S. eubayanus transporter genes MALT4 and MALT3 (see U.S. Pat. No. 11,028,402 and Baker and Hittinger 2019, which are each incorporated by reference in their entireties). The MalT434 protein contains 11 non-synonymous mutations relative to wild-type MalT4 (FIG. 1B).

In the Examples of the present application, the inventors dissect the molecular genetic basis of the novel maltotriose transport function of MalT434. In the process, they identify several subsets of the mutations found in MalT434 that confer maltotriose transport activity to MalT4, including (1) two minimal subsets that comprise only three mutations (N505C/V508T/I512T (SEQ ID NO: 13) and I503M/N505C/I512T (SEQ ID NO: 12)), and (2) a subset of six mutations that provide increased maltotriose transport activity relative to that of MalT434 (S468F/I503M/G504A/N505C/V508T/I512T (SEQ ID NO: 3)).

Importantly, many of the tested combinations of MalT434 mutations did not confer maltotriose transport activity to S. eubayanus. The inventors determined that the basis of maltotriose transport in MalT434 is remarkably complex, and that its novel function is shaped by a combination of additive and non-additive interactions between as many as seven regions in the transporter's backbone and six substitutions across two transmembrane helices. Thus, the combinations of MalT434 mutations that retain maltotriose transport activity could not have been predicted in the absence of the data disclosed herein.

### Engineered MalT4 Proteins:

In a first aspect, the present invention provides engineered MalT4 proteins that have maltotriose transport activity. The engineered MalT4 proteins comprise the amino acid sequence of SEQ ID NO: 1 with amino acid substitutions in 3-7 positions selected from residues 468, 503, 504, 505, 508, 512, and 379 of SEQ ID NO: 1, wherein (a) the substitution at residue 468 is S468F; (b) the substitution at residue 503 is I503M; (c) the substitution at residue 504 is G504A; (d) the substitution at residue 505 is selected from N505C, N505S, and N505G; (c) the substitution at residue 508 is V508T; (f) the substitution at residue 512 is selected from I512T and I512V; and/or (g) the substitution at residue 379 is S379C.

The proteins described herein are “engineered,” meaning that they have been altered by the hand of man. Specifically, the engineered MalT4 proteins of the present invention have been altered to comprise 3-7 amino acid substitutions relative to the sequence of the wild-type MalT4 protein, i.e., SEQ ID NO: 1. The term “wild-type” is used herein to describe the non-mutated version of a polypeptide that is most typically found in nature. The engineered MalT4 proteins may comprise 3, 4, 5, 6, or 7 amino acid substitutions relative to the wild-type MalT4 protein.

As used herein, an “amino acid substitution” refers to a change in an amino acid sequence in which one amino acid is replaced with a different amino acid. An amino acid substitution may be a conversative replacement (i.e., a replacement with an amino acid that has similar properties) or a radical replacement (i.e., a replacement with an amino acid that has different properties).

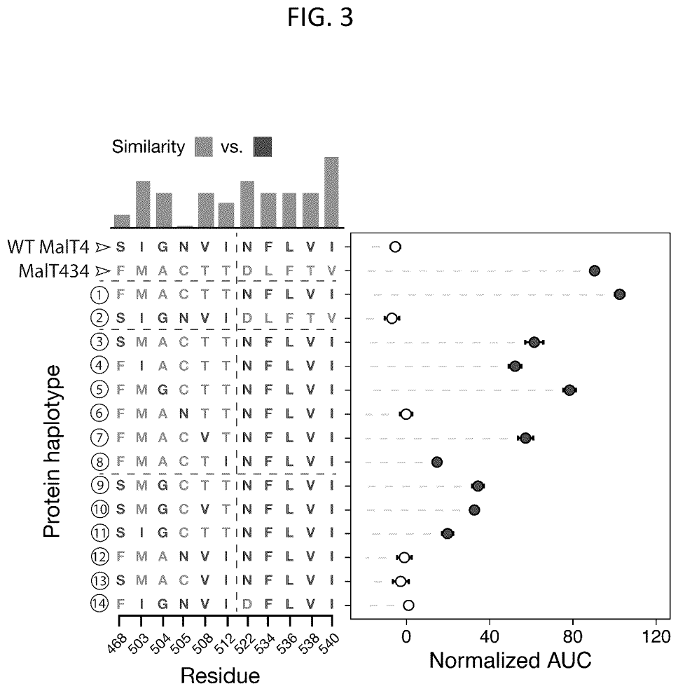

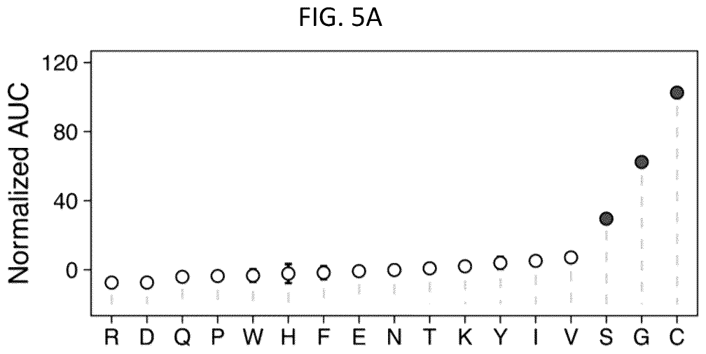

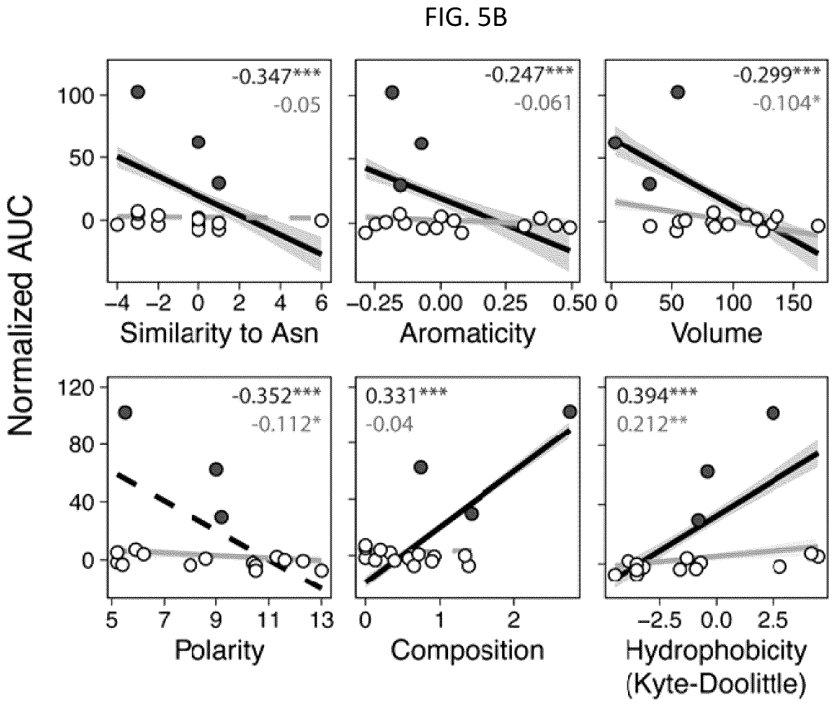

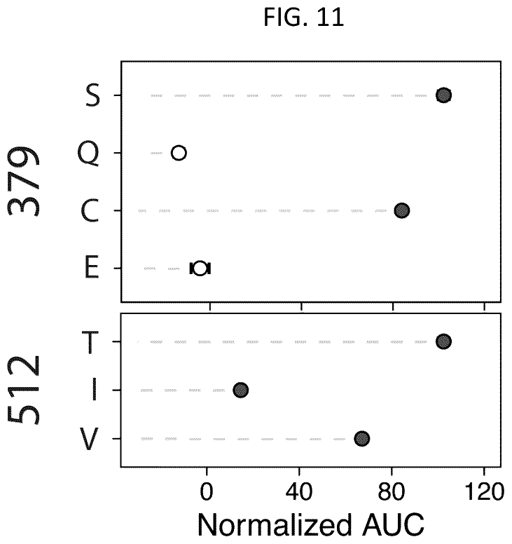

The term “maltotriose transport activity” refers to the ability of a protein to transport maltotriose across a cell membrane. In the Examples, the inventors demonstrate that engineering MalT4 to have amino acid substitutions at 3-7 positions selected from residues 468, 503, 504, 505, 508, 512, and 379 of SEQ ID NO: 1 confers maltotriose transport activity. Specifically, in FIG. 3, the inventors demonstrate that the engineered MalT4 proteins of SEQ ID NO: 3 (S468F/I503M/G504A/N505C/V508T/I512T), SEQ ID NO: 5 (I503M/G504A/N505C/V508T/I512T), SEQ ID NO: 6 (S468F/G504A/N505C/V508T/I512T), SEQ ID NO: 7 (S468F/I503M/N505C/V508T/I512T), SEQ ID NO: 9 (S468F/I503M/G504A/N505C/I512T), SEQ ID NO: 10 (S468F/I503M/G504A/N505C/V508T), SEQ ID NO: 11 (I503M/N505C/V508T/I512T), SEQ ID NO: 12 (I503M/N505C/I512T), and SEQ ID NO: 13 (N505C/V508T/I512T) have maltotriose transport activity. In FIG. 5, they demonstrate that the MalT4 mutants of SEQ ID NO: 17 (S468F/I503M/G504A/N505S/V508T/I512T) and SEQ ID NO: 18 (S468F/I503M/G504A/N505G/V508T/I512T) have maltotriose transport activity. And in FIG. 11, they demonstrate that the MalT4 mutants of SEQ ID NO: 19 (S468F/I503M/G504A/N505C/V508T/I512V) and SEQ ID NO: 20 (S379C/S468F/I503M/G504A/N505C/V508T/I512T) have maltotriose transport activity. Thus, in some embodiments, the engineered MalT4 protein has an amino acid sequence selected from SEQ ID NOs: 3, 5-7, 9-13, and 17-20. The SEQ ID NOs for the various MalT4 mutants that were tested in the Examples are presented in Table 1, below.

TABLE I
Sequences of the new MalT4 mutants tested herein

| SEQ | | |
|---|---|---|
| ID NO: | Mutations | Figure |
| 3 | S468F/I503M/G504A/N505C/V508T/I512T | FIG. 3 |
| 4 | N522D/F534L/L536F/V538T/I540V | FIG. 3 |
| 5 | I503M/G504A/N505C/V508T/I512T | FIG. 3 |
| 6 | S468F/G504A/N505C/V508T/I512T | FIG. 3 |
| 7 | S468F/I503M/N505C/V508T/I512T | FIG. 3 |
| 8 | S468F/I503M/G504A/V508T/I512T | FIG. 3 |
| 9 | S468F/I503M/G504A/N505C/I512T | FIG. 3 |
| 10 | S468F/I503M/G504A/N505C/V508T | FIG. 3 |
| 11 | I503M/N505C/V508T/I512T | FIG. 3 |
| 12 | I503M/N505C/I512T | FIG. 3 |
| 13 | N505C/V508T/I512T | FIG. 3 |
| 14 | S468F/I503M/G504A | FIG. 3 |
| 15 | I503M/G504A/N505C | FIG. 3 |
| 16 | S468F/N522D | FIG. 3 |
| 17 | S468F/I503M/G504A/N505S/V508T/I512T | FIG. 5 |
| 18 | S468F/I503M/G504A/N505G/V508T/I512T | FIG. 5 |
| 19 | S468F/I503M/G504A/N505C/V508T/I512V | FIG. 5 |
| 20 | S379C/S468F/I503M/G504A/N505C/V508T/I512T | FIG. 5 |

The inventors identified one engineered MalT4 protein, i.e., that of SEQ ID NO: 3 ((S468F/I503M/G504A/N505C/V508T/I512T), that has increased maltotriose transport activity as compared to the previously disclosed MalT4 variant referred to as MalT434 (SEQ ID NO: 2) (see FIG. 3; compare rows two and three). Thus, in some embodiments, the engineered MalT4 protein has increased maltotriose transport activity as compared to MalT434. In some embodiments, the maltotriose transport activity of the engineered MalT4 protein is increased by at least 10%, 20%, 30%, 40%, 50%, 55%, 60%, 65%, 70%, 75%, 80%, 85%, 90%, 95%, 100%, 150%, 200%, 300%, 500%, or 1000% as compared to the maltotriose transport activity of MalT434. The maltotriose transport activity of a protein can be measured by introducing the protein into a yeast strain that does not naturally express a maltotriose transporter protein (e.g., S. eubayanus) and measuring its ability to grow on a medium comprising maltotriose as the sole carbon source. Alternatively, the maltotriose transport activity of a protein can be measured by introducing the protein into a yeast, culturing the yeast in a medium comprising maltotriose, and measuring (a) the depletion of maltotriose from the medium (e.g., using separation by high-performance liquid chromatography (HPLC) coupled with spectroscopic quantification or radiometric measurement of radiolabeled sugars), or (b) the depletion of co-transported protons from the medium (e.g., using a pH probe).

In the Examples, the inventors demonstrate that engineered MalT4 proteins that have an N505C, N505S, or N505G substitution at residue 505 have maltotriose transport activity (FIG. 5). They also demonstrate that engineered MalT4 proteins that have an I512T or I512V substitution at residue 512 or an S379C substitution as residue 379 have maltotriose transport activity (FIG. 11). Thus, in some embodiments, the engineered MalT4 protein is a variant of SEQ ID NO: 3 having (a) a different substitution at residue 505, (b) a different substitution at residue 512, and/or (c) an additional substitution at residue 379. Specifically, these variants comprise (a) the amino acid substitutions S468F, I503M, G504A, and V508T; (b) a substitution at residue 505 selected from N505C, N505S, and N505G; and (c) a substitution at residue 512 selected from I512T and I512V; and they optionally further comprise the amino acid substitution S379C.

The terms “protein” and “polypeptide” refer to a series of amino acid residues connected by peptide bonds between the alpha-amino and carboxy groups of adjacent residues, forming a polymer of amino acids. In the present application, the term “protein” and “polypeptide” are used interchangeably. Proteins/polypeptides may include modified amino acids. Suitable modifications include, but are not limited to, acylation, acetylation, formylation, lipoylation, myristoylation, palmitoylation, alkylation, isoprenylation, prenylation, amidation at C-terminus, glycosylation, glycation, polysialylation, glypiation, and phosphorylation. Proteins/polypeptides may also include amino acid analogs.

The engineered proteins of the present invention may further comprise additional polypeptides, such as protein tags. As used herein, a “protein tag” is a polypeptide that is added to recombinant protein to provide a particular function. Examples of suitable protein tags include, without limitation, affinity tags for protein purification (e.g., chitin binding protein (CBP), maltose binding protein (MBP), Strep, glutathione-S-transferase (GST), and poly(His) tags), solubilization tags (e.g., thioredoxin (TRX), poly (NANP), MBP, GST), epitope tags for antibody-based detection (e.g., ALFA-tag, V5-tag, Myc-tag, HA-tag, Spot-tag, T7-tag and NE-tag), and fluorescent tags (e.g., green fluorescent protein (GFP), red fluorescent protein (RFP)), and enzymatic tags (e.g., horseradish peroxidase, alkaline phosphatase, beta-galactosidase, glucose-6-phosphatase, acetylcholinesterase) for visual detection.

### Polynucleotides:

In a second aspect, the present invention provides polynucleotides encoding an engineered MalT4 proteins disclosed herein.

The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages.

Those of skill in the art understand that, due to the degeneracy of the genetic code, a variety of polynucleotides can encode the same polypeptide. Any polynucleotide sequence that encodes the desired engineered MalT4 protein may be utilized. In some embodiments, the polynucleotide (or the portion of the polynucleotide encoding the engineered MalT4 protein) is codon-optimized for expression in a particular cell (e.g., a plant cell, bacterial cell, or fungal cell). “Codon optimization” is a process used to increase the expression of a polynucleotide in a particular host cell by altering the sequence of the polynucleotide to accommodate the codon bias of the host cell. For example, for expression in an S. eubayanus yeast cell, the polynucleotide can include the codons most frequently found in the genome of that yeast cell for efficient expression of the engineered MalT4 protein in that cell. Computer programs for generating codon-optimized sequences for use in a particular host cell are known in the art.

### Constructs:

In a third aspect, the present invention provides constructs comprising a promoter operably linked to a polynucleotide disclosed herein.

The term “construct” refers to a recombinant polynucleotide, i.e., a polynucleotide that was formed by combining at least two polynucleotide components from different sources. For example, a construct may comprise the coding region of one gene operably linked to a promoter that is (1) associated with another gene found within the same genome, (2) from the genome of a different organism, or (3) synthetic. Constructs can be generated using conventional recombinant DNA methods.

As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide. The promoter used in the constructs described herein may be either a native MalT4 promoter or a heterologous promoter (i.e., a promoter that is not naturally associated with the MalT4 gene). The promoter may be a naturally occurring promote or it may be synthetic. The promoter may be a yeast promoter (i.e., a promoter found in yeast), or it may be from another organism. Suitable promoters include, but are not limited to, constitutive, inducible, temporally regulated, and developmentally regulated promoters.

### Vectors:

In a fourth aspect, the present invention provides vectors comprising a polynucleotide or construct disclosed herein. The term “vector” refers to a DNA molecule that is used to carry a particular DNA segment (i.e., a DNA segment included in the vector) into a host cell. Some vectors are capable of autonomous replication in a host cell (e.g., bacterial vectors and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene (e.g., a gene encoding a fluorescent protein) or a selectable marker gene (e.g., a gene that confers antibiotic resistance).

In some embodiments, the vector is a plasmid or a yeast artificial chromosome. A “plasmid” is a small circular DNA molecule that can replicate independently from chromosomal DNA. In nature, plasmids are commonly found in bacteria, and artificial plasmids are widely used as vectors in molecular cloning. A yeast artificial chromosome (YAC) is an engineered DNA molecule used for cloning DNA sequences in yeast cells. A YAC generally includes yeast telomeric sequences, a yeast centromere, a yeast autonomously replicating sequence, and it may also include selectable markers, detectable markers, recombinant genes, and regulatory elements.

### Yeast:

In a fifth aspect, the present invention provides Saccharomyces eubayanus yeast cells comprising an engineered MalT4 protein, polynucleotide, construct, or vector described herein.

Wild-type S. eubayanus lack the ability to transport maltotriose across their plasma membrane and, as a result, cannot use maltotriose as a carbon source. In the Examples, the inventors demonstrate that expressing the engineered MalT4 proteins described herein in S. eubayanus confers the ability to transport and utilize maltotriose. Thus, in preferred embodiments, the yeast cell has maltotriose transport activity.

In some embodiments, the yeast cell has increased maltotriose transport activity as compared to a control yeast cell that expresses MalT434 (SEQ ID NO: 2). The maltotriose transport activity of the yeast cell may be increased by at least 30%, 40%, 50%, 55%, 60%, 65%, 70%, 75%, 80%, 85%, 90%, 95%, 100%, 150%, 200%, 300%, 500% or 1000% as compared to the control yeast cell. As used herein, a “control yeast cell” is a comparable yeast cell (e.g., of the same strain) that was grown under substantially similar conditions but that expresses the previously disclosed MalT4 variant MalT434 rather than a MalT4 variant described herein.

In the Examples, the inventors demonstrate that expressing the engineered MalT4 proteins described herein in the yeast strain yHJC207, a haploid derivative of yHKS210, confers maltotriose transport activity to this yeast. Thus, in some embodiments, the yeast cell is strain yHJC207 or yHKS210.

In some embodiments, the yeast cells are modified to express an engineered MalT4 protein disclosed herein via introduction of a polynucleotide, construct, or vector encoding said protein. Methods of introducing polynucleotides into a cell are known in the art and may include, without limitation, microinjection, transformation, and transfection methods. In some embodiments, the polynucleotide in integrated into the yeast genome. In other embodiments, the polynucleotide remains extrachromosomal.

In some embodiments, the yeast cells are modified to express an engineered MalT4 protein via genome editing. Genome editing may be used, for example, to modify the native MALT4 gene to include a set of 3-7 amino acid substitutions disclosed herein. Alternatively, genome editing may be used to replace the native MALT4 gene with a polynucleotide encoding a MalT4 variant disclosed herein, or to insert a MALT4 variant disclosed herein elsewhere in the yeast genome. Genome editing methods are known in the art and include, but are not limited to, CRISPR/Cas9-based genome editing.

Notably, once a polynucleotide encoding an engineered MalT4 protein has been engineered or stably introduced into the genome of a yeast strain, the modified yeast strain can be used to introduce the polynucleotide into other genetic backgrounds of S. eubayanus via traditional breeding methods.

### Methods:

In a sixth aspect, the present invention provides methods for making a fermentation product. The methods comprise culturing a yeast cell described herein with a fermentable substrate.

Yeast fermentation is a process in which yeast convert carbohydrates into alcohol and carbon dioxide. Fermentation is accomplished by culturing yeast cells (i.e., growing them in an artificial environment) in a culture medium that comprises a fermentable substrate in the absence of oxygen. As used herein, the term “fermentable substrate” refers to a carbohydrate (i.e., a sugar or starch) that can be converted into alcohol and carbon dioxide by yeast. Suitable fermentable substrates include wort, mash, dough, and malt extract. In preferred embodiments, the fermentable substrate comprises the sugar maltotriose.

The methods of the present invention may be used to produce any yeast fermentation product. Examples of suitable yeast fermentation products include, without limitation, beverages, foods (e.g., bread), biochemicals, and biofuels. Yeast fermented beverages include both alcoholic beverages, such as beer and wine, and non-alcoholic and low-alcohol beverages. In certain embodiments, the fermentation product is a lager.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Example 1

In the following Example, the inventors dissect the molecular genetic basis of novel function in the chimeric Saccharomyces eubayanus maltotriose transporter MalT434. In the process, they identify several mutant forms of the S. eubayanus maltose transporter MalT4 that have maltotriose transport activity.

Functional innovation at the protein level is a key source of evolutionary novelties. The constraints on functional innovations are likely to be highly specific in different proteins, which are shaped by their unique histories and the extent of global epistasis that arises from their structures and biochemistries. These contextual nuances in the sequence-function relationship have implications both for a basic understanding of the evolutionary process and for engineering proteins with desirable properties. Here, we have investigated the molecular basis of novel function in a model member of an ancient, conserved, and biotechnologically relevant protein family. These Major Facilitator Superfamily sugar porters are a functionally diverse group of proteins that are thought to be highly plastic and evolvable. By dissecting a recent evolutionary innovation in an α-glucoside transporter from the yeast Saccharomyces eubayanus, we show that the ability to transport a novel substrate requires high-order interactions between many protein regions and numerous specific residues proximal to the transport channel. To reconcile the functional diversity of this family with the constrained evolution of this model protein, we generated new, state-of-the-art genome annotations for 332 Saccharomycotina yeast species spanning approximately 400 million years of evolution. By integrating phylogenetic and phenotypic analyses across these species, we show that the model yeast α-glucoside transporters likely evolved from a multifunctional ancestor and became subfunctionalized. The accumulation of additive and epistatic substitutions likely entrenched this subfunction, which made the simultaneous acquisition of multiple interacting substitutions the only reasonably accessible path to novelty.

### Introduction

Many key evolutionary innovations arise from changes to protein sequences that alter their function (Cheng 1998; Zhang et al. 2002; Clark et al. 2003; Dorus et al. 2004; Lunzer et al. 2005; Nielsen et al. 2005; Hoekstra et al. 2006; Christin et al. 2007; Yokoyama et al. 2008; Voordeckers et al. 2012; Projecto-Garcia et al. 2013; Kaltenbach et al. 2018; Jabłońska and Tawfik 2022). Occasionally, these changes stem from dramatic mutational events, including the creation of highly novel coding sequences by gene conversion or ectopic recombination resulting in chimeric proteins (Long and Langley 1993; Nurminsky et al. 1998; Wang et al. 2000; Long et al. 2003; Patthy 2003; Zhang et al. 2004; Ciccarelli et al. 2005; Arguello et al. 2006; Rogers et al. 2010; Rogers and Hartl 2012; Leffler et al. 2017; Meheust et al. 2018; Baker and Hittinger 2019; Brouwers, Gorter de Vries, et al. 2019; Smithers et al. 2019; Baker et al. 2022). While gene conversion can theoretically accelerate the rate of evolution (or even enable adaptation altogether) by bypassing deleterious intermediates, this effect is primarily attributable to the presence of a rugged fitness landscape (Kauffman and Levin 1987; HANSEN et al. 2000; Cui et al. 2002; Bittihn and Tsimring 2017). Such rugged landscapes are manifestations of epistasis in the genotypic combinations underlying the phenotypic map and are prevalent in some empirical systems (Wright 1931; Wright 1932; Maynard Smith 1970; Townsend et al. 2003; Weinreich et al. 2005; Weinreich et al. 2006; Gong et al. 2013; Weinreich et al. 2013; De Visser and Krug 2014; Sarkisyan et al. 2016; Starr and Thornton 2016; Wu et al. 2016; Pokusaeva et al. 2019; Yi and Dean 2019; Nishikawa et al. 2021; Park et al. 2022; Meger et al. 2024; Metzger et al. 2024). For other proteins, the fitness landscape may be much smoother, meaning that stepwise mutations with additive effects can underlie functional evolution (Lunzer et al. 2005; Bridgham et al. 2006; Weinreich et al. 2006; Poclwijk et al. 2007; Campbell et al. 2016; Kaltenbach et al. 2018; Srikant et al. 2020). In cases where novel protein function is linked to gene conversion events between homologs, these observations therefore raise a fundamental question: are such dramatic mutational events required to evolve new function, or are they probabilistic shortcuts in the evolutionary process whose prevalence is a predictable function of their combined effect size and relative mutation rate? Answering this question has significant implications for understanding and predicting the most likely evolutionary trajectories, as well as for designing and engineering novel proteins with desirable functions.

Recently, several remarkably parallel cases of functional innovation have been linked directly or speculatively to gene conversion events in an ecologically and biotechnologically relevant protein family: maltose transporters in Saccharomyces yeasts (Baker and Hittinger 2019; Brouwers, Gorter de Vries, et al. 2019; Hatanaka et al. 2022). This protein family consists of transporters similar to the Saccharomyces cerevisiae Mal31 protein, which has high specificity and high affinity for the disaccharide maltose, which contains two glucose moieties (Cheng and Michels 1991; Stambuk and Araujo 2001; Salema-Oom et al. 2005; Alves et al. 2008; Brown et al. 2010). Mal31-like proteins are encoded in nearly all genomes of Saccharomyces and some closely related species, and they are frequently encoded by multiple paralogs within each genome.

Maltose uptake is also mediated by a second family of proteins, which are related to S. cerevisiae Agt1. In contrast to the Mal31-like proteins, Agt1 is a generalist α-glucoside transporter with a broad substrate range, but it has generally lower affinity for those substrates (Han et al. 1995; Stambuk et al. 1999; Stambuk et al. 2000; Alves et al. 2008; Trichez et al. 2019). Notably, Agt1 can transport the glucose trisaccharide maltotriose, a molecule that is biochemically similar to maltose but contains a third glucose moiety. Although sometimes referred to as Mal11, Agt1 is a functionally distinct protein with ˜57% amino acid sequence identity to the Mal31-like proteins. In contrast to the Mal31-like proteins, Agt1-like proteins are rarer, both in presence and in paralog number, in the genomes of Saccharomyces yeasts and close relatives (Duval et al. 2010; Horák 2013).

The α-glucoside transporters (Agts) of Saccharomyces include the Agt1-like (“generalist”) and Mal31-like (“high-specificity”) proteins, as well as Mph2/3-like proteins (Day et al. 2002), which also have high specificity, albeit for the α-glucoside turanose (Brown et al. 2010). These Agts have been extensively studied due to their important role in the production of beer. Maltose and maltotriose are the two most abundant sugars in brewer's wort (Meussdorfer and Zarnkow 2009), and their transport into the cell is the rate-limiting step in their fermentation (Zastrow et al. 2001; Horák 2013). The rarity of maltotriose transporters, such as Agt1, which almost always manifests as an inability to ferment this carbon source, therefore presents a barrier to the use of many non-domesticated yeasts in brewing applications.

This barrier is exemplified in Saccharomyces eubayanus, the wild, cold-tolerant parent of industrial lager-brewing hybrids (Libkind et al. 2011), whose development for commercial brewing is of great interest (Gibson et al. 2017; Hittinger et al. 2018; Cubillos et al. 2019). As almost all strains of S. eubayanus lack generalist Agts capable of transporting maltotriose (Brickwedde et al. 2018; Brouwers, Brickwedde, et al. 2019; Bergin et al. 2022), multiple attempts have been made to evolve maltotriose transporters de novo in S. eubayanus strains, using both mutagenesis (Brouwers, Gorter de Vries, et al. 2019) and adaptive laboratory evolution (Baker and Hittinger 2019). These experiments, performed independently in different backgrounds of S. eubayanus, yielded results that were as remarkable in their similarity as they were unexpected. In both cases, ectopic gene conversion between paralogous high-specificity (Mal31-like) maltose transporters without any native maltotriose transport capacity (Brickwedde et al. 2018; Baker and Hittinger 2019) resulted in chimeric proteins capable of transporting maltotriose.

Lending weight to the notion that recombination may be a common mechanism by which transporters in the high-specificity Agt family evolve new function, two newly discovered S. cerevisiae transporters (Hatanaka et al. 2022), as well as the Mty1 protein (Dietvorst et al. 2005; Salema-Oom et al. 2005), may possess signatures of more ancient gene conversion events (Brouwers, Gorter de Vries, et al. 2019). All these proteins transport maltotriose, but they cluster with Mal31-like proteins in phylogenetic analyses (Baker and Hittinger 2019; Hatanaka et al. 2022). Nonetheless, it remains unclear whether these dramatic mutational events are required for the evolution of novel function in this family or whether they are simply enriched due to the dynamic nature of the subtelomeric regions in which these genes reside (Mefford and Trask 2002; Fairhead and Dujon 2006; Gordon et al. 2009; Brown et al. 2010; Yue et al. 2017; Peter et al. 2018; Liu et al. 2019; O'Donnell et al. 2023).

The yeast α-glucoside transporters are H⁺ symporters belonging to the sugar porter family (TCDB: 2.A.1.1) of the Major Facilitator Superfamily (MFS), a vast, ubiquitous, and ancient group of transmembrane proteins present in all domains of life (Marger and Saier 1993; Pao et al. 1998; Saier 2000; Wang et al. 2020; Saier et al. 2021). Across great evolutionary distances, sugar porters share the highly characteristic MFS fold consisting of twelve transmembrane helices (TMHs) surrounding a hydrophilic central cavity that constitutes the transport channel (Abramson et al. 2003; Guan and Kaback 2006; Sun et al. 2012; Deng et al. 2014; Quistgaard et al. 2016; Bosshart and Fotiadis 2019; Kaback and Guan 2019; Paulsen et al. 2019; Drew et al. 2021). These TMHs are organized into two pseudosymmetrical six-helix bundles (N- and C-terminal), which are separated by a long intracellular linker (ICH domain). The transport channel is surrounded by four helices from each bundle, and TMHs stack tightly against their intra-bundle partners, with additional contacts between the N- and C-terminal domains at the inter-bundle interface. In S. cerevisiae Agt1, the sugar substrate and/or proton are thought to be bound primarily by charged residues projecting into this central cavity, which are conserved across fungal Agts (Henderson and Poolman 2017; Trichez et al. 2019). More generally, substrate affinity and specificity in MFS sugar transporters are mediated by extensive hydrogen bonding and occasionally by hydrophobic interactions between the sugar and the protein, as well as steric constraints that limit substrate accommodation; moreover, there is a growing appreciation for the fine-scale and occasionally cryptic contributions to affinity by residues within Van der Waals distance of the substrate (Kasahara et al. 1997; Kasahara and Kasahara 1998; Kasahara and Kasahara 2000; Guan and Kaback 2006; Kasahara et al. 2006; Guan et al. 2007; Kasahara et al. 2007; Kasahara et al. 2009; Kasahara and Kasahara 2010; Kasahara et al. 2011; Sun et al. 2012; Deng et al. 2014; Farwick et al. 2014; Deng et al. 2015; Bosshart and Fotiadis 2019; Kaback and Guan 2019; Drew et al. 2021; Guan and Hariharan 2021).

Nonetheless, the extensive and exquisite biochemical study of MFS sugar transporters has almost exclusively focused on the determinants of native substrate binding and affinity in extant proteins, while questions about how such proteins could evolve the capacity to transport a novel substrate de novo have been largely unaddressed. Understanding evolution-informed design principles in this protein family could enable the engineering of desirable properties in tractable proteins, with significant implications for industrial processes, including the fermentation of cellulosic and hemicellulosic biomass into next-generation biofuels and bioproducts (Ha et al. 2013; Farwick et al. 2014; Young et al. 2014; Turner et al. 2016; Hara et al. 2017; Oh et al. 2017; Casa-Villegas et al. 2018; Kim et al. 2018; Nijland et al. 2018; Nijland and Driessen 2020; Oh and Jin 2020; de Ruijter et al. 2020).

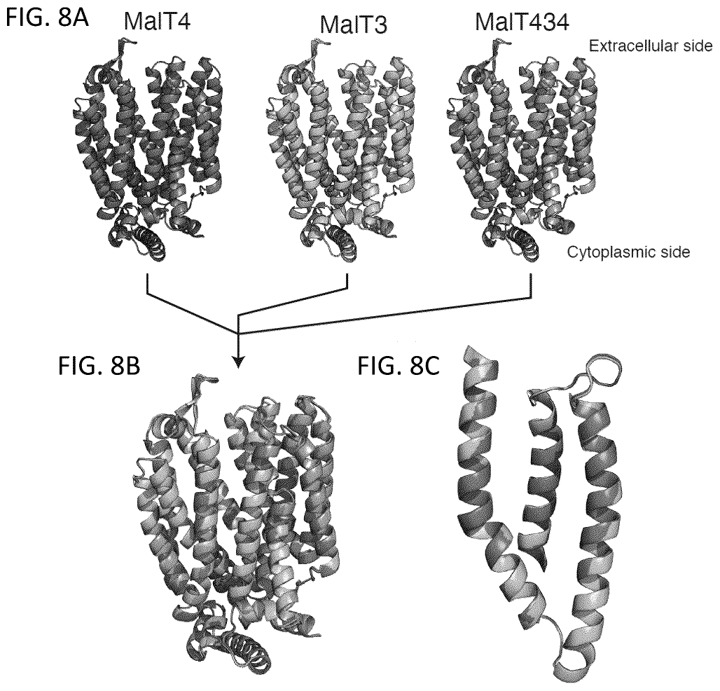

To this end, we aimed to dissect the molecular genetic basis of novel function in the chimeric S. eubayanus maltotriose transporter MalT434. MALT434 arose from an ectopic gene conversion event between genes encoding two paralogous maltose transporters, MalT3 and MalT4, which resulted in the replacement of approximately 230 base pairs of the MALT4 gene with the homologous portion of MALT3 (Baker and Hittinger 2019). Both MalT3 and MalT4 are members of the high-specificity maltose transporter family and incapable of transporting maltotriose (Brickwedde et al. 2018; Baker and Hittinger 2019), suggesting that intramolecular epistasis between their protein regions underlies the emergent maltotriose transport by MalT434. The translocated region of MALT3 encodes TMH 11 and portions of TMHs 10 and 12 (FIG. 1A), and it introduced 11 nonsynonymous mutations to the protein-coding sequence of MALT4 (FIG. 1B). All three proteins are predicted to have virtually identical structures across their entire folds (pairwise RMSD-0.955 Å) and TMHs 10⁻¹² (0.909 Å, FIG. 8), suggesting that novel substrate transport might stem from a specific combination of substrate-interacting residues from distal protein regions in MalT434, rather than a global change to protein structure. In the simplest model, as few as a single interacting residue from each protein region could underlie the emergence of novel function, which would make the evolution of new function in this family predictable and tunable; in the most complex model, all 120 amino acid differences between the two parental transporters could contribute, which would render the evolution of new function incredibly difficult.

Here, we show that the basis of maltotriose transport is remarkably complex in this model neofunctionalized transporter. Novel function is shaped by a combination of additive and non-additive interactions between as many as seven regions in the MalT4 backbone and six substitutions across TMHs 10 and 11. At one critical site, very few amino acids can support novel function, which further limits the evolutionary paths available to the wild-type protein; at other sites, these requirements are less stringent. We propose that, overall, novel substrate transport is enabled by widening the transport channel while simultaneously creating a favorable electrostatic environment for the bulkier trisaccharide molecule. Finally, we reconstruct the evolutionary history of the high-specificity and generalist yeast Agts and their relationships to other sugar porters; unexpectedly, we show that the specialist maltose transporters are likely derived and subfunctionalized from a generalist ancestor. This specialization likely involved a gradual refinement of the transport channel to specifically accommodate maltose with higher affinity, which makes the reacquisition of ancestral generalist function difficult to achieve. While our results indicate that rational engineering for novel substrate transport in this protein family is likely to be difficult, they also highlight the abundance and diversity of transporters in biotechnologically relevant yeast species, which could be readily mined for desirable functions that have been exquisitely refined over billions of years of evolution, as well as perhaps recombined into new functions.

### Results:

### High-Order Intramolecular Interactions are Required to Evolve a Novel Function in Maltose Transporters

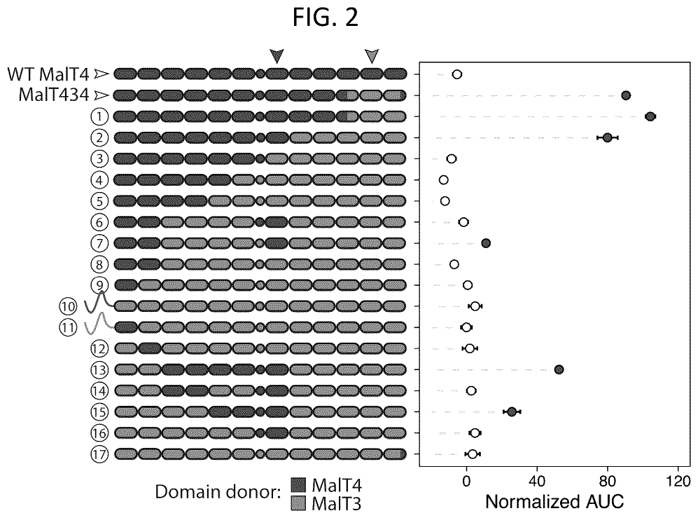

We first investigated the scope and complexity of intramolecular interactions shaping the emergence of novel function in MalT434. We defined functional protein units as the twelve transmembrane helices (TMHs), the intracellular (ICH) domain, and the partially unstructured intracellular N- and C-terminal regions, which were chosen based on these units' separable roles in substrate binding and translocation. We iteratively constructed novel chimeric genes encoding transporters from these MalT3 and MalT4 components and tested their ability to support growth on maltotriose when expressed from the native MALT4 locus (FIG. 2). Unsurprisingly, the C-terminal portion of MalT4 present in MalT434 was neither necessary (construct 1) nor sufficient (construct 17) for maltotriose transport; indeed, its replacement with the corresponding region of MalT3 improved growth on maltotriose by 15.3% (p=5.3×10⁻⁴, Mann-Whitney U test). By contrast, replacement of TMHs 8 and 9 and the N-terminal half of TMH 10 with their MalT3 counterparts (construct 2) reduced growth by 11.6% compared to MalT434 (p=0.184), while still supporting robust growth. Dissection of the region N-terminal to TMH 8 revealed that the key interaction enabling maltotriose transport occurs between one or more of TMHs 10⁻¹² of MalT3 and TMH 7 from MalT4. While necessary, this region alone was not sufficient to enable maltotriose transport in every protein context. In addition to the epistatic interaction between TMHs 7 and 10⁻¹², growth on maltotriose required the presence of TMHs 1 and 2 from MalT4 in combination with the ICH domain from MalT3 (construct 7), or alternatively, one or more of TMH 5, TMH 6, and the ICH domain from MalT4 (construct 15).

For chimeric constructs containing potentiating sequences at TMHs 5-7 and 10⁻¹², growth on maltotriose generally increased additively with the number of MalT4 regions incorporated (linear regression, p<2.2×10⁻¹⁶). Nonetheless, we found significant support (ANOVA, p<2.2×10⁻¹⁶) for pairwise epistasis between the tested protein regions, including in the sign of the effects of the ICH domain and the C-terminal region (residues 541-613). For example, the addition of TMH 3 and TMH 4 from MalT4 in conjunction with MalT4 TMH 7 only increased growth on maltotriose if TMH 5 and TMH 6 from MalT4 were also present; similarly, the addition of TMH 1, TMH 2, and the ICH domain from MalT4 in conjunction with TMH 7 did not improve growth (construct 6 vs. 16, FIG. 2) unless in the presence of TMHs 3-6 from MalT4 (construct 2 vs. 13, 52% increase, p=2.4×10⁻⁴). Along the quantitative functional spectrum of MalT3/4 chimeric proteins enabling growth on maltotriose, we therefore detected a complex combination of additive and epistatic intramolecular interactions among at least six protein regions.

### Numerous Substitutions are Required to Evolve a Novel Function in Maltose Transporters

We next dissected the contributions of the 11 substitutions in MalT434 relative to MalT4 (FIG. 1B) by introducing subsets of these to the gene encoding the native MalT4 protein (FIG. 3). We first tested the effect of a pair of suggestive substitutions, S468F and N522D, which were both unique in their location in the tertiary structure and differed notably in side-chain chemistry (protein 14, FIG. 3). Nonetheless, this pair of substitutions was insufficient for novel function in MalT4, so we coarsely tested the effect of the sets of substitutions occurring before and after the end of TMH 11. Introduction of the five substitutions between residues 522 and 540, which span an extracellular loop and the majority of TMH 12, was insufficient to confer any growth on maltotriose (protein 2, FIG. 3). By contrast, the six mutations affecting TMHs 10 and 11 were sufficient to confer growth on maltotriose (protein 1), and even improved it by 13.3% relative to MalT434 (p=5.6×10⁻⁷, Mann-Whitney U test). Within this contiguous patch of substitutions, however, the contribution of individual amino acids to novel function was remarkably complex. Reversion of the six mutations singly to their MalT4 identity revealed that each had a significant effect on maltotriose growth, ranging from a 23.5% reduction (A504G, protein 5; p=2×10⁻⁶) to its complete abrogation (C505N, protein 6; p=5.2×10⁻¹¹), with an average effect of 57.1%. We detected significant (p<2.2×10⁻¹⁶) evidence of pairwise epistasis between substitutions, regardless of whether we considered all 11 sites or only the 6 on TMHs 10 and 11. Epistatic effects were notably non-uniform among tested combinations: for example, two single reversion mutations (M5031/protein 4 and T508V/protein 7) had similar effects of 49.1% (p=3.2×10⁻⁷) and 44.1% (p=9.1×10⁻¹³) when introduced in the six-substitution background that supported robust growth on maltotriose. By contrast, when introduced in a four-substitution background with reduced ability to support growth on maltotriose (protein 9), the effect of M5031 remained large (42.6%, p=0.002), while T508V effected only a small further reduction (4.97%, protein 10; p=0.8). Overall, we found that establishing novel function in MalT4 required a combination of three amino acid substitutions only accessible through a minimum of four non-consecutive nucleotide substitutions to the wild-type gene: N505C (2 nucleotide substitutions), I512T (1 substitution), and one of I503M (1 substitution) or V508T (2 substitutions).

### Granular Mapping of Epistasis Between Distal Protein Regions

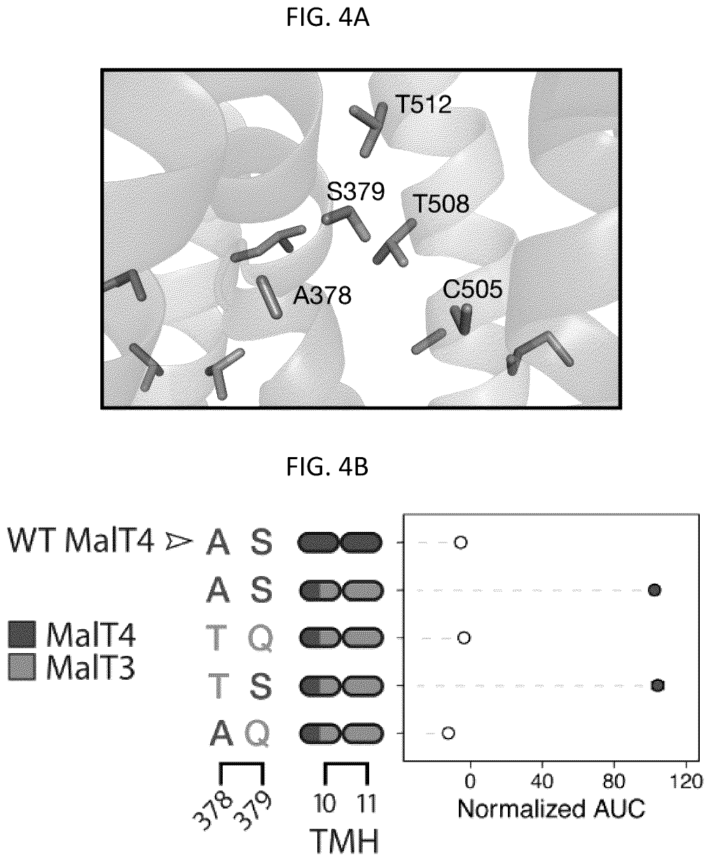

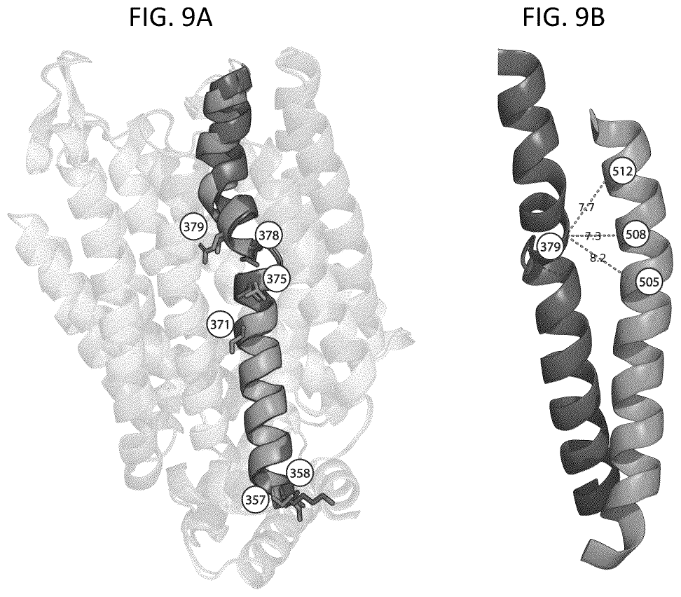

Given the size of interacting protein regions and the complexity of their contributions to novel function, we sought to identify the key difference in amino acid sequence responsible for the large epistatic effect of transmembrane helix 7. The two parental transporters differ at six sites along TMH 7 (FIG. 9A): two neighboring substitutions (K357C and V358I, expressed relative to MalT4) occur at the intracellular C-terminal end, while two (A371I, V375T) are located approximately halfway along the helix and likely to be embedded in the plasma membrane. Two (A378T, S379Q) project into or neighbor the transport channel, differ in size and/or polarity, and are in close three-dimensional proximity to mutated residues on TMH 11 in MalT434 (FIG. 4A, FIG. 9B). We reasoned that one or both of A378T and S379Q might have a large effect on the interaction between TMH 7 and the translocated region of MalT3 present in functional chimeric transporters. To test these hypotheses, we mutated each of these residues to their MalT3 identity, singly and in combination, in a gene encoding the MalT4 transporter harboring the six mutations on TMHs 10 and 11 that conferred maximal maltotriose transport (FIG. 4B). While the A378T mutation did not affect growth on maltotriose, S379Q abolished it completely. The large epistatic interaction between TMH 7 and TMH 11 can thus be attributed to a single amino acid.

### Novel Transporter Function is Constrained by Specific Biochemical Requirements and Context

The mutational event that generated MalT434, as well as our experiments dissecting it, only sampled variation between two binary states: the specific amino acid residues of the parental proteins at each homologous site. In native contexts, however, many more amino acid substitutions are accessible in mutational space through single- or multi-nucleotide mutations; for example, seven amino acid substitutions require only a single nucleotide change from an asparagine codon, which is the wild-type amino acid at the crucial 505 site. While we found complex interactions between many sites to contribute to novel function in MalT4, the evolution of maltotriose transport would be far less constrained and more accessible through sequential point mutations if biochemically similar amino acids at key sites could enable a degree of novel function because it would increase the mutational target size and pool of mutations conferring a fitness benefit (Miyazaki and Arnold 1999; Podgornaia and Laub 2015).

We thus sought to clarify the biochemical requirements for maltotriose transport in a specific potentiated context: a MalT4 transporter harboring S379, F468, M503, A504, T508, and T512. In this state, amino acid identity at position 505 is crucial with the wild-type asparagine incapable of supporting growth on maltotriose and the recombinant cysteine supporting robust growth (FIG. 3). We successfully mutated this residue to 17 of the 20 possible amino acids, measured their ability to support growth on maltotriose, and used regression analyses to estimate the effect of side chain physicochemical properties on measured function (FIG. 5). Remarkably, only three substitutions supported any degree of statistically significant growth above baseline: serine, glycine, and cysteine. Side chain aromaticity, volume, composition, and hydropathy were all significant (p<<0.01) predictors of function, as was overall similarity to the wild-type residue asparagine. Even so, the strengths of these associations were almost entirely driven by the C505 variant: when these data were omitted, the global explanatory power was reduced dramatically (adjusted R²: 0.2263 vs. 0.8664; F-statistic: 9.533 vs. 242). Although some physicochemical properties remained statistically significant predictors of function, the strengths of these associations were generally weak (maximum |Kendall's T|: 0.212).

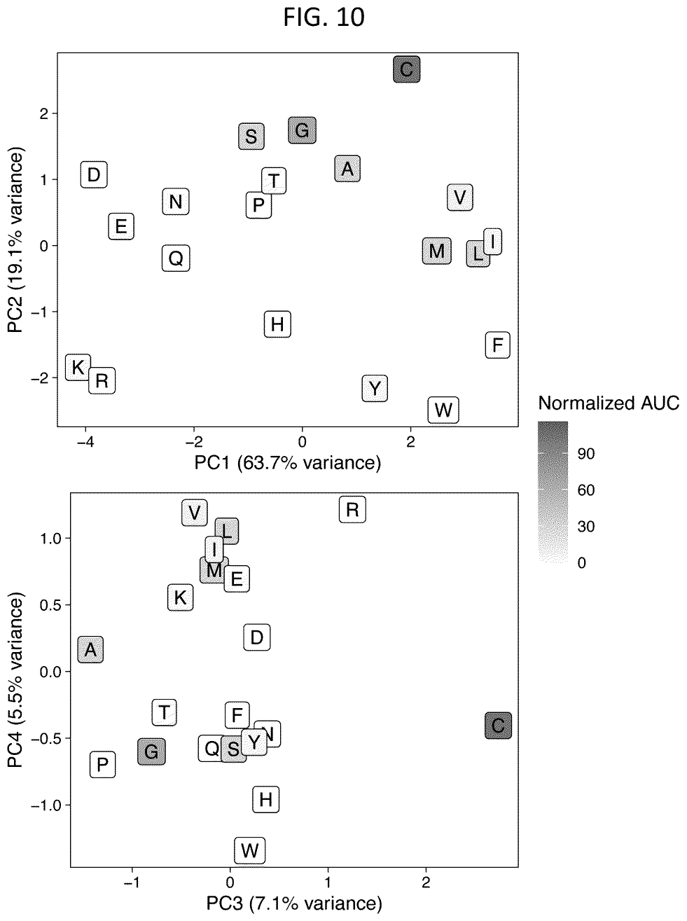

Qualitatively, the fine-scale stringency of physicochemical requirements at position 505 was also noteworthy. Glycine, serine, and cysteine are three of the smallest amino acids, but amino acids with similar side chain volumes did not support growth on maltotriose. Serine and cysteine have side chains of similar size and structure capable of forming hydrogen bonds, but they differ in their polarity and hydrophobicity; nonetheless, residues similar to cysteine in both of these metrics did not support novel function. Indeed, C505's ability to support novel function appeared to be the result of the specific combination of cysteine's physicochemical properties (FIG. 10), albeit not due to its unique capacity to form disulfide bridges (Drew et al. 2021). Remarkably, this effect was dependent on positional context within the transporter: while substituting cysteine to serine at 505 reduced growth by 71.2% (p=8.8×10⁻⁵), making the orthogonal serine to cysteine substitution at another key site, S379 (FIG. 4) reduced growth by 17.7% (p=1.9×10⁻⁶) while still supporting robust growth (FIG. 11). Thus, while serine was largely unable to recapitulate the effect of cysteine at 505, the similarity between the two was sufficient to satisfy the requirements for novel function at position 379. The same was not true of two other hydrogen bond-competent residues, glutamic acid and glutamine, whose introduction at position 379 abolished growth (FIG. 11). This result suggests that, while serine and cysteine are interchangeable at this site, interactions between physical and chemical side chain properties still play a role. Finally, we found further evidence for these fine-scale requirements at position 512, where mutation of the permissive threonine to valine reduced growth by 34.5% (p=7.4×10⁻⁹), while still supporting significantly improved growth over the wild-type MalT4 residue isoleucine (78.1% increase, p=1.2×10⁻⁶). In summary, we find that the strengths, stringencies, and bases of physicochemical requirements all vary between sites that are critical for establishing novel function in MalT434. These results suggest that the serendipitous acquisition of a set of epistatically sufficient residues is highly improbable by point mutations alone (Lynch 2005).

### High-Specificity Transporters are Evolutionarily Derived

The sum of our molecular analyses suggested that the acquisition of novel substrate transport by the high-specificity maltose transporter MalT4 is highly improbable and accessible only through the simultaneous acquisition of numerous interacting substitutions. This observation is consistent with previous failed attempts to establish a maltotriose transporter by introducing as many as 14 rational mutations to S. cerevisiae Mal61 (Hatanaka et al. 2022), a prototypical high-specificity maltose transporter closely related to MalT4. However, the presence of closely related generalist α-glucoside transporters, as typified by S. cerevisiae Agt1, suggests that this ability evolved at least once among yeast α-glucoside transporters. We sought to clarify the timing and mode of this historical evolutionary innovation by examining the phylogenetic relationships between the generalist and specialist α-glucoside transporters within Saccharomycotina yeasts, which have previously been assessed on only a few taxa (Brown et al. 2010; Cousseau et al. 2013; Baker and Hittinger 2019; de Ruijter et al. 2020; Hatanaka et al. 2022; Donzella et al. 2023).

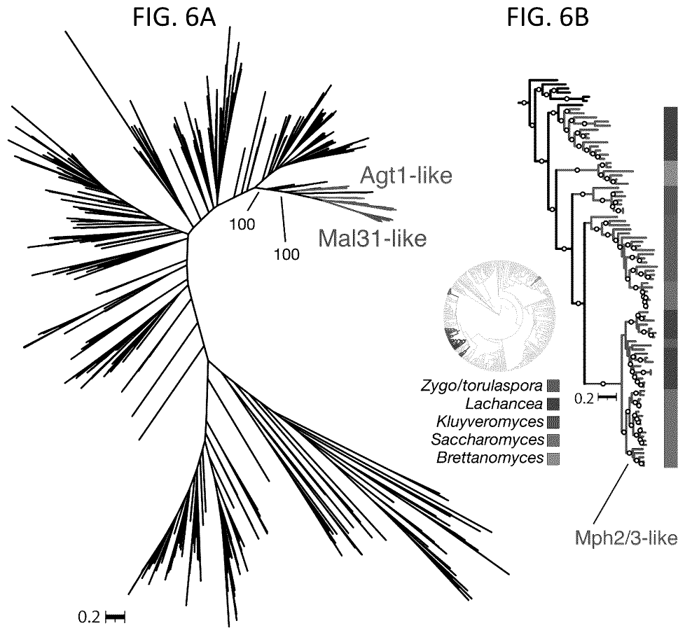

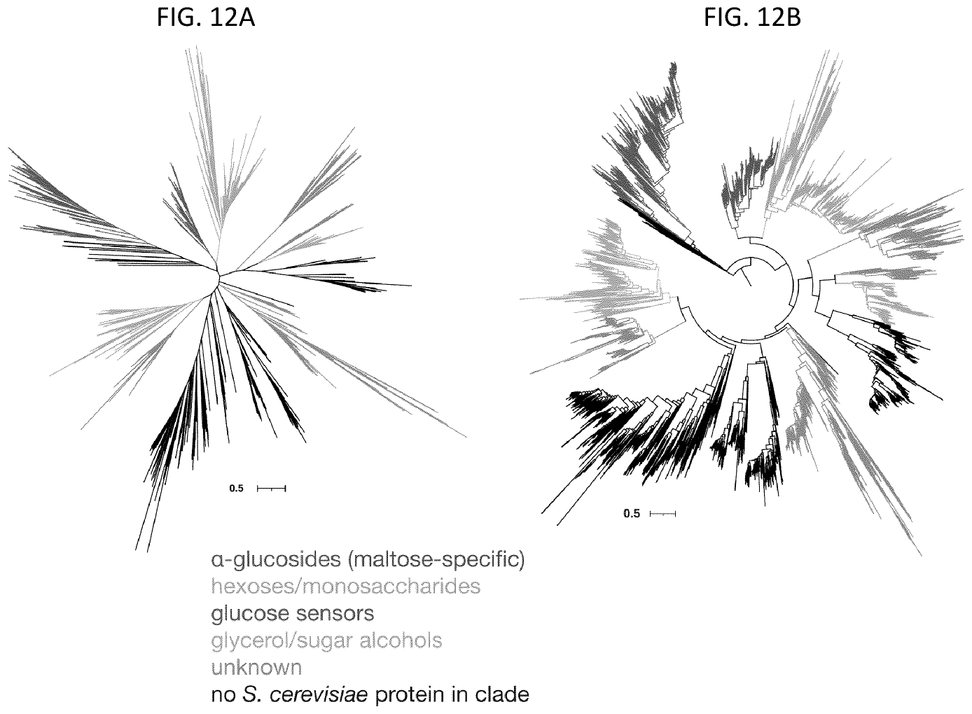

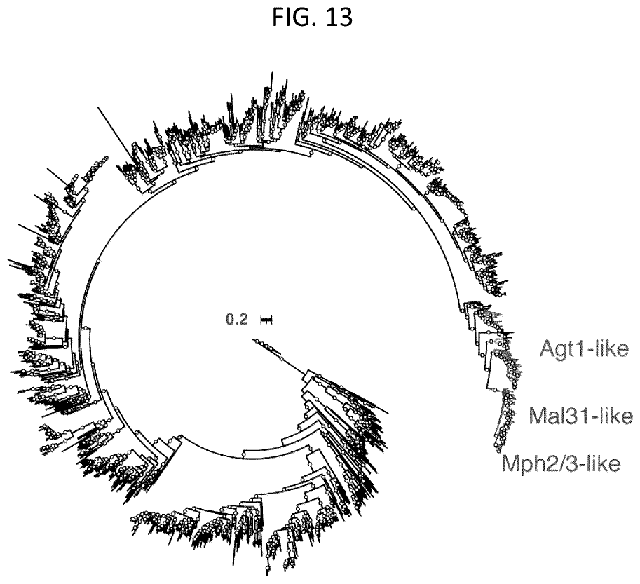

We first generated high-quality protein-coding gene annotations for published genomes from 332 yeast species from the model subphylum Saccharomycotina, which spans more than 400 million years of evolution (X.-X. Shen et al. 2018). To formally test the expected monophyly of the α-glucoside transporters within the broader sugar porter family, we retrieved homologs of S. cerevisiae sugar porters from these predicted proteomes and constructed a comprehensive phylogeny of these 8,403 ecologically and biotechnologically relevant MFS proteins. This phylogeny split into several major clades, many of which contained at least one functionally characterized protein from S. cerevisiae or another species (FIG. 12). Both the high-specificity (Mal31- and Mph2/3-like) and generalist (Agt1-like) α-glucoside transporters clustered in a monophyletic group (“Agt clade”) that excluded other sugar porter families. All proteins in the Agt clade from the newly circumscribed order Saccharomycesles (Groenewald et al. 2023) grouped together with strong support (FIG. 6A). The monophyly of the Saccharomycesles Agts was interrupted in two cases: 1) a single protein from Ogataea naganishii sister to the Lachancea Agt1-like proteins; 2) and, more notably, a well-supported clade of Agts from Brettanomyces anomalus and Brettanomyces bruxellensis. The Brettanomyces species are documented recipients of numerous horizontal gene transfer events, including for genes involved in the metabolism of sucrose, an Agt1 substrate (Stambuk et al. 2000; Woolfit et al. 2007; Roach and Borneman 2020). Notably, B. bruxellensis is commonly associated with brewing environments, where its propensity to vigorously consume diverse sugars and independent evolution of aerobic fermentation make it a frequent contaminant and occasional desired contributor (Rozpedowska et al. 2011; Serra Colomer et al. 2019; Colomer et al. 2020).

Surprisingly, the clade containing high-specificity Saccharomyces maltose transporters only included taxa from closely related species in the genera Saccharomyces and Lachancea, as well as one protein each from Zygotorulaspora florentina and Zygosaccharomyces kombuchaensis (FIG. 6B). Among the high-specificity Agts, the Mph2/3 clade was further restricted to Saccharomyces kudriavzevii, Saccharomyces mikatae, Saccharomyces paradoxus, and S. cerevisiae (FIG. 6B), which is consistent with an origin in the common ancestor of these species following their split from Saccharomyces arboricola and a recent segmental duplication in S. cerevisiae (Saccharomyces jurei is absent from this dataset). The sister clade to the high-specificity proteins contained generalist Agts from Saccharomyces, Torulaspora, and Zygotorulaspora species, with deeper branches to Kluyveromyces and Lachancea homologs (FIG. 6B). We thus conclude that the high-specificity transporters typified by S. cerevisiae Mal31, including S. eubayanus MalT4 and MalT3, form a clade restricted to Saccharomycesles.

Generalist-Like Transporters are Quantitatively Correlated with Growth on α-Glucosides

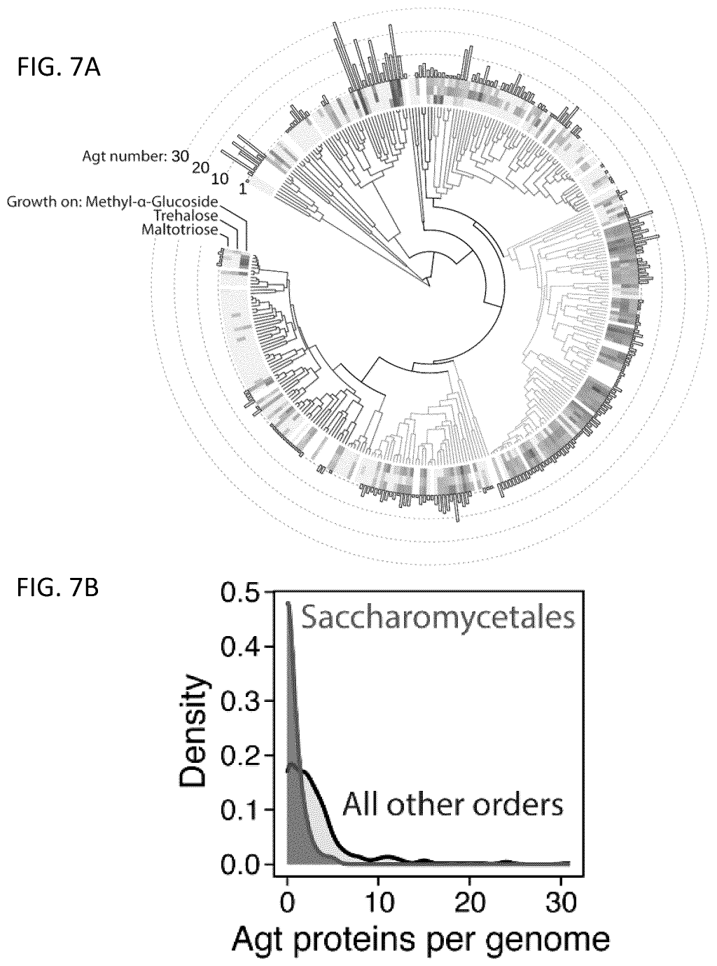

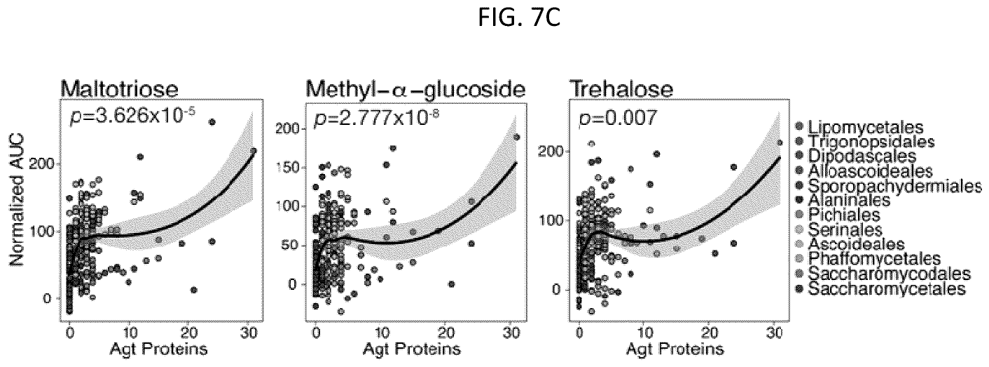

Our phylogenetic analyses suggested that the high-specificity Agts are evolutionarily and functionally derived from a generalist ancestor. In this model, the vast array of uncharacterized Agt-clade proteins encoded by diverse yeast species should include generalist transporters or transporters that became subfunctionalized following duplication of a generalist ancestor, and their presence should support growth on substrates of the generalist Agts. We collected quantitative growth measurements for 287 of the 332 species in our phylogenetic dataset on three sugars that are substrates of the generalist transporter S. cerevisiae Agt1 but not of the high-specificity transporters: maltotriose, trehalose, and methyl-α-glucoside (Han et al. 1995; Stambuk et al. 1999; Stambuk and Araujo 2001; Alves et al. 2008; Brown et al. 2010). We found many species across the Saccharomycotina to be capable of vigorous growth on these sugars as a sole carbon source (FIG. 7A). Growth on all three α-glucosides was nearly ubiquitous among Serinales, a speciose order with a high incidence of carbon niche-breadth generalists (Opulente et al. 2024). Most notably, growth on maltotriose was widespread across the yeast subphylum, in contrast to the documented rarity of this trait in the model genus Saccharomyces (Duval et al. 2010; Gallone et al. 2018; Langdon et al. 2020; Hutzler et al. 2021; Gyurchev et al. 2022; Peris et al. 2023). This metabolic deficiency was concomitant with the paucity of generalist-like Agt proteins encoded in Saccharomycesles genomes, which was similarly not representative of other yeast orders (FIG. 7B; p=1.9×10⁻¹³). Indeed, patterns of α-glucoside growth qualitatively tracked the presence of genes encoding Agt proteins, with both subject to clear evolutionary shifts including losses (e.g., Saccharomycodales, Sporopachydermiales, and Trigonopsidales; Saturnispora, Zygosaccharomyces, Eremothecium, Kazachstania, Nakaseomyces, Naumovozyma, and Tetrapisispora spp.) and amplifications (Debayromyces, Metschnikowia, and Kuraishia spp.; subclades of Phaffomycetales, Dipodascales, Pichiales, and Lipomycetales). We used phylogenetically corrected least squares regressions (PGLSs) to statistically test the strength of the correlation between Agt count and growth on each of the three tested Agt1 substrates (FIG. 7C). We detected significant positive correlations between Agt count and growth on each of the three α-glucosides (p≤0.007). Thus, the generalist-like Agts detected in most Saccharomycotina genomes are likely to be true generalist transporters or recently subfunctionalized derivatives.

### Discussion

In the present work, we sought to understand how novel function could evolve in a model yeast α-glucoside transporter. To this end, we dissected the molecular basis of maltotriose transport in MalT434, which represents one of the most evolutionarily recent functional innovations in this family. We found that, in this chimeric protein, novel function is an emergent property of extensive additive and non-additive interactions between multiple protein regions and multiple residues on TMHs 7, 10, and 11 (FIGS. 2-4). We observed that even conservative amino acid changes, as well as residues not predicted to interact with the substrate, had significant and unexpected effects on maltotriose transport (FIG. 3, FIG. 5). We also found evidence that the stringency of side chain physicochemical requirements likely differs substantially between crucial residues (FIG. 5, FIG. 11). Taken together, these results demonstrate that the evolution of novel function in a high-specificity Agt is highly constrained, which is consistent with recent observations (Hatanaka et al. 2022). In this model, while gene conversion between homologs may not be strictly required for the evolution of novel function in this family, it may indeed be the only remotely probable way that all the necessary interacting residues can readily be assembled in a single molecule, even if paralogs are free to sample neutral or deleterious mutational steps.

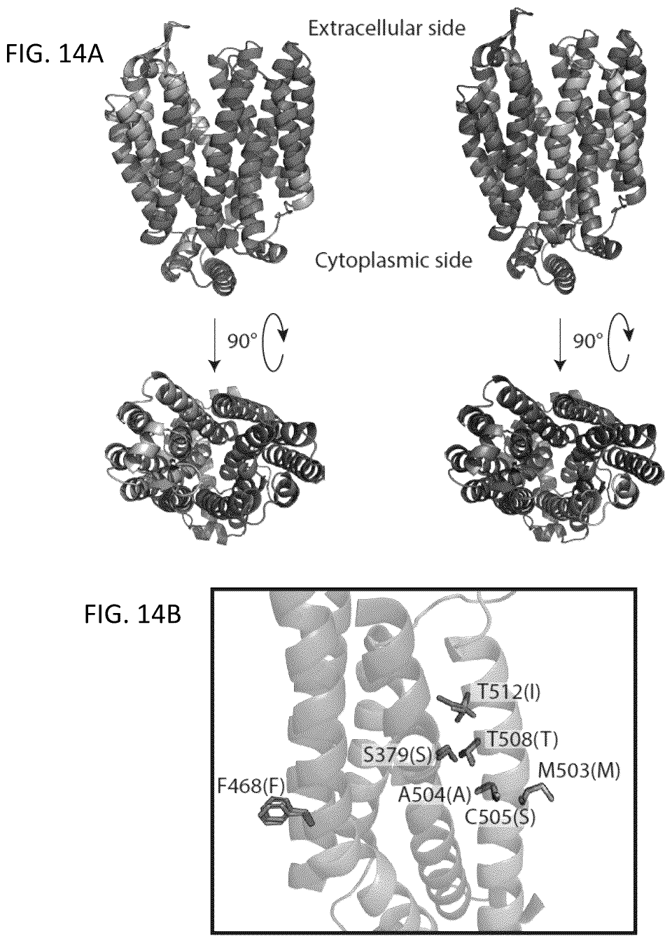

The gene conversion events leading to novel function in high-specificity yeast Agts share striking parallelism at both the sequence and structural scales. For example, the portions of Mty1 inferred to derive from different parental proteins encompass many of the same regions that we identified as having crucial interactions in MalT434 (FIG. 14A). Even more strikingly, the homologous residues at five of the seven sites that affect maltotriose transport in MalT434 are conserved in Mty1 (FIG. 14B). At the other two sites, Mty1 possesses amino acids that support reduced, but significant, growth in MalT434 (C505S and T512I). While many of the same sites likely contribute to novel function in both of these recombinant transporters, specific amino acids at key sites are still likely context-dependent, which makes functional evolution both more difficult to predict and to engineer in this family.

Compounding this difficulty is the cryptic nature of sites that we empirically determined to influence maltotriose transport, but which are unlikely to interact with the substrate (FIG. 1). These substitutions may effect subtle changes to the overall conformation of the transporter, especially where they have the potential to interact with other protein regions that are proximal in tertiary space (e.g., F468). Moreover, there is a growing appreciation that, in yeast monosaccharide sugar porters, the fine-scale environment around the substrate binding site plays a surprisingly large role in sugar recognition and specificity, both by shaping an accommodating binding pocket and through interactions between substrate-interacting and non-interacting residues within van der Waals distance (Kasahara et al. 2009; Drew et al. 2021).

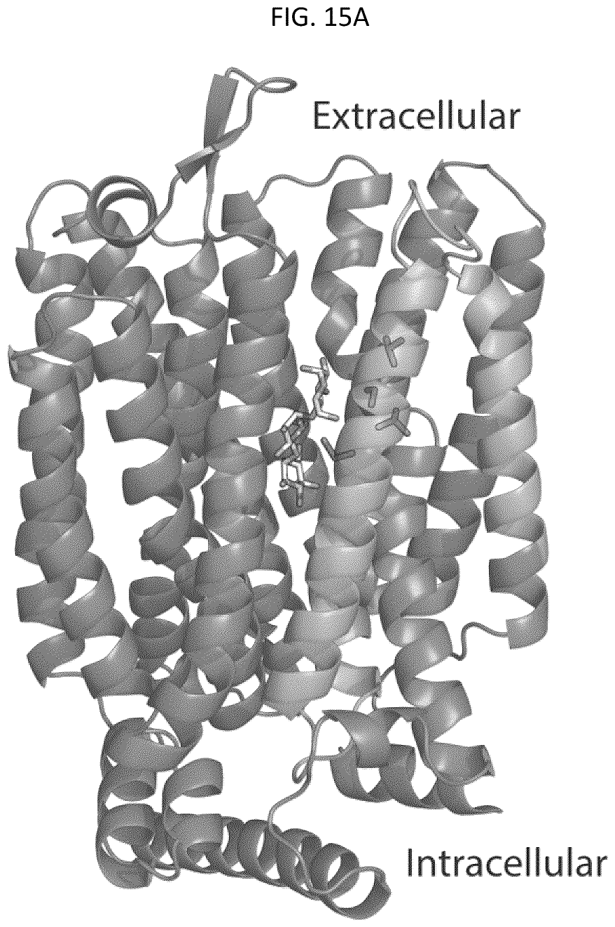

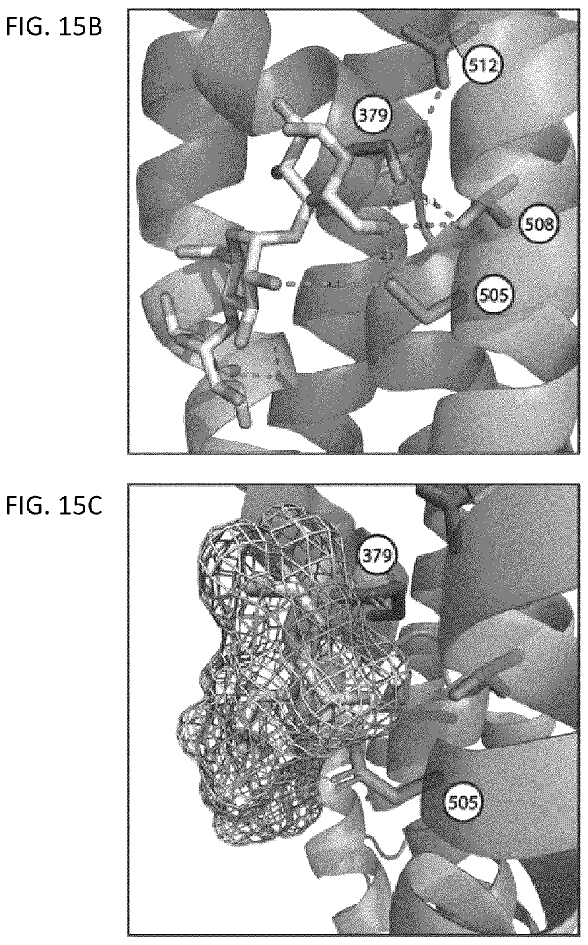

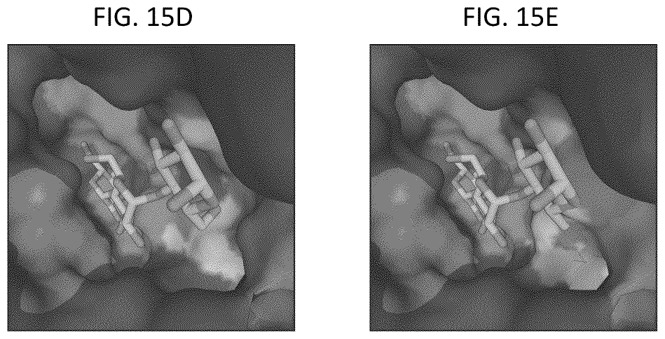

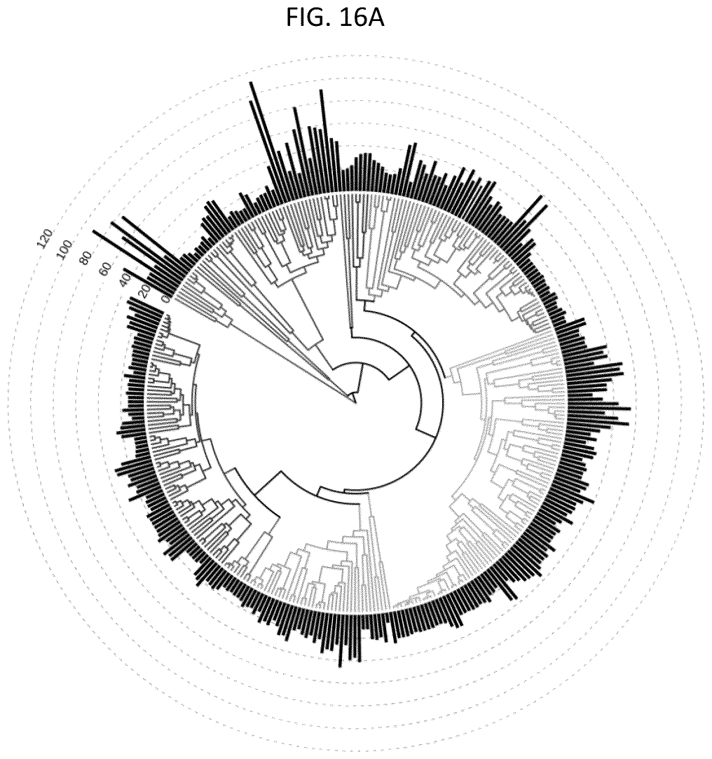

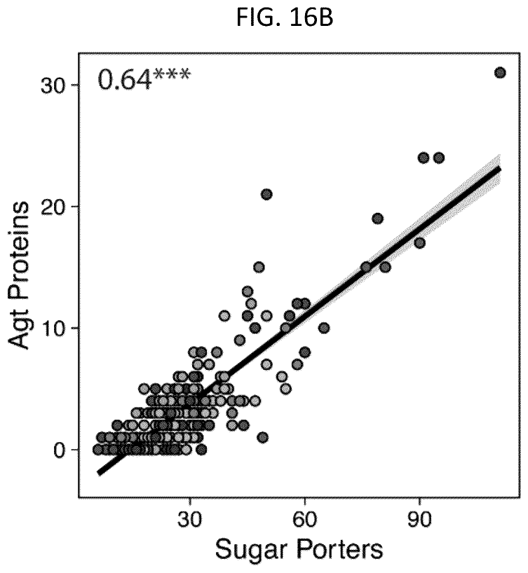

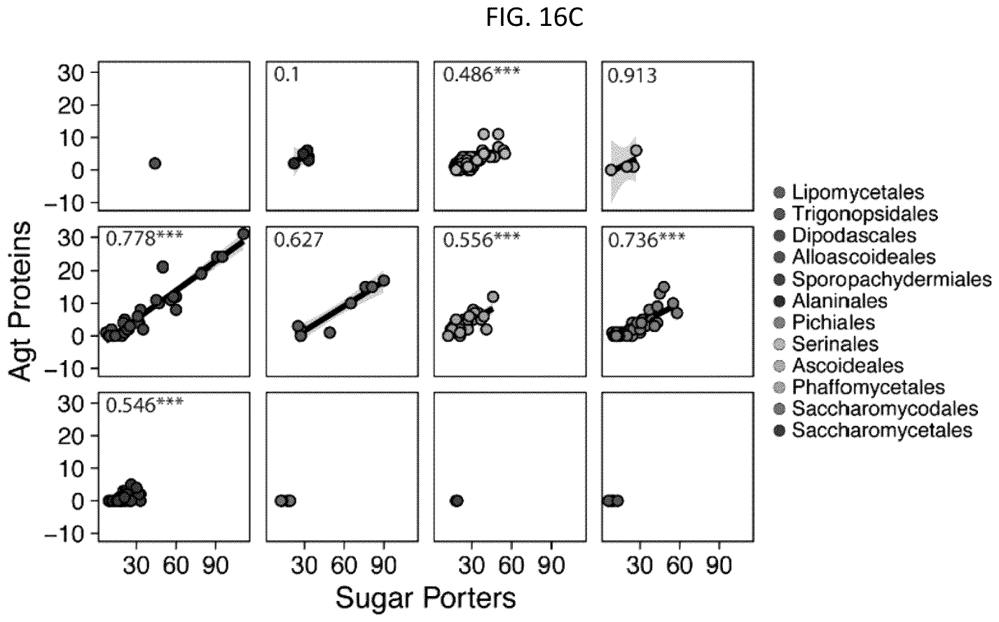

In MalT434, more concrete hypotheses can be made about the molecular contributions of other sites important for novel substrate transport. Molecular docking analyses place the maltotriose ligand in close proximity to the key sites on TMH 7 and TMH 11 (FIG. 15), with several of the sugar hydroxyl groups capable of engaging in a hydrogen-bonding network with the side chains of polar amino acid residues at those sites. Of the substitutions in MalT434 that face the transport channel, all three have polar and hydrogen bond-competent side chains of small-to-medium size; in wild-type MalT4, the residues at these sites have bulkier and/or hydrophobic side chains. Similarly, at the crucial 379 site on TMH 7, the permissive serine has a much smaller side chain than the prohibitive glutamine. Either of the prohibitive residues at 379 and the other crucial site 505 might introduce steric clashes with the terminal glucopyranose moiety of maltotriose (FIG. 15C), even though they themselves are likely capable of hydrogen-bonding with the substrate. Notably, the residue at position 379 may be involved in coupling substrate binding to gating during the transition to the occluded state (Drew et al. 2021), a key determinant of substrate recognition that involves more tightly embedding the sugar molecule in its binding site within the transport channel. In wild-type MalT4, position 379 has the smaller serine residue, while sites along TMH 11 have bulkier amino acids; in wild-type MalT3, position 379 has the larger glutamine residue, but TMH 11 has smaller, hydrophilic residues. Thus, in each native maltose transporter, the steric constraint of the transport channel may be finely tuned at co-evolving sites along TMH 7 and TMH 11 to accommodate maltose with higher affinity and specificity, which occur at the expense of steric exclusion of other substrates, such as maltotriose (FIG. 15E). This model is consistent with the crucial role of amino acid side chain length in shaping substrate specificity in some monosaccharide sugar porters (Kasahara et al. 2011; Drew et al. 2021), notwithstanding that we also detected a complex interaction between size and biochemical properties at the key 505 site.

The difficulty of functional innovation in the high-specificity Agts begs the question of how the related generalist Agts are capable of transporting not only maltose and maltotriose, but a diverse range of substrates. If the generalist transporters had evolved from a more specific ancestor, as has been suggested (Pougach et al. 2014), their extant substrate range would imply multiple bouts of highly constrained functional evolution. To determine when and how this broad substrate specificity may have evolved in the generalist Agts, we reconstructed the yeast sugar porter phylogeny from 332 newly annotated, representative Saccharomycotina genomes encompassing more than 400 million years of evolution (FIG. 12). This analysis showed that, somewhat unexpectedly, the high-specificity Agts are a derived clade within the generalist-like Agts (FIG. 6A). The copy number of these putative generalist Agts encoded by yeast genomes is strongly predictive of growth on Agt1-exclusive substrates (FIG. 7), which further supports the conclusion that these proteins are likely bona fide generalists. The evolution of maltotriose transport by high-specificity Agts is thus better regarded as a reacquisition of ancestral function than the de novo evolution of a truly novel function within this protein family.

It remains subject to debate whether the general trend of protein evolution is directional: from less to more intrinsically specific (Bridgham et al. 2006; Tawfik 2010; Copley 2012; Steindel et al. 2016; Wheeler et al. 2016; Wheeler and Harms 2021). Multiple lines of evidence now suggest that this mode predominates among genes involved in α-glucoside metabolism in yeasts. In addition to the α-glucoside transporters, both the α-glucosidases of S. cerevisiae and the transcriptional activators that regulate the structural metabolic genes likely evolved from promiscuous ancestral proteins that optimized subfunctions following duplication events, rendering them specific for different α-glucosides (Brown et al. 2010; Voordeckers et al. 2012; Pougach et al. 2014). The extent of intramolecular epistasis apparent in the high-specificity Agts, which may arise both from intra-protein and protein-substrate interactions, may provide an explanation for the inherent difficulty of re-evolving maltotriose transport in these proteins. Functional entrenchment by historical contingency and epistasis is well documented, and the irreversibility of evolutionary trajectories at the molecular level may be a widespread phenomenon (Ortlund et al. 2007; Bridgham et al. 2009; Soylemez and Kondrashov 2012; Harms and Thornton 2014; Bank et al. 2015; Podgornaia and Laub 2015; Shah et al. 2015; Starr and Thornton 2016; Starr et al. 2017; Starr et al. 2018; Ben-David et al. 2020; Xie et al. 2021; Park et al. 2022). Although not directly tested here, there may be inherent tradeoffs between specificity and substrate affinity in yeast Agts (Stambuk and Araujo 2001; Salema-Oom et al. 2005; Hatanaka et al. 2022), which would suggest that walking back through the accumulated mutations that led to higher specificity in the Mal31-like transporters would be likely to incur an immediate functional tradeoff and therefore fitness cost. The recurrent gene conversion events that enable maltotriose transport among members of this family may, therefore, represent the only meaningfully accessible route to bypass these deleterious intermediates, but the high degree of context-dependence for mutational effects makes the prediction or engineering of this novel function difficult (Hatanaka et al. 2022).

Might the evolution of yeast sugar porters more broadly be organized along an axis of increasing specialization and specificity? This family encompasses functionally diverse transporters with varying specificities for different mono- and di-saccharides and sugar alcohols; notably, functionally similar proteins are not monophyletic across the family (Donzella et al. 2023). Our phylogenetic analysis of these proteins places the Agts, which may retain some glucose transport capacity (Wieczorke et al. 1999), as a deeply branching sister clade to most of the broader family (FIG. 12). These results imply multiple bouts of functional specialization from a highly promiscuous ancestor, in some cases starting from partially subfunctionalized ancestral proteins, with the Agts perhaps remaining the most representative of the ancestral multifunctionality. Supporting this notion, the Agt gene family is a key contributor to the carbon niche breadth of more than 1,000 yeasts (David et al. 2024). While the extant diversity of yeast sugar porters has generally been regarded as an example of functional diversification (i.e., highly plastic gains of novel substrate affinity; (Brown et al. 2010; Hatanaka et al. 2022; Donzella et al. 2023)), the evolution of this important gene family may have followed a very different mode. In the former model, functional diversification by neofunctionalization follows duplication of ancestral transporter genes, whereas our analyses suggest that duplications in this gene family may be primarily followed by subfunctionalizing escapes from adaptive conflict (Hughes 1994; Hittinger and Carroll 2007; Des Marais and Rausher 2008), wherein transporters can gain increased specificity and affinity for a narrow substrate range at the expense of other ancestral ligands.

These two models have distinct implications for the myriad biotechnological applications predicated upon sugar consumption by yeasts, which might be targets for improvement by protein engineering. If extant transporters are indeed highly plastic and evolvable, shifting or expanding their substrate range should be relatively simple. If, on the other hand, they have undergone entrenched specialization, they may be inherently less evolvable (Bridgham et al. 2009; Starr et al. 2018; Wheeler and Harms 2021). Results here and elsewhere (Hatanaka et al. 2022) support the latter corollary. However, this model also implies that reconstructed ancestral proteins, or even generalist extant proteins from this clade, might both possess desirable properties and be inherently highly amenable to engineering, mutagenesis, or directed evolution approaches.

### Materials and Methods:

### Strains and Cultivation Conditions

S. eubayanus strains, plasmids, and oligonucleotides used in this work are listed in Table 2 and Table 3. Yeasts were propagated on YPD medium (1% yeast extract, 2% peptone, 2% glucose) supplemented with 400 mg/L G418 and/or 50 mg/L Nourseothricin (CloNAT) as appropriate and cryopreserved in 15% glycerol at −80° for long-term storage.

TABLE 2
S. eubayanus strains and plasmids used in this study

| Strain ID | Genotype | Derived from | Reference |
|---|---|---|---|
| yHKS210 | Wild strain | | Peris et al. |
| | | | 2014 |
| yHEB1505 | MATα/MATa MALT434/MALT434 | yHKS210 | Baker and |
| | | | Hittinger |
| | | | 2019 |
| yHJC186 | MATα/MATa malt2Δ::kanMX/malt2Δ::kanMX | yHKS210 | This study |
| | malt4Δ::kanMX/malt4Δ::kanMX | | |
| yHJC195 | MATα/MATa ho-Δ/ho-Δ | yHKS210 | This study |
| yHJC200 | MATα/MATa malt2Δ::kanMX/malt2Δ::kanMX | yHJC186 | This study |
| | malt4Δ::kanMX/malt4Δ::kanMX ho-Δ/ho-Δ | | |
| yHJC207 | MATα malt2Δ::kanMX malt4Δ::kanMX ho-Δ | yHJC200 | This study |
| yHJC211 | MATα ho-Δ | yHJC195 | This study |
| yHJC233 | MATα malt2Δ::Chimera9 malt4Δ::Chimera9 ho-Δ | yHJC207 | This study |
| yHJC234 | MATα malt2Δ::Chimera10 malt4Δ::Chimera10 ho-Δ | yHJC207 | This study |
| yHJC236 | MATα malt2Δ::Chimera17 malt4Δ::Chimera17 ho-Δ | yHJC207 | This study |
| yHJC237 | MATα malt2Δ::Chimera1 malt4Δ::Chimera1 ho-Δ | yHJC207 | This study |
| yHJC245 | MATα malt2Δ::MALT4^{S468F I503M G504A N505C V508T I512T} | yHJC207 | This study |
| | malt4Δ::MALT4^{S468F I503M G504A N505C V508T I512T} ho-Δ | | |
| yHJC246 | MATα malt2Δ::MALT4^{S468F I503M G504A N505C V508T I512T} | yHJC207 | This study |
| | malt4Δ::MALT4^{S468F I503M G504A N505C V508T I512T} ho-Δ | | |
| yHJC247 | MATα malt2Δ::MALT4^{S468F I503M G504A N505C V508T I512T} | yHJC207 | This study |
| | malt4Δ::MALT4^{S468F I503M G504A N505C V508T I512T} ho-Δ | | |
| yHJC248 | MATα malt2Δ::MALT4^{S468F I503M G504A N505C V508T I512T} | yHJC207 | This study |
| | malt4Δ::MALT4^{S468F I503M G504A N505C V508T I512T} ho-Δ | | |
| yHJC249 | MATα malt2Δ::MALT4^{N522D F534L L536F V538T I540V} | yHJC207 | This study |
| | malt4Δ::MALT4^{N522D F534L L536F V538T I540V} ho-Δ | | |
| yHJC250 | MATα malt2Δ::MALT4^{N522D F534L L536F V538T I540V} | yHJC207 | This study |
| | malt4Δ::MALT4^{N522D F534L L536F V538T I540V} ho-Δ | | |
| yHJC251 | MATα malt2Δ::MALT4^{N522D F534L L536F V538T I540V} | yHJC207 | This study |
| | malt4Δ::MALT4^{N522D F534L L536F V538T I540V} ho-Δ | | |
| yHJC252 | MATα malt2Δ::MALT4^{N522D F534L L536F V538T I540V} | yHJC207 | This study |
| | malt4Δ::MALT4^{N522D F534L L536F V538T I540V} ho-Δ | | |
| yHJC253 | MATα malt2Δ::MALT4^{S468F N522D} malt4Δ::MALT4^{S468F N522D} ho-Δ | yHJC207 | This study |
| yHJC254 | MATα malt2Δ::MALT4^{S468F N522D} malt4Δ::MALT4^{S468F N522D} ho-Δ | yHJC207 | This study |
| yHJC255 | MATα malt2Δ::MALT4^{S468F N522D} malt4Δ::MALT4^{S468F N522D} ho-Δ | yHJC207 | This study |
| yHJC256 | MATα malt2Δ::MALT4^{S468F N522D} malt4Δ::MALT4^{S468F N522D} ho-Δ | yHJC207 | This study |
| yHJC283 | MATα malt2Δ::MALT434 malt4Δ::MALT434 ho-Δ | yHJC207 | This study |
| yHJC284 | MATα malt2Δ::sseAGT1 malt4Δ::seAGTI ho-Δ | yHJC207 | This study |
| yHJC285 | MATα malt2Δ::sseAGT1 malt4Δ::seAGT1 ho-Δ | yHJC207 | This study |
| yHJC286 | MATα malt2Δ::Chimera11 malt4Δ::Chimerall ho-Δ | yHJC207 | This study |
| yHJC307 | MATα malt2Δ::MALT4^{S468F I503M G504A} malt4A::MALT4^{S468F I503M G504A} | yHJC207 | This study |
| | ho-Δ | | |
| yHJC308 | MATα malt2Δ::MALT4^{S468F I503M G504A} malt4Δ::MALT4^{S468F I503M G504A} | yHJC207 | This study |
| | ho-Δ | | |
| yHJC309 | MATα malt2A:MALT4^{I503M G504A N505C} malt4Δ::MALT4^{I503M G504A N505C} | yHJC207 | This study |
| | ho-Δ | | |
| yHJC312 | MATα malt2Δ::MALT4^{N505C V508T I512T} malt4Δ::MALT4^{N505C V508T I512T} | yHJC207 | This study |
| | ho-Δ | | |
| yHJC313 | MATα malt2Δ::MALT4^{N505C V508T I512T} malt4Δ::MALT4^{N505C V508T I512T} | yHJC207 | This study |
| | ho-Δ | | |
| yHJC319 | MATα malt2Δ::MALT4^{I503M G504A N505C} malt4Δ::MALT4^{I503M G504A N505C} | yHJC207 | This study |
| | ho-Δ | | |
| yHJC320 | MATα malt2Δ::MALT4^{I503M G504A N505C} malt4Δ::MALT4^{I503M G504A N505C} | yHJC207 | This study |
| | ho-Δ | | |
| yHJC321 | MATα malt2Δ::Chimera1 malt4Δ::Chimera1 ho-Δ | yHJC207 | This study |
| yHJC323 | MATα malt2Δ::MALT4^{I503M G504A N505C} malt4Δ::MALT4^{I503M G504A N505C} | yHJC207 | This study |
| | ho-Δ | | |
| yHJC330 | MATα malt2Δ::MALT4^{I503M G504A N505C V508T I512T} | yHJC207 | This study |
| | malt4Δ::MALT4^{I503M G504A N505C V508T I512T} ho-Δ | | |
| yHJC331 | MATα malt2Δ::MALT4^{I503M G504A N505C V508T I512T} | yHJC207 | This study |
| | malt4Δ::MALT4^{I503M G504A N505C V508T I512T} ho-Δ | | |
| yHJC332 | MATα malt2Δ::MALT4^{I503M G504A N505C V508T I512T} | yHJC207 | This study |
| | malt4Δ::MALT4^{I503M G504A N505C V508T I512T} ho-Δ | | |
| yHJC333 | MATα malt2Δ::MALT4^{S468F G504A N505C V508T I512T} | yHJC207 | This study |
| | malt4Δ::MALT4^{S468F G504A N505C V508T I512T} ho-Δ | | |
| yHJC334 | MATα malt2Δ::MALT4^{S468F G504A N505C V508T I512T} | yHJC207 | This study |
| | malt4Δ::MALT4^{S468F G504A N505C V508T I512T} ho-Δ | | |
| yHJC335 | MATα malt2Δ::MALT4^{S468F G504A N505C V508T I512T} | yHJC207 | This study |
| | malt4Δ::MALT4^{S468F G504A N505C V508T I512T} ho-Δ | | |
| yHJC336 | MATα malt2Δ::MALT4^{S468F G504A N505C V508T I512T} | yHJC207 | This study |
| | malt4Δ::MALT4^{S468F G504A N505C V508T I512T} ho-Δ | | |
| yHJC339 | MATα malt2Δ::MALT4^{S468F I503M N505C V508T I512T} | yHJC207 | This study |
| | malt4Δ::MALT4^{S468F I503M N505C V508T I512T} ho-Δ | | |
| yHJC340 | MATα malt2Δ::MALT4^{S468F I503M N505C V508T I512T} | yHJC207 | This study |
| | malt4Δ::MALT4^{S468F I503M N505C V508T I512T} ho-Δ | | |
| yHJC341 | MATα malt2Δ::MALT4^{S468F I503M N505C V508T I512T} | yHJC207 | This study |
| | malt4Δ::MALT4^{S468F I503M N505C V508T I512T} ho-Δ | | |
| yHJC342 | MATα malt2Δ::MALT4^{S468F I503M G504A V508T I512T} | yHJC207 | This study |
| | malt4Δ::MALT4^{S468F I503M G504A V508T I512T} ho-Δ | | |
| yHJC343 | MATα malt2Δ::MALT4^{S468F I503M G504A V508T I512T} | yHJC207 | This study |
| | malt4Δ::MALT4^{S468F I503M G504A V508T I512T} ho-Δ | | |
| yHJC344 | MATα malt2Δ::MALT4^{S468F I503M G504A N505C I512T} | yHJC207 | This study |
| | malt4Δ::MALT4^{S468F I503M G504A N505C I512T} ho-Δ | | |
| yHJC345 | MATα malt2Δ::MALT4^{S468F I503M G504A N505C I512T} | yHJC207 | This study |
| | malt4Δ::MALT4^{S468F I503M G504A N505C I512T} ho-Δ | | |
| yHJC346 | MATα malt2Δ::MALT4^{S468F I503M G504A N505C I512T} | yHJC207 | This study |
| | malt4Δ::MALT4^{S468F I503M G504A N505C I512T} ho-Δ | | |
| yHJC348 | MATα malt2Δ::MALT4^{S468F I503M G504A N505C V508T} | yHJC207 | This study |
| | malt4Δ::MALT4^{S468F I503M G504A N505C V508T} ho-Δ | | |
| yHJC349 | MATα malt2Δ::MALT4^{S468F I503M G504A N505C V508T} | yHJC207 | This study |
| | malt4Δ::MALT4^{S468F I503M G504A N505C V508T} ho-Δ | | |
| yHJC350 | MATα malt2Δ::MALT4^{S468F I503M G504A N505C V508T} | yHJC207 | This study |
| | malt4Δ::MALT4^{S468F I503M G504A N505C V508T} ho-Δ | | |
| yHJC409 | MATα malt2Δ::Chimera8 malt4Δ::Chimera8 ho-Δ | yHJC207 | This study |
| yHJC410 | MATα malt2Δ::Chimera5 malt4Δ::Chimera5 ho-Δ | yHJC207 | This study |
| yHJC411 | MATα malt2Δ::Chimera2 malt4Δ::Chimera2 ho-Δ | yHJC207 | This study |
| yHJC412 | MATα malt2Δ::Chimera2 malt4Δ::Chimera2 ho-Δ | yHJC207 | This study |
| yHJC417 | MATα malt2Δ::MALT4^{I503M N505C I512T} malt4Δ::MALT4^{I503M N505C I512T} | yHJC207 | This study |
| | ho-Δ | | |
| yHJC423 | MATα malt2Δ::Chimera8 malt4Δ::Chimera8 ho-Δ | yHJC207 | This study |
| yHJC424 | MATα malt2Δ::Chimera8 malt4Δ::Chimera8 ho-Δ | yHJC207 | This study |
| yHJC425 | MATα malt2Δ::Chimera8 malt4Δ::Chimera8 ho-Δ | yHJC207 | This study |
| yHJC426 | MATα malt2Δ::Chimera12 malt4Δ::Chimera12 ho-Δ | yHJC207 | This study |
| yHJC427 | MATα malt2Δ::Chimera5 malt4Δ::Chimera5 ho-Δ | yHJC207 | This study |
| yHJC428 | MATα malt2Δ::Chimera5 malt4Δ::Chimera5 ho-Δ | yHJC207 | This study |
| yHJC429 | MATα malt2Δ::Chimera5 malt4Δ::Chimera5 ho-Δ | yHJC207 | This study |
| yHJC430 | MATα malt2Δ::MALT4^{I503M N505C V508T I512T} malt4Δ::MALT4^{I503M N505C V508T I512T} | yHJC207 | This study |
| | ho-Δ | | |
| yHJC431 | MATα malt2Δ::MALT4^{I503M N505C V508T I512T} malt4Δ::MALT4^{I503M N505C V508T I512T} | yHJC207 | This study |
| | ho-Δ | | |
| yHJC432 | MATα malt2Δ::MALT4^{I503M N505C V508T I512T} malt4Δ::MALT4^{I503M N505C V508T I512T} | yHJC207 | This study |
| | ho-Δ | | |
| yHJC433 | MATα malt2Δ::MALT4^{I503M N505C I512T} malt4Δ::MALT4^{I503M N505C I512T} | yHJC207 | This study |
| | ho-Δ | | |
| yHJC434 | MATα malt2Δ::MALT4^{I503M N505C I512T} malt4Δ::MALT4^{I503M N505C I512T} | yHJC207 | This study |
| | ho-Δ | | |
| yHJC487 | MATα malt2Δ::Chimera16 malt4Δ::Chimera16 ho-Δ | yHJC207 | This study |
| yHJC518 | MATα malt2Δ::Chimera3 malt4Δ::Chimera3 ho-Δ | yHJC207 | This study |
| yHJC526 | MATα malt2Δ::Chimera4 malt4Δ::Chimera4 ho-Δ | yHJC207 | This study |
| yHJC533 | MATα malt2Δ::Chimera6 malt4Δ::Chimera6 ho-Δ | yHJC207 | This study |
| yHJC534 | MATα malt2Δ::Chimera6 malt4Δ::Chimera6 ho-Δ | yHJC207 | This study |
| yHJC535 | MATα malt2Δ::Chimera7 malt4Δ::Chimera7 ho-Δ | yHJC207 | This study |
| yHJC536 | MATα malt2Δ::Chimera7 malt4Δ::Chimera7 ho-Δ | yHJC207 | This study |
| yHJC537 | MATα malt2Δ::Chimera7 malt4Δ::Chimera7 ho-Δ | yHJC207 | This study |
| yHJC538 | MATα malt2Δ::Chimera7 malt4Δ::Chimera7 ho-Δ | yHJC207 | This study |
| yHJC539 | MATα malt2Δ::MALT4^{A378T S379Q S468F I503M G504A N505C V508T I512T} | yHJC207 | This study |
| | malt4Δ::MALT4^{A378T S379Q S468F I503M G504A N505C V508T I512T} ho-Δ | | |
| yHJC545 | MATα malt2Δ::MALT4^{S379C S468F I503M G504A N505C V508T I512T} | yHJC207 | This study |
| | malt4Δ::MALT4^{S379C S468F I503M G504A N505C V508T I512T} ho-Δ | | |
| yHJC546 | MATα malt2Δ::MALT4^{S379C S468F I503M G504A N505C V508T I512T} | yHJC207 | This study |
| | malt4Δ::MALT4^{S379C S468F I503M G504A N505C V508T I512T} ho-Δ | | |
| yHJC547 | MATα malt2Δ::MALT4^{S379C S468F I503M G504A N505C V508T I512T} | yHJC207 | This study |
| | malt4Δ::MALT4^{S379C S468F I503M G504A N505C V508T I512T} ho-Δ | | |
| yHJC548 | MATα malt2Δ::MALT4^{S379E S468F I503M G504A N505C V508T I512T} | yHJC207 | This study |
| | malt4Δ::MALT4^{S379E S468F I503M G504A N505C V508T I512T} ho-Δ | | |
| yHJC549 | MATα malt2Δ::MALT4^{S379E S468F I503M G504A N505C V508T I512T} | yHJC207 | This study |
| | malt4Δ::MALT4^{S379E S468F I503M G504A N505C V508T I512T} ho-Δ | | |
| yHJC550 | MATα malt2Δ::MALT4^{S468F I503M G504A N505D V508T I512T} | yHJC207 | This study |
| | malt4Δ::MALT4^{S468F I503M G504A N505D V508T I512T} ho-Δ | | |
| yHJC551 | MATα malt2Δ::MALT4^{S468F I503M G504A N505Q V508T I512T} | yHJC207 | This study |
| | malt4Δ::MALT4^{S468F I503M G504A N505Q V508T I512T} ho-Δ | | |
| yHJC552 | MATα malt2Δ::MALT4^{S468F I503M G504A N505C V508T I512V} | yHJC207 | This study |
| | malt4Δ::MALT4^{S468F I503M G504A N505C V508T I512V} ho-Δ | | |
| yHJC553 | MATα malt2Δ::MALT4^{S468F I503M G504A N505C V508T I512V} | yHJC207 | This study |
| | malt4Δ::MALT4^{S468F I503M G504A N505C V508T I512V} ho-Δ | | |
| yHJC554 | MATα malt2Δ::MALT4^{S468F I503M G504A N505C V508T I512V} | yHJC207 | This study |
| | malt4Δ::MALT4^{S468F I503M G504A N505C V508T I512V} ho-Δ | | |
| yHJC555 | MATα malt2Δ::Chimera13 malt4Δ::Chimera13 ho-Δ | yHJC207 | This study |
| yHJC556 | MATα malt2Δ::Chimera13 malt4Δ::Chimera13 ho-Δ | yHJC207 | This study |
| yHJC557 | MATα malt2Δ::Chimera13 malt4Δ::Chimera13 ho-Δ | yHJC207 | This study |
| yHJC560 | MATα malt2Δ::Chimera15 malt4Δ::Chimera15 ho-Δ | yHJC207 | This study |
| yHJC561 | MATα malt2Δ::Chimera15 malt4Δ::Chimera15 ho-Δ | yHJC207 | This study |
| yHJC562 | MATα malt2Δ::Chimera15 malt4Δ::Chimera15 ho-Δ | yHJC207 | This study |
| yHJC563 | MATα malt2Δ::Chimera14 malt4Δ::Chimera14 ho-Δ | yHJC207 | This study |
| yHJC564 | MATα malt2Δ::Chimera14 malt4Δ::Chimera14 ho-Δ | yHJC207 | This study |
| yHJC565 | MATα malt2Δ::MALT4^{A378T S468F I503M G504A N505C V508T I512T} | yHJC207 | This study |
| | malt4Δ::MALT4^{A378T S468F I503M G504A N505C V508T I512T} ho-Δ | | |
| yHJC566 | MATα malt2Δ::MALT4^{S379Q S468F I503M G504A N505C V508T I512T} | yHJC207 | This study |
| | malt4Δ::MALT4^{S379Q S468F I503M G504A N505C V508T I512T} ho-Δ | | |
| yHJC567 | MATα malt2Δ::Chimera14 malt4Δ::Chimera14 ho-Δ | yHJC207 | This study |
| yHJC568 | MATα malt2Δ::MALT4^{A378T S468F I503M G504A N505C V508T I512T} | yHJC207 | This study |
| | malt4Δ::MALT4^{A378T S468F I503M G504A N505C V508T I512T} ho-Δ | | |
| yHJC569 | MATα malt2Δ::MALT4^{A378T S468F I503M G504A N505C V508T I512T} | yHJC207 | This study |
| | malt4Δ::MALT4^{A378T S468F I503M G504A N505C V508T I512T} ho-Δ | | |
| yHJC573 | MATα malt2Δ::Chimera14 malt4Δ::Chimera14 ho-Δ | yHJC207 | This study |
| yHJC574 | MATα malt2Δ::MALT4^{S379Q S468F I503M G504A N505C V508T I512T} | yHJC207 | This study |
| | malt4Δ::MALT4^{S379Q S468F I503M G504A N505C V508T I512T} ho-Δ | | |
| yHJC578 | MATα malt2Δ::MALT4^{S379Q S468F I503M G504A N505E V508T I512T} | yHJC207 | This study |
| | malt4Δ::MALT4^{S379Q S468F I503M G504A N505E V508T I512T} ho-Δ | | |
| yHJC581 | MATα malt2Δ::MALT4^{S3790 S468F I503M G504A N505G V508T I512T} | yHJC207 | This study |
| | malt4Δ::MALT4^{S379Q S468F I503M G504A N505G V508T I512T} ho-Δ | | |
| yHJC582 | MATα malt2Δ::MALT4^{S379Q S468F I503M G504A N505F V508T I512T} | yHJC207 | This study |
| | malt4Δ::MALT4^{S379Q S468F I503M G504A N505F V508T I512T} ho-Δ | | |
| yHJC583 | MATα malt2Δ::MALT4^{S379Q S468F I503M G504A N505I V508T I512T} | yHJC207 | This study |
| | malt4Δ::MALT4^{S379Q S468F I503M G504A N505I V508T I512T} ho-Δ | | |
| yHJC584 | MATα malt2Δ::MALT4^{S379Q S468F I503M G504A N505R V508T I512T} | yHJC207 | This study |
| | malt4Δ::MALT4^{S379Q S468F I503M G504A N505R V508T I512T} ho-Δ | | |
| yHJC585 | MATα malt2Δ::MALT4^{S379Q S468F I503M G504A N505P V508T I512T} | yHJC207 | This study |
| | malt4Δ::MALT4^{S379Q S468F I503M G504A N505P V508T I512T} ho-Δ | | |
| yHJC586 | MATα malt2Δ::MALT4^{S379Q S468F I503M G504A N505E V508T I512T} | yHJC207 | This study |
| | malt4Δ::MALT4^{S379Q S468F I503M G504A N505E V508T I512T} ho-Δ | | |
| yHJC587 | MATα malt2Δ::MALT4^{S379Q S468F I503M G504A N505R V508T I512T} | yHJC207 | This study |
| | malt4Δ::MALT4^{S379Q S468F I503M G504A N505R V508T I512T} ho-Δ | | |
| yHJC588 | MATα malt2Δ::MALT4^{S379Q S468F I503M G504A N505W V508T I512T} | yHJC207 | This study |
| | malt4Δ::MALT4^{S379Q S468F I503M G504A N505W V508T I512T} ho-Δ | | |
| yHJC589 | MATα malt2Δ::MALT4^{S379Q S468F I503M G504A N505K V508T I512T} | yHJC207 | This study |
| | malt4Δ::MALT4^{S379Q S468F I503M G504A N505K V508T I512T} ho-Δ | | |
| yHJC590 | MATα malt2Δ::MALT4^{S379Q S468F I503M G504A N505P V508T I512T} | yHJC207 | This study |
| | malt4Δ::MALT4^{S379Q S468F I503M G504A N505P V508T I512T} ho-Δ | | |
| yHJC591 | MATα malt2Δ::MALT4^{S379Q S468F I503M G504A N505H V508T I512T} | yHJC207 | This study |
| | malt4Δ::MALT4^{S379Q S468F I503M G504A N505H V508T I512T} ho-Δ | | |
| yHJC592 | MATα malt2Δ::MALT4^{S379Q S468F I503M G504A N505K V508T I512T} | yHJC207 | This study |
| | malt4Δ::MALT4^{S379Q S468F I503M G504A N505K V508T I512T} ho-Δ | | |
| yHJC593 | MATα malt2Δ::MALT4^{S379Q S468F I503M G504A N505V V508T I512T} | yHJC207 | This study |
| | malt4Δ::MALT4^{S379Q S468F I503M G504A N505V V508T I512T} ho-Δ | | |
| yHJC594 | MATα malt2Δ::MALT4^{S379Q S468F I503M G504A N505K V508T I512T} | yHJC207 | This study |
| | malt4Δ::MALT4^{S379Q S468F I503M G504A N505K V508T I512T} ho-Δ | | |
| yHJC595 | MATα malt2Δ::MALT4^{S379Q S468F I503M G504A N505Y V508T I512T} | yHJC207 | This study |
| | malt4Δ::MALT4^{S3790 S468F I503M G504A N505Y V508T I512T} ho-Δ | | |
| yHJC596 | MATα malt2Δ::MALT4^{S379Q S468F I503M G504A N505T V508T I512T} | yHJC207 | This study |
| | malt4Δ::MALT4^{S379Q S468F I503M G504A N505T V508T I512T} ho-Δ | | |
| yHJC597 | MATα malt2Δ::MALT4^{S379Q S468F I503M G504A N505K V508T I512T} | yHJC207 | This study |
| | malt4Δ::MALT4^{S379Q S468F I503M G504A N505K V508T I512T} ho-Δ | | |
| yHJC598 | MATα malt2Δ::MALT4^{S379Q S468F I503M G504A N505V V508T I512T} | yHJC207 | This study |
| | malt4Δ::MALT4^{S379Q S468F I503M G504A N505V V508T I512T} ho-Δ | | |
| yHJC599 | MATα malt2Δ::MALT4^{S379Q S468F I503M G504A N505S V508T I512T} | yHJC207 | This study |
| | malt4Δ::MALT4^{S379Q S468F I503M G504A N505S V508T I512T} ho-Δ | | |
| yHJC600 | MATα malt2Δ::MALT4^{S379Q S468F I503M G504A N505T V508T I512T} | yHJC207 | This study |
| | malt4Δ::MALT4^{S379Q S468F I503M G504A N505T V508T I512T} ho-Δ | | |
| yHJC602 | MATα malt2Δ::MALT4^{S379Q S468F I503M G504A N505T V508T I512T} | yHJC207 | This study |
| | malt4Δ::MALT4^{S379Q S468F I503M G504A N505T V508T I512T} ho-Δ | | |

TABLE 3
Oligonucleotides used in this study

| OLIGO | | SEQ ID | |
|---|---|---|---|
| ID | SEQUENCE | NO: | PURPOSE |
| OHJC1 | TGTATCGTAACTGCTGTCCTGACATTGTACCAACTGA | 21 | Mutagenesis of |
| | ACTCTGAGAAATGGAACTGGG | | MALT4 |
| OHJC2 | ACCCCAGTTCCATTTCTCAGAGTTCAGTTGGTACAAT | 22 | Mutagenesis of |
| | GTCAGGACAGCAGTTACGATAC | | MALT4 |
| OHJC3 | AATATCGTAGTCGCTGTTTTGATTTTGTACCAATTGAA | 23 | Mutagenesis of |
| | TTCAGAAAAATGGGATTGGG | | MALT4 |
| OHJC4 | ACCCCAATCCCATTTTTCTGAATTCAATTGGTACAAA | 24 | Mutagenesis of |
| | ATCAAAACAGCGACTACGAT | | MALT4 |
| OHJC5 | AATGCCCGTACAGGAGAATGGGAGATTTCGTCAGTTT | 25 | Construction of novel |
| | CTTGGCAGATTGGTTTATCT | | MALT3/MALT4 |
| | | | chimera |
| OHJC6 | TAAAGATAAACCAATCTGCCAAGAAACTGACGAAAT | 26 | Construction of novel |
| | CTCCCATTCTCCTGTACGGGCAT | | MALT3/MALT4 |
| | | | chimera |
| OHJC7 | GATGAAGTAACGCCAAATATTCTCGACGCTGCTATGC | 27 | Construction of novel |
| | AGGATGCGAAGGAGGCAGACGA | | MALT3/MALT4 |
| | | | chimera |
| OHJC8 | TTCGTCTGCCTCCTTCGCATCCTGCATAGCAGCGTCGA | 28 | Construction of novel |
| | GAATATTTGGCGTTACTTCATC | | MALT3/MALT4 |
| | | | chimera |
| OHJC9 | GATGAGGAAGTCCCAGATCTTCTCGATGAGGCGCAG | 29 | Construction of novel |
| | GATGCTAAGGAGGCCGACGATAG | | MALT3/MALT4 |
| | | | chimera |
| OHJC10 | ACTATCGTCGGCCTCCTTAGCATCCTGCGCCTCATCG | 30 | Construction of novel |
| | AGAAGATCTGGGACTTCCTCATC | | MALT3/MALT4 |
| | | | chimera |
| OHJC18 | AAAATGGGAAGTGGCGCTCTTCTAATGGTCGTTGCGT | 31 | Mutagenesis of |
| | TCTTTTACAACCTGGGGATTGCC | | MALT4 |
| OHJC19 | GCAAAAAACGACAGGGGCAATCCCCAGGTTGTAAAA | 32 | Mutagenesis of |
| | GAACGCAACGACCATTAGAAGAGC | | MALT4 |
| OHJC20 | AAAATGGGAAGTGGCGCTCTTCTAATGGTCGTTGCGT | 33 | Mutagenesis of |
| | TCTTGTACAACCTGGGGATTGCC | | MALT4 |
| OHJC21 | GCAAAAAACGACAGGGGCAATCCCCAGGTTGTACAA | 34 | Mutagenesis of |
| | GAACGCAACGACCATTAGAAGAGC | | MALT4 |
| OHJC24 | AGTCGCTGTTTTGATTTTGTACCAACTGAACTCTGAG | 35 | Mutagenesis of |
| | AAATGGGATTGGGGTGCCAAGTC | | MALT4 |
| OHJC25 | GCAGAATCCTCCCCAGAAAAAGCCTGACTTGGCACCC | 36 | Mutagenesis of |
| | CAATCCCATTTCTCAGAGTTCAG | | MALT4 |
| OHJC26 | AGTCGCTGTTTTGATTTTGTACCAACTGAACTCTGAG | 37 | Mutagenesis of |
| | AAATGGGACTGGGGTGCCAAGTC | | MALT4 |
| OHJC27 | GCAGAATCCTCCCCAGAAAAAGCCTGACTTGGCACCC | 38 | Mutagenesis of |
| | CAGTCCCATTTCTCAGAGTTCAG | | MALT4 |
| OHJC30 | CGATACTAACGCCGCCATCCAG | 39 | Screening for kanMX |
| | | | replacement at |
| | | | MALT2 |
| OHJC31 | CCATAATCTTGGCCGCACGC | 40 | Screening or |
| | | | sequencing of |
| | | | MALT2/MALT4 |
| OHJC32 | CGTCAATCGTATGTGAATGC | 41 | Screening for kanMX |
| | | | replacement at |
| | | | MALT4 |
| OHJC33 | TACAGATCTTACTGCGCAAC | 42 | Screening or |
| | | | sequencing of |
| | | | MALT2/MALT4 |
| OHJC34 | cgggtggcgaatgggactttTACAGGAGAATGGGAGATTTgttttaga | 43 | sgRNA targeting |
| | gctagaaatagc | | MALT2 and MALT4 |
| OHJC37 | cgggtggcgaatgggactttGTTACCCATCAAATCCACCGgttttaga | 44 | sgRNA targeting |
| | gctagaaatagc | | MALT2 and MALT4 |
| OHJC41 | ACCAAGACACTCAAAAAAAATTCCAAAAGCTATTAG | 45 | CDS insertion at |
| | GTAACTATGAAGGGTCTATCCTCA | | MALT2/MALT4 |
| OHJC42 | ACACGGAGAGTGATACCTTATCATCTGCTGCGCTAAG | 46 | CDS insertion at |
| | AGTCATCAATCCATTAGAGATGG | | MALT2/MALT4 |
| OHJC43 | ACCAAGACACTCAAAAAAAATTCCAAAAGCTATTAG | 47 | CDS insertion at |
| | GTAACTATGAAGGGCTTATCCTCA | | MALT2/MALT4 |
| OHJC44 | ACACGGAGAGTGATACCTTATCATCTGCTGCGCTAAG | 48 | CDS insertion at |
| | AGTCATCATAAATTCGTAATAGA | | MALT2/MALT4 |
| OHJC45 | ACCAAGACACTCAAAAAAAATTCCAAAAGCTATTAG | 49 | CDS insertion at |
| | GTAACTATGAAGAATATCATTTCG | | MALT2/MALT4 |
| OHJC49 | CATTTTCGACACTCCGAAGAAGG | 50 | Screening or |
| | | | sequencing of |
| | | | MALT2/MALT4 |
| OHJC50 | GGTATTTAATATATACACG | 51 | Screening or |
| | | | sequencing of |
| | | | MALT2/MALT4 |
| OHJC52 | CAAGGCCATGTCTTCTCGTT | 52 | Screening or |
| | | | sequencing of HO |
| OHJC53 | GTTTCTGGCCGAGCTACAAG | 53 | Screening or |
| | | | sequencing of HO |
| OHJC54 | CAAAACCCCTACAAAAACTGC | 54 | HO deletion |
| OHJC55 | CCACTAGCCTTTAAGCATGCGGGTACGGGTTTGTTGA | 55 | HO deletion |
| | AGT | | |
| OHJC56 | ACTTCAACAAACCCGTACCCGCATGCTTAAAGGCTAG | 56 | HO deletion |
| | TGG | | |
| OHJC57 | CTTGATGGTTTCTTCTCTCC | 57 | HO deletion |
| OHJC97 | ACACGGAGAGTGATACCTTATCATCTGCTGCGCTAAG | 58 | CDS insertion at |
| | AGTCATTATAATGCCTGCTGACT | | MALT2/MALT4 |
| OHJC121 | ATCCGTTCAAATTATAAAAGTCAC | 59 | MAT locus |
| | | | genotyping |
| OHJC122 | ACAGTTCATAAATAAACGTATGAGATC | 60 | MAT locus |
| | | | genotyping |
| OHJC123 | CGGAATATGGGACTACTTCG | 61 | MAT locus |
| | | | genotyping |
| OHJC137 | CTTGAACATGTGTGTTCTTTGTGG | 62 | Sequencing of |
| | | | seAGT1 |
| OHJC138 | TAACGATTGCCCATGAACTCGACC | 63 | Sequencing of |
| | | | seAGTI |
| OHJC139 | TCTGATTGCATTATCGTTGTCTCC | 64 | Sequencing of |
| | | | seAGTI |
| OHJC141 | CTCACGGTGCCAAAATGGGAAGTGGCGCTCTTCTAAT | 65 | Construction of novel |
| | GGTGGTCGCCTTCTTTTACAACC | | MALT3/MALT4 |
| | | | chimera |
| OHJC142 | AGACAACGGGAGCAATCCCCAGGTTGTAAAAGAAGG | 66 | Construction of novel |
| | CGACCACCATTAGAAGAGCGCCAC | | MALT3/MALT4 |
| | | | chimera |
| OHJC145 | AAATCAATTATTCTGGCTCGTAACGCCTATAATATGG | 67 | Mutagenesis of |
| | CAAATATCGTAGTCGCTGTTTTG | | MALT4 |
| OHJC146 | GCAAAAGACAACGGGAGCAATCCCCAGGTTGTAAAA | 68 | Mutagenesis of |
| | GAACGCAACGACCATTAGAAGAGC | | MALT4 |
| OHJC147 | CAAAATGGGAAGTGGCGCTCTTCTAATGGTCGTTGCG | 69 | Mutagenesis of |
| | TTCTTTTACAACCTGGGGATTGC | | MALT4 |
| OHJC148 | CAGTTGGTACAAAATCAAAACAGCGACTACGATATTT | 70 | Mutagenesis of |
| | GCCATATTATAGGCGTTACGAGC | | MALT4 |
| OHJC149 | GCTCGTAATGCGTACAACATGGCATGTATCGTAGTCG | 71 | Mutagenesis of |
| | CTGTTTTG | | MALT4 |
| OHJC150 | CAAAACAGCGACTACGATACATGCCATGTTGTACGCA | 72 | Mutagenesis of |
| | TTACGAGC | | MALT4 |
| OHJC151 | GCGTACAACATAGGATGTATCGTAACTGCTGTCCTGA | 73 | Mutagenesis of |
| | CATTGTACCAACTGAACTCTGAG | | MALT4 |
| OHJC152 | TCAGTTGGTACAATGTCAGGACAGCAGTTACGATACA | 74 | Mutagenesis of |
| | TCCTATGTTGTACGCATTACGAG | | MALT4 |
| OHJC153 | GTCGTTGTTGGTTTCCACAACG | 75 | Sequencing of |
| | | | MALT4 |
| OHJC154 | CGTTGTGGAAACCAACAACGAC | 76 | Sequencing of |
| | | | MALT4 |
| OHJC155 | GAAGCTGTCAGACGATGAAGGC | 77 | Sequencing of |
| | | | MALT4 |
| OHJC162 | GACTTCGACGATCTTAGTAGCAGCG | 78 | Screening or |
| | | | sequencing of |
| | | | MALT4 |
| OHJC165 | GCAGCACGTCTGGAGCGTACAGATCTTACTGCGCAAC | 79 | CDS insertion at |
| | ATATAAAGAGGCGTGATATGCTC | | MALT2/MALT4 |
| OHJC166 | TTACATAAAACCAACGAAATACCTTAACCAAGTAATA | 80 | CDS insertion at |
| | AATAATAAAGCTAATGTGCTAGG | | MALT2/MALT4 |
| OHJC167 | TAATACGTACTCAGAGACACGC | 81 | Screening or |
| | | | sequencing of |
| | | | MALT2/MALT4 |
| OHJC168 | ACTTTACGGAGGAATAAAGAGCC | 82 | Screening or |
| | | | sequencing of |
| | | | MALT2/MALT4 |
| OHJC200 | AATGGGAAGTGGCGCTCTTCTAATGGTGGTCGCCTTC | 83 | Mutagenesis of |
| | TCATACAACCTGGGGATTGCTCC | | MALT4 |
| OHJC201 | GCAAAAGACAACGGGAGCAATCCCCAGGTTGTATGA | 84 | Mutagenesis of |
| | GAAGGCGACCACCATTAGAAGAGC | | MALT4 |
| OHJC202 | CTAAATCAATTATTCTGGCTCGTAACGCCTATAATAT | 85 | Mutagenesis of |
| | AGCATGTATCGTAACTGCTGTCC | | MALT4 |
| OHJC203 | TTGGTACAATGTCAGGACAGCAGTTACGATACATGCT | 86 | Mutagenesis of |
| | ATATTATAGGCGTTACGAGCCAG | | MALT4 |
| OHJC204 | AAATCAATTATTCTGGCTCGTAACGCCTATAATATGG | 87 | Mutagenesis of |
| | GATGTATCGTAACTGCTGTCCTG | | MALT4 |
| OHJC205 | TTGGTACAATGTCAGGACAGCAGTTACGATACATCCC | 88 | Mutagenesis of |
| | ATATTATAGGCGTTACGAGCCAG | | MALT4 |
| OHJC206 | ATCAATTATTCTGGCTCGTAACGCCTATAATATGGCA | 89 | Mutagenesis of |
| | AATATCGTAACTGCTGTCCTGAC | | MALT4 |
| OHJC207 | CAGTTGGTACAATGTCAGGACAGCAGTTACGATATTT | 90 | Mutagenesis of |
| | GCCATATTATAGGCGTTACGAGC | | MALT4 |
| OHJC208 | GCTCGTAACGCCTATAATATGGCATGTATCGTAGTCG | 91 | Mutagenesis of |
| | CTGTCCTGACATTGTACCAACTG | | MALT4 |
| OHJC209 | TCAGAGTTCAGTTGGTACAATGTCAGGACAGCGACTA | 92 | Mutagenesis of |
| | CGATACATGCCATATTATAGGCG | | MALT4 |
| OHJC210 | GCCTATAATATGGCATGTATCGTAACTGCTGTCCTGA | 93 | Mutagenesis of |
| | TTTTGTACCAACTGAACTCTGAG | | MALT4 |
| OHJC211 | CCAGTTCCATTTCTCAGAGTTCAGTTGGTACAAAATC | 94 | Mutagenesis of |
| | AGGACAGCAGTTACGATACATGC | | MALT4 |
| OHJC290 | GCTAAAAAGAACAACGCCACAATCAATGTGTAACGA | 95 | Construction of novel |
| | TTACCCATCAAATCCACCGAGGGA | | MALT3/MALT4 |
| | | | chimera |
| OHJC291 | GGTTTACAGTTGACAGGTCCCTCGGTGGATTTGATGG | 96 | Construction of novel |
| | GTAATCGTTACACATTGATTGTG | | MALT3/MALT4 |
| | | | chimera |
| OHJC292 | ACAGCCCAATCTGCCAAGATACCGAAATCTCCCATTC | 97 | Construction of novel |
| | TCCTGTCTTGCTATTCAGAGAAC | | MALT3/MALT4 |
| | | | chimera |
| OHJC293 | CAGAAGGAATATGGTTCTCTGAATAGCAAGACAGGA | 98 | Construction of novel |
| | GAATGGGAGATTTCGGTATCTTGG | | MALT3/MALT4 |
| | | | chimera |
| OHJC294 | AATAAGTTGTCAGGTAGTATCTTAAAGCCAAAGGACA | 99 | Construction of novel |
| | GATTTCAGAGGCATAAGACACAG | | MALT3/MALT4 |
| | | | chimera |
| OHJC295 | TTCCAATGTCTGACTGTGTCTTATGCCTCTGAAATCTG | 100 | Construction of novel |
| | TCCTTTGGCTTTAAGATACTAC | | MALT3/MALT4 |
| | | | chimera |
| OHJC296 | GAATAATACTAAAAGTGAATGCTGTTTCAGTGCTAAC | 101 | Construction of novel |
| | ACCGGCTTTCTCGTAGAAGTAAG | | MALT3/MALT4 |
| | | | chimera |
| OHJC297 | CTAATAGGTTACTCAACTTACTTCTACGAGAAAGCCG | 102 | Construction of novel |
| | GTGTTAGCACTGAAACAGCATTC | | MALT3/MALT4 |
| | | | chimera |
| OHJC298 | TAGTCTTCCTTTCTTGACTAACCACCATGGGGATTCAG | 103 | Construction of novel |
| | GTGCAAAGAATATCCCTATTGC | | MALT3/MALT4 |
| | | | chimera |
| OHJC299 | TGGCCTCTTCCCCTGGCAATAGGGATATTCTTTGCACC | 104 | Construction of novel |
| | TGAATCCCCATGGTGGTTAGTC | | MALT3/MALT4 |
| | | | chimera |
| OHJC300 | CGGAGAGTGATACCTTATCATCTGCTGCGCTAAGAGT | 105 | CDS insertion at |
| | CATCATAAATTCGTAATAGATGG | | MALT2/MALT4 |
| OHJC301 | ATGTCAAAACAGCAGTTACGATACATCCCATGTTGTA | 106 | Mutagenesis of |
| | CGCATTACGAGCTAAAATGATCG | | MALT4 |
| OHJC302 | GCGTACAACATGGGATGTATCGTAACTGCTGTTTTGA | 107 | Mutagenesis of |
| | CATTGTACCAACTGAACTCTGAG | | MALT4 |
| OHJC303 | ATGTCAAAACAGCGACTACGATACATCCCATGTTGTA | 108 | Mutagenesis of |
| | CGCATTACGAGCTAAAATGATCG | | MALT4 |
| OHJC304 | GCGTACAACATGGGATGTATCGTAGTCGCTGTTTTGA | 109 | Mutagenesis of |
| | CATTGTACCAACTGAACTCTGAG | | MALT4 |
| OHJC326 | ATAAACAAGCTATTCTTGTTCTTCTCCGGTTGATACAG | 110 | Construction of novel |
| | TCCCTCAGACAATCCAAATAGG | | MALT3/MALT4 |
| | | | chimera |
| OHJC327 | AAGCTGTCAGACGATGAAGGCTCCTATTTGGATTGTC | 111 | Construction of novel |
| | TGAGGGACTGTATCAACCGGAGA | | MALT3/MALT4 |
| | | | chimera |
| OHJC328 | CTGTTCCATTCTCCCTTTCTTGATCAACCACCATGGAG | 112 | Construction of novel |
| | ATTCAGGTGCAAAGAATATCGC | | MALT3/MALT4 |
| | | | chimera |
| OHJC329 | ATCTGGCCTGTTCCTCTAGCAATAGCGATATTCTTTGC | 113 | Construction of novel |
| | ACCTGAATCTCCATGGTGGTTG | | MALT3/MALT4 |
| | | | chimera |
| OHJC330 | GCAAATGGTAGCTTATATCCCAAATCCGAGTCTGGGT | 114 | Construction of novel |
| | ATTTGTTTTGGGAATTTTTCATG | | MALT3/MALT4 |
| | | | chimera |
| OHJC331 | CAGCTTTTTGCTGCAGGCATCATGAAAAATTCCCAAA | 115 | Construction of novel |
| | ACAAATACCCAGACTCGGATTTG | | MALT3/MALT4 |
| | | | chimera |
| OHJC332 | GATTCGAATATGTCGTCAGGTAGTACCTCAGCGCCAT | 116 | Construction of novel |
| | AGGACAGATTTCGGAAGCATATG | | MALT3/MALT4 |
| | | | chimera |
| OHJC333 | GTTTCCAATGTTTGACAGTCTCATATGCTTCCGAAATC | 117 | Construction of novel |
| | TGTCCTATGGCGCTGAGGTACT | | MALT3/MALT4 |
| | | | chimera |
| OHJC334 | CATTCTCCCTTTCTTGATCAACCACCATGGAGATTCAG | 118 | Construction of novel |
| | GTGCAAAGAATATCGCTATTGC | | MALT3/MALT4 |
| | | | chimera |
| OHJC335 | CCATGTAATCTACAAAAGGACC | 119 | Sequencing of |
| | | | MALT3 |
| OHJC336 | GACCAATCCAACATAAACAAGC | 120 | Sequencing of |
| | | | MALT3 |
| OHJC378 | CCAAATTAAAACAGCCAGGCA | 121 | Sequencing of |
| | | | MALT3 |
| OHJC379 | GACTCGGACTTAGGATATACGC | 122 | Sequencing of |
| | | | MALT3 |
| OHJC380 | GACCGTTATACTGTTCATTATAGGC | 123 | Sequencing of |
| | | | MALT3 |
| OHJC381 | ATACCGAAATCTCCCATTCTCC | 124 | Sequencing of |
| | | | MALT3 |
| OHJC382 | TTTGCCTGCTCTAGTCTTCC | 125 | Sequencing of |
| | | | MALT3 |
| OHJC383 | GAAAACGTATCCCAAAGCTGC | 126 | Sequencing of |
| | | | MALT3 |
| OHJC384 | CCTTTGGCTTTAAGATACTACCTG | 127 | Sequencing of |
| | | | MALT3 |
| OHJC385 | GTCGCCTTCTTTTACAACCTG | 128 | Sequencing of |
| | | | MALT3 |
| OHJC386 | GAAGAAGCCTGACTTTGCAC | 129 | Sequencing of |
| | | | MALT3 |
| OHJC387 | GGTAGCTTATATCCCAAATCCG | 130 | Sequencing of |
| | | | MALT3 |
| OHJC388 | AGAACTGCTGGTGAGTATGG | 131 | Sequencing of |
| | | | MALT3 |
| OHJC399 | CGGCTAAAAAGAACAACGCCACAATCAATGTGTAAC | 132 | Construction of novel |
| | GATTACCCATCAAATCCACCGAGG | | MALT3/MALT4 |
| | | | chimera |
| OHJC400 | GTTTACAGTTGACAGGTCCCTCGGTGGATTTGATGGG | 133 | Construction of novel |
| | TAATCGTTACACATTGATTGTGG | | MALT3/MALT4 |
| | | | chimera |
| OHJC401 | TAGTCTTCCTTTCTTGACTAACCACCATGGGGATTCAG | 134 | Construction of novel |
| | GTGCAAAGAATATCCCTATTGC | | MALT3/MALT4 |
| | | | chimera |
| OHJC402 | GATTTGGCCTCTTCCCCTGGCAATAGGGATATTCTTTG | 135 | Construction of novel |
| | CACCTGAATCCCCATGGTGGTT | | MALT3/MALT4 |
| | | | chimera |
| OHJC403 | TTTGACCGGCCCAGCACAGACAGGCTATTCTCGTTCT | 136 | Construction of novel |
| | CCTCCGGTTGATACAGTCCTTCA | | MALT3/MALT4 |
| | | | chimera |
| OHJC404 | GGTCATACTGGGATTGTGTGAAGGACTGTATCAACCG | 137 | Construction of novel |
| | GAGGAGAACGAGAATAGCCTGTC | | MALT3/MALT4 |
| | | | chimera |
| OHJC405 | GAATAATACTAAAAGTGAATGCTGTTTCAGTGCTAAC | 138 | Construction of novel |
| | ACCGGCTTTCTCGTAGAAGTAAG | | MALT3/MALT4 |
| | | | chimera |
| OHJC406 | ACTAATAGGTTACTCAACTTACTTCTACGAGAAAGCC | 139 | Construction of novel |
| | GGTGTTAGCACTGAAACAGCATT | | MALT3/MALT4 |
| | | | chimera |
| OHJC407 | TGTCTGTGCTGGGCCGGTCAAACTGTTTGTGGTACAC | 140 | Mutagenesis of |
| | AACTAATAGGTTACTCAACTTAC | | MALT4 |
| OHJC408 | GCTTTCTCGTAGAAGTAAGTTGAGTAACCTATTAGTT | 141 | Mutagenesis of |
| | GTGTACCACAAACAGTTTGACCG | | MALT4 |
| OHJC409 | TTGTTTATGTTGGATTGGTCAAACCACTTGTGGTGCGT | 142 | Mutagenesis of |
| | CACTGATAGGATATTCAACCTA | | MALT4 |
| OHJC410 | GCCTTTTCGTAAAAATAGGTTGAATATCCTATCAGTG | 143 | Mutagenesis of |
| | ACGCACCACAAGTGGTTTGACCA | | MALT4 |
| OHJC411 | TTTACAAATGACTGGTCCTTTTGTAGATTACATGGGT | 144 | Construction of novel |
| | AACCGCTACACATTGATTATGGC | | MALT3/MALT4 |
| | | | chimera |
| OHJC412 | AGTTAAGAACATCAACGCCATAATCAATGTGTAGCGG | 145 | Construction of novel |
| | TTACCCATGTAATCTACAAAAGG | | MALT3/MALT4 |
| | | | chimera |
| OHJC413 | TGTTTCCAATGTTTGACAGTCTCATATGCTTCCGAAAT | 146 | Construction of novel |
| | CTGTCCTATGGCGCTGAGGTAC | | MALT3/MALT4 |
| | | | chimera |
| OHJC414 | CGAATATGTCGTCAGGTAGTACCTCAGCGCCATAGGA | 147 | Construction of novel |
| | CAGATTTCGGAAGCATATGAGAC | | MALT3/MALT4 |
| | | | chimera |
| OHJC415 | TGTTTCCAATGTCTGACTGTGTCTTATGCCTCTGAAAT | 148 | Construction of novel |
| | CTGTCCTTTGGCTTTAAGATAC | | MALT3/MALT4 |
| | | | chimera |
| OHJC416 | AATAAGTTGTCAGGTAGTATCTTAAAGCCAAAGGACA | 149 | Construction of novel |
| | GATTTCAGAGGCATAAGACACAG | | MALT3/MALT4 |
| | | | chimera |
| OHJC427 | AGCCTGTCTGTGCTGGGCCGGTCAAACTGTTTGTGGT | 150 | Mutagenesis of |
| | ACATCACTAATAGGTTACTCAAC | | MALT4 |
| OHJC428 | CTTTCTCGTAGAAGTAAGTTGAGTAACCTATTAGTGA | 151 | Mutagenesis of |
| | TGTACCACAAACAGTTTGACCGG | | MALT4 |
| OHJC429 | TGTCTGTGCTGGGCCGGTCAAACTGTTTGTGGTGCGC | 152 | Mutagenesis of |
| | AACTAATAGGTTACTCAACTTAC | | MALT4 |
| OHJC430 | ACCGGCTTTCTCGTAGAAGTAAGTTGAGTAACCTATT | 153 | Mutagenesis of |
| | AGTTGCGCACCACAAACAGTTTG | | MALT4 |
| OHJC431 | CTGTGCTGGGCCGGTCAAACTGTTTGTGGTACATCAC | 154 | Mutagenesis of |
| | TAATAGGTTACTCAACTTACTTC | | MALT4 |
| OHJC432 | CTTTCTCGTAGAAGTAAGTTGAGTAACCTATTAGTGA | 155 | Mutagenesis of |
| | TGTACCACAAACAGTTTGACCGG | | MALT4 |
| OHJC433 | GCTGGGCCGGTCAAACTGTTTGTGGTGCGCAACTAAT | 156 | Mutagenesis of |
| | AGGTTACTCAACTTACTTCTACG | | MALT4 |
| OHJC434 | GGCTTTCTCGTAGAAGTAAGTTGAGTAACCTATTAGT | 157 | Mutagenesis of |
| | TGCGCACCACAAACAGTTTGACC | | MALT4 |
| OHJC435 | CCTTCACTCATCTCTCCATACACG | 158 | Screening or |
| | | | sequencing of |
| | | | MALT2/MALT4 |
| OHJC436 | CTGAATGATCTGGTGAACTTTACGG | 159 | Screening or |
| | | | sequencing of |
| | | | MALT2/MALT4 |
| OHJC437 | ATCAATTATTCTGGCTCGTAACGCCTATAATATGGCA | 160 | Mutagenesis of |
| | CAAATCGTAACTGCTGTCCTGAC | | MALT4 |
| OHJC438 | TCAGTTGGTACAATGTCAGGACAGCAGTTACGATTTG | 161 | Mutagenesis of |
| | TGCCATATTATAGGCGTTACGAG | | MALT4 |
| OHJC439 | ATCAATTATTCTGGCTCGTAACGCCTATAATATGGCA | 162 | Mutagenesis of |
| | GATATCGTAACTGCTGTCCTGAC | | MALT4 |
| OHJC440 | TCAGTTGGTACAATGTCAGGACAGCAGTTACGATATC | 163 | Mutagenesis of |
| | TGCCATATTATAGGCGTTACGAG | | MALT4 |
| OHJC441 | GCCTATAATATGGCATGTATCGTAACTGCTGTCCTGG | 164 | Mutagenesis of |
| | TTTTGTACCAACTGAACTCTGAG | | MALT4 |
| OHJC442 | CCCAGTTCCATTTCTCAGAGTTCAGTTGGTACAAAAC | 165 | Mutagenesis of |
| | CAGGACAGCAGTTACGATACATG | | MALT4 |
| OHJC443 | GCTGGGCCGGTCAAACTGTTTGTGGTGCGTGTCTAAT | 166 | Mutagenesis of |
| | AGGTTACTCAACTTACTTCTACG | | MALT4 |
| OHJC444 | GGCTTTCTCGTAGAAGTAAGTTGAGTAACCTATTAGA | 167 | Mutagenesis of |
| | CACGCACCACAAACAGTTTGACC | | MALT4 |
| OHJC445 | GCTGGGCCGGTCAAACTGTTTGTGGTGCGGAACTAAT | 168 | Mutagenesis of |
| | AGGTTACTCAACTTACTTCTACG | | MALT4 |
| OHJC446 | GGCTTTCTCGTAGAAGTAAGTTGAGTAACCTATTAGT | 169 | Mutagenesis of |
| | TCCGCACCACAAACAGTTTGACC | | MALT4 |
| OHJC447 | ATCAATTATTCTGGCTCGTAACGCCTATAATATGGCAt | 170 | Mutagenesis of |
| | ttATCGTAACTGCTGTCCTGAC | | MALT4 |
| OHJC448 | ATCAATTATTCTGGCTCGTAACGCCTATAATATGGCAt | 171 | Mutagenesis of |
| | tgATCGTAACTGCTGTCCTGAC | | MALT4 |
| OHJC449 | ATCAATTATTCTGGCTCGTAACGCCTATAATATGGCAt | 172 | Mutagenesis of |
| | atATCGTAACTGCTGTCCTGAC | | MALT4 |
| OHJC450 | ATCAATTATTCTGGCTCGTAACGCCTATAATATGGCAC | 173 | Mutagenesis of |
| | atATCGTAACTGCTGTCCTGAC | | MALT4 |
| OHJC451 | ATCAATTATTCTGGCTCGTAACGCCTATAATATGGCAa | 174 | Mutagenesis of |
| | ttATCGTAACTGCTGTCCTGAC | | MALT4 |
| OHJC452 | ATCAATTATTCTGGCTCGTAACGCCTATAATATGGCAa | 175 | Mutagenesis of |
| | tgATCGTAACTGCTGTCCTGAC | | MALT4 |
| OHJC453 | ATCAATTATTCTGGCTCGTAACGCCTATAATATGGCAa | 176 | Mutagenesis of |
| | aaATCGTAACTGCTGTCCTGAC | | MALT4 |
| OHJC454 | ATCAATTATTCTGGCTCGTAACGCCTATAATATGGCAg | 177 | Mutagenesis of |
| | ttATCGTAACTGCTGTCCTGAC | | MALT4 |
| OHJC455 | ATCAATTATTCTGGCTCGTAACGCCTATAATATGGCAg | 178 | Mutagenesis of |
| | aaATCGTAACTGCTGTCCTGAC | | MALT4 |
| OHJC456 | ATCAATTATTCTGGCTCGTAACGCCTATAATATGGCAt | 179 | Mutagenesis of |
| | ctATCGTAACTGCTGTCCTGAC | | MALT4 |
| OHJC457 | ATCAATTATTCTGGCTCGTAACGCCTATAATATGGCAt | 180 | Mutagenesis of |
| | ggATCGTAACTGCTGTCCTGAC | | MALT4 |
| OHJC458 | ATCAATTATTCTGGCTCGTAACGCCTATAATATGGCAC | 181 | Mutagenesis of |
| | caATCGTAACTGCTGTCCTGAC | | MALT4 |
| OHJC459 | ATCAATTATTCTGGCTCGTAACGCCTATAATATGGCAa | 182 | Mutagenesis of |
| | gaATCGTAACTGCTGTCCTGAC | | MALT4 |
| OHJC460 | ATCAATTATTCTGGCTCGTAACGCCTATAATATGGCAa | 183 | Mutagenesis of |
| | ctATCGTAACTGCTGTCCTGAC | | MALT4 |
| OHJC461 | ATCAATTATTCTGGCTCGTAACGCCTATAATATGGCAg | 184 | Mutagenesis of |
| | ctATCGTAACTGCTGTCCTGAC | | MALT4 |
| OHJC462 | ATCAATTATTCTGGCTCGTAACGCCTATAATATGGCAg | 185 | Mutagenesis of |
| | gtATCGTAACTGCTGTCCTGAC | | MALT4 |
| OHJC463 | CATATTATAGGCGTTACGAGCCAG | 186 | Mutagenesis of |
| | | | MALT4 |
| OHJC464 | TGCCATATTATAGGCGTTACGAG | 187 | Mutagenesis of |
| | | | MALT4 |
| OHRW347 | AGCAATCCCCAGGTTGTAAAAGAAGGCGACCACCAT | 188 | Construction of novel |
| | TAGAAGAGCGCCACTTCCCAT | | MALT3/MALT4 |
| | | | chimera |
| OHRW348 | ATGGGAAGTGGCGCTCTTCTAATGGTGGTCGCCTTCT | 189 | Construction of novel |
| | TTTACAACCTGGGGATTGCT | | MALT3/MALT4 |
| | | | chimera |
| OHRW349 | GGGACTATGTTTTGCGACGTTAGTATGGGCTGTCTTT | 190 | Construction of novel |
| | GATCTGCCCGAAACCGCA | | MALT3/MALT4 |
| | | | chimera |
| OHRW350 | TGCGGTTTCGGGCAGATCAAAGACAGCCCATACTAAC | 191 | Construction of novel |
| | GTCGCAAAACATAGTCCC | | MALT3/MALT4 |
| | | | chimera |

Transformation of S. eubayanus was performed by the PEG/LiAc/carrier DNA method (Gietz and Schiestl 2007) with minor modifications (Baker and Hittinger 2019). CRISPR-mediated gene deletions and insertions were achieved by co-transformation of pXIPHOS vectors (Kuang et al. 2018) and repair templates for homologous recombination. Repair templates were purified PCR products consisting of single linear fragments, multiple linear fragments for in vivo assembly, or recombinant amplicons generated by overlap extension PCR, depending on the application. All repair templates were amplified using Phusion polymerase (New England Biolabs) per the manufacturer's instructions and purified using QiaQuick or MinElute spin columns (Qiagen).

We assessed transporter function via expression from the native MALT4 locus in yHJC207, a haploid derivative of the wild strain yHKS210 that was constructed as previously described (Crandall et al. 2023). Because the MALT2 and MALT4 loci are recent duplicates and almost identical at the nucleotide level, transporter variants were inserted into both loci out of necessity. Both MALT2 and MALT4 were simultaneously deleted using CRISPR-Cas9 and replaced with kanMX. Novel transporter variants, as well as MALT434 and S. eubayanus AGT1 positive controls, were subsequently inserted into both loci by co-transformation with a pXIPHOS vector expressing Cas9 and a gRNA targeting kanMX (Lee et al. 2021). Transformants were cured of plasmids, and the inserted alleles were sequenced.

Quantitative Growth Measurements of S. eubayanus Strains

Strains were streaked to single colonies on YPD plates, arrayed in 96-well plates in a randomized layout, and precultured in YPD at room temperature for 72 hours with gentle shaking. Precultures were serially diluted in minimal medium (0.5% ammonium sulfate, 0.017% Yeast Nitrogen Base) and inoculated into minimal medium containing 2% sugars in 96-well plates at a final dilution of 10⁻⁴. OD₆₀₀ was measured every hour for 7 days using a SPECTROstar Omega plate reader (BMG Labtech) equipped with a microplate stacker. Raw growth data was summarized using GCAT (Bukhman et al. 2015). Area under the curve (AUC) measurements for growth on maltotriose, normalized to a common negative control within each experiment, were used as a response variable in linear models with protein identity (MalT3 or MalT4) at each domain or at key amino acid sites as categorical predictor variables. The effects of protein identity at some single regions and for many pairwise interactions could not be estimated due to singularities. We tested for evidence of epistasis by statistically comparing additive models and those with interaction terms (Li and Fay 2019). The amino acid properties compiled to test associations with transporter function included chemical composition, polarity, and volume (Grantham 1974), aromaticity (Xia and Li 1998), hydropathy (JANIN 1979; Kyte and Doolittle 1982; Hopp and Woods 1983; Eisenberg et al. 1984; Rose et al. 1985; Cornette et al. 1987; Engelman et al. 2003), and BLOSUM similarity (Henikoff and Henikoff 1992). Some matrices were compiled from Braun (Braun 2018). For dimensionality reduction, BLOSUM similarity was omitted.

### Quantitative Growth Measurements of Saccharomycotina Yeasts

Growth on α-glucosides was measured for the strains whose genome annotations were analyzed, which were primarily the type strains for their respective species. Cryopreserved strains were inoculated directly to YPD in 96-well plates and incubated for 7 days at room temperature. Some slow-growing species failed to revive during this time frame and were removed from further analysis, and we did not phenotype opportunistic pathogens, ultimately resulting in data for 287 species. Precultures were inoculated to minimal medium with 1% sugar or no added carbon source using a pinning tool, incubated for 7 days at room temperature, and re-inoculated to new plates containing the same medium. OD₆₀₀ of the second round of growth was measured every hour using a SPECTROstar Omega plate reader (BMG Labtech) equipped with a microplate stacker. The growth experiments were performed four times independently. Raw growth data was summarized using Growthcurver (Sprouffske and Wagner 2016). Wells with poor model fits were discarded, and each curve was manually inspected to identify species with unreliable growth curves (Opulente et al. 2024). Growth on each carbon source was normalized to the average growth of the same species in medium with no added carbon to control for background growth. Caper (cran.r-project.org/web/packages/caper/index.html) was used to fit phylogenetically corrected regressions (PGLSs) to growth data and square-root transformed Agt number, using the rooted ML species phylogeny (X. X. Shen et al. 2018).

### Structure Prediction and Analyses

Structural models for MalT434 were generated using four different software: AlphaFold2 (Jumper et al. 2021), Phyre2 (Kelley et al. 2015), I-TASSER (Yang et al. 2015), and SWISS-MODEL (A. Waterhouse et al. 2018). All gave extremely similar results across the structured region (mean and SD pairwise RMSD: 1.61±0.51 Å), and AlphaFold2 models for all proteins of interest were generated and used for further analysis. Docking of maltotriose was performed using SwissDock (Grosdidier et al. 2011). Structure models and docking results were visualized in PyMol v2.5 (Schrödinger, LLC).

### Genome Annotation

To improve the quality of existing gene models, publicly available genome assemblies of 332 Saccharomycotina yeast species (X.-X. Shen et al. 2018) were re-annotated de novo. For consistency, we retained the assembly and species names, although some species have since been renamed; consult MycoBank (www.mycobank.org) for the most up-to-date taxonomic information. Repetitive sequences were softmasked with RepeatMasker v4.1.2, and protein-coding genes were annotated using ab inito predictors AUGUSTUS v3.4.0 (Stanke et al. 2008) and GeneMark-EP+ v4.6.1 (Brůna et al. 2020) in BRAKER (Brůna et al. 2021), with Saccharomyceses proteins in OrthoDB v10 (Kriventseva et al. 2019) as homology evidence and using the --fungus mode. Where applicable, the longest transcript of each gene was retained. BUSCO v5.7.0 (Manni et al. 2021) was used to assess the completeness of the new and preexisting genome annotations using single-copy yeast orthologs in OrthoDB v10 (R. M. Waterhouse et al. 2018).

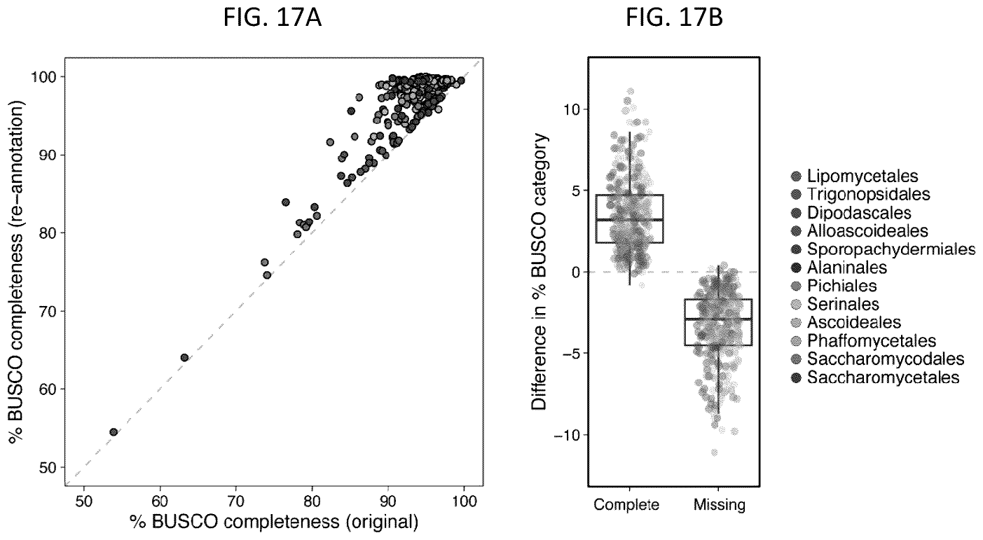

This approach was chosen so as to generate a useful community resource in two ways: first, to enable direct comparisons with a larger, partially overlapping dataset of yeast genomes published recently (Opulente et al. 2024), which were annotated using identical methods; and second, to facilitate future studies by significantly improving the quality of annotations for the widely used 332-genomes dataset. Median annotation completeness was increased from 94.6% to 98.8%, while the median percentage of missing BUSCO genes decreased to 0.9% from 4.6% (both p<2.2×10⁻¹⁶, two-sided 1-tests; FIG. 17).

### Phylogenetic Analyses

The amino acid translations of the newly predicted protein-coding genes were queried by BLASTp+ v2.9 (Camacho et al. 2009) using characterized Saccharomyces cerevisiae sugar transporters (Mal31, Agt1, Gal2, Hxt1-5, Hxt7) retrieved from SGD (Wong et al. 2023). BLAST subjects less than 400 or greater than 1000 amino acids in length were discarded to remove partial or fused annotations, based on distributions of sugar porter length in TCDB (Saier et al. 2006; Saier et al. 2021). Remaining proteins were annotated with their most similar S. cerevisiae homolog using a reciprocal BLASTp search against all translated ORFs in S. cerevisiae, which were retrieved from SGD. Protein sequences were aligned using the E-INS-i strategy of MAFFT v7.222 (Katoh et al. 2002; Katoh et al. 2005; Katoh and Standley 2013), and the alignment was trimmed with trimAL v1.4.22 (Capella-Gutiérrez et al. 2009) using the --gappyout parameter. The phylogeny was inferred using IQ-TREE v2.2.2.7 (Minh et al. 2020) with 1000 bootstraps (Hoang et al. 2018) and automatic substitution model selection (Kalyaanamoorthy et al. 2017). Due to the high conservation of MFS proteins, this dataset contained a small proportion of non-sugar porter MFS proteins, primarily belonging to the drug:proton antiporter family. These were retained in the alignment and tree inference to test the assumption of sugar porter monophyly. As expected, the sugar porters and non-sugar porter MFS proteins formed well-supported reciprocally monophyletic clades. The α-glucoside transporter phylogeny was refined by re-aligning the proteins from that clade and inferring the phylogeny as before, albeit with 10 independent runs of IQ-TREE with 10000 bootstrap replicates each and secondary branch support assessment by SH-aLRT tests. Trees were visualized and annotated in iTOL (Letunic and Bork 2021).

### REFERENCES

Abramson J, Smirnova I, Kasho V, Verner G, Kaback H R, Iwata S. 2003. Structure and mechanism of the lactose permease of Escherichia coli. Science (1979) [Internet] 301:610-615.
Alves S L, Herberts R A, Hollatz C, Trichez D, Miletti L C, de Araujo P S, Stambuk B U. 2008. Molecular Analysis of Maltotriose Active Transport and Fermentation by Saccharomyces cerevisiae Reveals a Determinant Role for the AGT1 Permease. Appl Environ Microbiol [Internet] 74:1494-1501.
Arguello J R, Chen Y, Yang S, Wang W, Long M. 2006. Origination of an X-Linked Testes Chimeric Gene by Illegitimate Recombination in Drosophila. PLOS Genet [Internet] 2:e77.
Baker E P, Hittinger C T. 2019. Evolution of a novel chimeric maltotriose transporter in Saccharomyces eubayanus from parent proteins unable to perform this function. Zhang J, editor. PLOS Genet [Internet] 15:e1007786.
Baker E P, Sayegh R, Kohler K M, Borman W, Goodfellow C K, Brush E R, Barber M F. 2022. Evolution of host-microbe cell adherence by receptor domain shuffling. Elife [Internet] 11.
Bank C, Hietpas R T, Jensen J D, Bolon DNA. 2015. A Systematic Survey of an Intragenic Epistatic Landscape. Mol Biol Evol [Internet] 32:229-238.
Ben-David M, Soskine M, Dubovetskyi A, Cherukuri K-P, Dym O, Sussman J L, Liao Q, Szeler K, Kamerlin S C L, Tawfik D S. 2020. Enzyme Evolution: An Epistatic Ratchet versus a Smooth Reversible Transition. Barlow M, editor. Mol Biol Evol [Internet] 37:1133-1147.
Bergin S A, Allen S, Hession C, Cinnéide EÓ, Ryan A, Byrne K P, Cróinín TÓ, Wolfe K H, Butler G, Morrissey J. 2022. Identification of European isolates of the lager yeast parent Saccharomyces eubayanus. FEMS Yeast Res [Internet] 22:1-9.
Bittihn P, Tsimring L S. 2017. Gene Conversion Facilitates Adaptive Evolution on Rugged Fitness Landscapes. Genetics [Internet] 207:1577-1589.
Bosshart P D, Fotiadis D. 2019. Secondary Active Transporters. In: Subcellular Biochemistry. Vol. 92. p. 275-299.
Braun E L. 2018. An evolutionary model motivated by physicochemical properties of amino acids reveals variation among proteins. Bioinformatics 34:1350-i356.
Brickwedde A, Brouwers N, Brock M van den, Gallego Murillo J S, Fraiture J L, Pronk J T, Daran J M G. 2018. Structural, physiological and regulatory analysis of maltose transporter genes in Saccharomyces eubayanus CBS 12357T. Front Microbiol 9:1-18.
Bridgham J T, Carroll S M, Thornton J W. 2006. Evolution of hormone-receptor complexity by molecular exploitation. Science (1979) [Internet] 312:97-101.
Bridgham J T, Ortlund E A, Thornton J W. 2009. An epistatic ratchet constrains the direction of glucocorticoid receptor evolution. Nature [Internet] 461:515-519.
Brouwers N, Brickwedde A, Gorter de Vries A R, van den Broek M, Weening S M, van den Eijnden L, Diderich J A, Bai F Y, Pronk J T, Daran J M G. 2019. Himalayan saccharomyces eubayanus genome sequences reveal genetic markers explaining heterotic maltotriose consumption by saccharomyces pastorianus hybrids. Appl Environ Microbiol [Internet] 85.
Brouwers N, Gorter de Vries A R, van den Brock M, Weening S M, Elink Schuurman T D, Kuijpers N G A, Pronk J T, Daran J-MG. 2019. In vivo recombination of Saccharomyces cubayanus maltose-transporter genes yields a chimeric transporter that enables maltotriose fermentation. Zhang J, editor. PLOS Genet [Internet] 15:01007853.
Brown C A, Murray A W, Verstrepen K J. 2010. Rapid Expansion and Functional Divergence of Subtelomeric Gene Families in Yeasts. Current Biology [Internet] 20:895-903.
Brůna T, Hoff K J, Lomsadze A, Stanke M, Borodovsky M. 2021. BRAKER2: automatic eukaryotic genome annotation with GeneMark-EP+ and AUGUSTUS supported by a protein database. NAR Genom Bioinform [Internet] 3:1-11.
Brůna T, Lomsadze A, Borodovsky M. 2020. GeneMark-EP+: eukaryotic gene prediction with self-training in the space of genes and proteins. NAR Genom Bioinform [Internet] 2.
Bukhman Y V., DiPiazza N W, Piotrowski J, Shao J, Halstead A G W, Bui M D, Xie E, Sato T K. 2015. Modeling Microbial Growth Curves with GCAT. Bioenergy Res 8:1022-1030.
Camacho C, Coulouris G, Avagyan V, Ma N, Papadopoulos J, Bealer K, Madden T L. 2009. BLAST+: Architecture and applications. BMC Bioinformatics 10:1-9.
Campbell E, Kaltenbach M, Correy G J, Carr P D, Porebski B T, Livingstone E K, Afriat-Jurnou L, Buckle A M, Weik M, Hollfelder F, et al. 2016. The role of protein dynamics in the evolution of new enzyme function. Nature Chemical Biology 2016 12:11 [Internet] 12:944-950.
Capella-Gutiérrez S, Silla-Martínez J M, Gabaldón T. 2009. trimAl: a tool for automated alignment trimming in large-scale phylogenetic analyses. Bioinformatics 25:1972-1973.
Casa-Villegas M, Polaina J, Marín-Navarro J. 2018. Cellobiose fermentation by Saccharomyces cerevisiae: Comparative analysis of intra versus extracellular sugar hydrolysis. Process Biochemistry [Internet] 75:59-67.
Cheng C H C. 1998. Evolution of the diverse antifreeze proteins. Curr Opin Genet Dev 8:715 720.
Cheng Q, Michels C A. 1991. MAL11 and MAL61 encode the inducible high-affinity maltose transporter of Saccharomyces cerevisiae. J Bacteriol [Internet] 173:1817-1820.
Christin P-A, Salamin N, Savolainen V, Duvall M R, Besnard G. 2007. C4 Photosynthesis Evolved in Grasses via Parallel Adaptive Genetic Changes. Current Biology [Internet] 17:1241-1247.
Ciccarelli F D, von Mering C, Suyama M, Harrington E D, Izaurralde E, Bork P. 2005. Complex genomic rearrangements lead to novel primate gene function. Genome Res [Internet] 15:343-351.
Clark A G, Glanowski S, Nielsen R, Thomas P D, Kejariwal A, Todd M A, Tanenbaum D M, Civello D, Lu F, Murphy B, et al. 2003. Inferring Nonneutral Evolution from Human-Chimp-Mouse Orthologous Gene Trios. Science (1979) [Internet] 302:1960-1963.
Colomer M S, Chailyan A, Fennessy R T, Olsson K F, Johnsen L, Solodovnikova N, Forster J. 2020. Assessing Population Diversity of Brettanomyces Yeast Species and Identification of Strains for Brewing Applications. Front Microbiol [Internet] 11:495404.
Copley S D. 2012. Toward a systems biology perspective on enzyme evolution. Journal of Biological Chemistry [Internet] 287:3-10.
Cornette J L, Cease K B, Margalit H, Spouge J L, Berzofsky J A, DeLisi C. 1987. Hydrophobicity scales and computational techniques for detecting amphipathic structures in proteins. J Mol Biol 195:659-685.
Cousseau F E M, Alves S L, Trichez D, Stambuk B U. 2013. Characterization of maltotriose transporters from the Saccharomyces eubayanus subgenome of the hybrid Saccharomyces pastorianus lager brewing yeast strain Weihenstephan 34/70. Lett Appl Microbiol [Internet] 56:21-29.
Crandall J G, Fisher K J, Sato T K, Hittinger C T. 2023. Ploidy evolution in a wild yeast is linked to an interaction between cell type and metabolism. PLOS Biol [Internet] 21:e3001909.
Cubillos F A, Gibson B, Grijalva-Vallejos N, Krogerus K, Nikulin J. 2019. Bioprospecting for brewers: Exploiting natural diversity for naturally diverse beers. Yeast [Internet] 36:383-398.
Cui Y, Wong W H, Bornberg-Bauer E, Chan H S. 2002. Recombinatoric exploration of novel folded structures: A heteropolymer-based model of protein evolutionary landscapes. Proc Natl Acad Sci USA [Internet] 99:809-814.
David K T, Schraiber J G, Crandall J G, Labella A L, Opulente D A, Harrison M-C, Wolters J F, Zhou X, Shen X-X, Groenewald M, et al. 2024. Convergent expansions of keystone gene families drive metabolic innovation in a major eukaryotic clade. bioRxiv [Internet]: 2024.07.22.604484.
Day R E, Higgins V J, Rogers P J, Dawes I W. 2002. Characterization of the putative maltose transporters encoded by YDL247w and YJR160c. Yeast [Internet] 19:1015-1027.
Deng D, Sun P, Yan C, Ke M, Jiang X, Xiong L, Ren W, Hirata K, Yamamoto M, Fan S, et al. 2015. Molecular basis of ligand recognition and transport by glucose transporters. Nature [Internet] 526:391-396.
Deng D, Xu C, Sun P, Wu J, Yan C, Hu M, Yan N. 2014. Crystal structure of the human glucose transporter GLUT1. Nature [Internet] 510:121-125.
Dietvorst J, Londesborough J, Steensma H Y. 2005. Maltotriose utilization in lager yeast strains: MTTI encodes a maltotriose transporter. Yeast 22:775-788.
Donzella L, Sousa M J, Morrissey J P. 2023. Evolution and functional diversification of yeast sugar transporters. Essays Biochem [Internet] 67:811-827.
Dorus S, Vallender E J, Evans P D, Anderson J R, Gilbert S L, Mahowald M, Wyckoff G J, Malcom C M, Lahn B T. 2004. Accelerated evolution of nervous system genes in the origin of Homo sapiens. Cell [Internet] 119:1027-1040.
Drew D, North R A, Nagarathinam K, Tanabe M. 2021. Structures and General Transport Mechanisms by the Major Facilitator Superfamily (MFS). Chem Rev [Internet] 121:5289-5335.
Duval E H, Alves S L, Dunn B, Sherlock G, Stambuk B U. 2010. Microarray karyotyping of maltose-fermenting Saccharomyces yeasts with differing maltotriose utilization profiles reveals copy number variation in genes involved in maltose and maltotriose utilization. J Appl Microbiol 109:248-259.
Eisenberg D, Schwarz E, Komaromy M, Wall R. 1984. Analysis of membrane and surface protein sequences with the hydrophobic moment plot. J Mol Biol 179:125-142.
Engelman D M, Steitz T A, Goldman A. 1986. Identifying nonpolar transbilayer helices in amino acid sequences of membrane proteins. Annu Rev Biophys Biophys Chem 15:321-353.
Fairhead C, Dujon B. 2006. Structure of Kluyveromyces lactis subtelomeres: duplications and gene content. FEMS Yeast Res [Internet] 6:428-441.
Farwick A, Bruder S, Schadeweg V, Oreb M, Boles E. 2014. Engineering of yeast hexose transporters to transport <scp>d</scp>-xylose without inhibition by <scp>d</scp>-glucose. Proceedings of the National Academy of Sciences [Internet] 111:5159-5164.
Gallone B, Mertens S, Gordon J L, Maere S, Verstrepen K J, Steensels J. 2018. Origins, evolution, domestication and diversity of Saccharomyces beer yeasts. Curr Opin Biotechnol [Internet] 49:148-155.
Gibson B, Geertman J-MA, Hittinger C T, Krogerus K, Libkind D, Louis E J, Magalhães F, Sampaio J P. 2017. New yeasts-new brews: modern approaches to brewing yeast design and development. FEMS Yeast Res [Internet] 17:1-13.
Gietz R D, Schiestl R H. 2007. High-efficiency yeast transformation using the LiAc/SS carrier DNA/PEG method. Nat Protoc 2:31-34.
Gong L I, Suchard M A, Bloom J D. 2013. Stability-mediated epistasis constrains the evolution of an influenza protein. Elife 2013.
Gordon J L, Byrne K P, Wolfe K H. 2009. Additions, Losses, and Rearrangements on the Evolutionary Route from a Reconstructed Ancestor to the Modern Saccharomyces cerevisiae Genome. PLOS Genet [Internet] 5:e1000485.
Grantham R. 1974. Amino Acid Difference Formula to Help Explain Protein Evolution. Science (1979) 185:862-864.
Groenewald M, Hittinger C T, Bensch K, Opulente D A, Shen X X, Li Y, Liu C, LaBella A L, Zhou X, Limtong S, et al. 2023. A genome-informed higher rank classification of the biotechnologically important fungal subphylum Saccharomycotina. Stud Mycol 105:1-22.
Grosdidier A, Zoete V, Michielin O. 2011. SwissDock, a protein-small molecule docking web service based on EADock DSS. Nucleic Acids Res 39: W270-W277.
Guan L, Hariharan P. 2021. X-ray crystallography reveals molecular recognition mechanism for sugar binding in a melibiose transporter MelB. Commun Biol [Internet] 4:931.
Guan L, Kaback H R. 2006. LESSONS FROM LACTOSE PERMEASE. Annu Rev Biophys Biomol Struct [Internet] 35:67-91.
Guan L, Mirza O, Verner G, Iwata S, Kaback H R. 2007. Structural determination of wild-type lactose permease. Proceedings of the National Academy of Sciences [Internet] 104:15294-15298.
Gyurchev N Y, Coral-Medina A, Weening S M, Almayouf S, Kuijpers N G A, Nevoigt E, Louis E J. 2022. Beyond Saccharomyces pastorianus for modern lager brews: Exploring non-cerevisiae Saccharomyces hybrids with heterotic maltotriose consumption and novel aroma profile. Front Microbiol 13:1025132.
Ha S-J, Kim H, Lin Y, Jang M-U, Galazka J M, Kim T-J, Cate J H D, Jin Y-S. 2013. Single Amino Acid Substitutions in HXT2.4 from Scheffersomyces stipitis Lead to Improved Cellobiose Fermentation by Engineered Saccharomyces cerevisiae. Appl Environ Microbiol [Internet] 79:1500-1507.
Han E-K, Cotty F, Sottas C, Jiang H, Michels C A. 1995. Characterization of AGT1 encoding a general alpha-glucoside transporter from Saccharomyces. Mol Microbiol [Internet] 17:1093-1107.
HANSEN TF, CARTER AJR, CHIU C-H. 2000. Gene Conversion may aid Adaptive Peak Shifts. J Theor Biol [Internet] 207:495-511.
Hara K Y, Kobayashi J, Yamada R, Sasaki D, Kuriya Y, Hirono-Hara Y, Ishii J, Araki M, Kondo A. 2017. Transporter engineering in biomass utilization by yeast. FEMS Yeast Res [Internet] 17:61.
Harms M J, Thornton J W. 2014. Historical contingency and its biophysical basis in glucocorticoid receptor evolution. Nature [Internet] 512:203-207.
Hatanaka H, Toyonaga H, Ishida Y, Mizohata E, Ono E. 2022. Functional diversity and plasticity in the sugar preferences of Saccharomyces MALT transporters in domesticated yeasts. FEMS Yeast Res [Internet] 22:1-10.
Henderson R, Poolman B. 2017. Proton-solute coupling mechanism of the maltose transporter from Saccharomyces cerevisiae. Sci Rep [Internet] 7:14375.
Henikoff S, Henikoff J G. 1992. Amino acid substitution matrices from protein blocks. Proc Natl Acad Sci USA 89: 10915-10919.
Hittinger C T, Carroll S B. 2007. Gene duplication and the adaptive evolution of a classic genetic switch. Nature [Internet] 449:677-681.
Hittinger C T, Steele J L, Ryder D S. 2018. Diverse yeasts for diverse fermented beverages and foods. Curr Opin Biotechnol [Internet] 49:199-206.
Hoang D T, Chernomor O, von Haeseler A, Minh B Q, Vinh L S. 2018. UFBoot2: Improving the Ultrafast Bootstrap Approximation. Mol Biol Evol 35:518-522.
Hoekstra H E, Hirschmann R J, Bundey R A, Insel P A, Crossland J P. 2006. A single amino acid mutation contributes to adaptive beach mouse color pattern. Science (1979) [Internet] 313:101-104.
Hopp T P, Woods K R. 1983. A computer program for predicting protein antigenic determinants. Mol Immunol 20:483-489.
Horák J. 2013. Regulations of sugar transporters: insights from yeast. Curr Genet [Internet] 59:1-31.
Hughes A L. 1994. The evolution of functionally novel proteins after gene duplication. Proc R Soc Lond B Biol Sci [Internet] 256:119-124.
Hutzler M, Michel M, Kunz O, Kuusisto T, Magalhães F, Krogerus K, Gibson B. 2021. Unique Brewing-Relevant Properties of a Strain of Saccharomyces jurei Isolated From Ash (Fraxinus excelsior). Front Microbiol [Internet] 12.
Jabłońska J, Tawfik D S. 2022. Innovation and tinkering in the evolution of oxidases. Protein Science [Internet] 31:e4310.
JANIN J. 1979. Surface and inside volumes in globular proteins. Nature 277:491-492.
Jumper J, Evans R, Pritzel A, Green T, Figurnov M, Ronneberger O, Tunyasuvunakool K, Bates R, Žídek A, Potapenko A, et al. 2021. Highly accurate protein structure prediction with AlphaFold. Nature 2021 596:7873 596:583-589.
Kaback H R, Guan L. 2019. It takes two to tango: The dance of the permease. Journal of General Physiology [Internet] 151:878-886.
Kaltenbach M, Burke J R, Dindo M, Pabis A, Munsberg F S, Rabin A, Kamerlin S C L, Noel J P, Tawfik D S. 2018. Evolution of chalcone isomerase from a noncatalytic ancestor. Nat Chem Biol [Internet] 14:548-555.
Kalyaanamoorthy S, Minh B Q, Wong T K F, Von Haeseler A, Jermiin L S. 2017. ModelFinder: fast model selection for accurate phylogenetic estimates. Nature Methods 2017 14:6 14:587-589.
Kasahara M, Shimoda E, Maeda M. 1997. Amino Acid Residues Responsible for Galactose Recognition in Yeast Gal2 Transporter. Journal of Biological Chemistry [Internet] 272:16721-16724.
Kasahara T, Ishiguro M, Kasahara M. 2006. Eight Amino Acid Residues in Transmembrane Segments of Yeast Glucose Transporter Hxt2 Are Required for High Affinity Transport. Journal of Biological Chemistry 281:18532-18538.
Kasahara T, Kasahara M. 1998. Tryptophan 388 in Putative Transmembrane Segment 10 of the Rat Glucose Transporter Glut1 Is Essential for Glucose Transport. Journal of Biological Chemistry 273:29113-29117.
Kasahara T, Kasahara M. 2000. Three Aromatic Amino Acid Residues Critical for Galactose Transport in Yeast Gal2 Transporter. Journal of Biological Chemistry 275:4422-4428.
Kasahara T, Kasahara M. 2010. Identification of a Key Residue Determining Substrate Affinity in the Yeast Glucose Transporter Hxt7. Journal of Biological Chemistry [Internet] 285:26263-26268.
Kasahara T, Maeda M, Boles E, Kasahara M. 2009. Identification of a key residue determining substrate affinity in the human glucose transporter GLUT1. Biochimica et Biophysica Acta (BBA)—Biomembranes 1788:1051-1055.
Kasahara T, Maeda M, Ishiguro M, Kasahara M. 2007. Identification by Comprehensive Chimeric Analysis of a Key Residue Responsible for High Affinity Glucose Transport by Yeast HXT2. Journal of Biological Chemistry 282:13146-13150.
Kasahara T, Shimogawara K, Kasahara M. 2011. Crucial Effects of Amino Acid Side Chain Length in Transmembrane Segment 5 on Substrate Affinity in Yeast Glucose Transporter Hxt7. Biochemistry [Internet] 50:8674-8681.
Katoh K, Kuma K I, Toh H, Miyata T. 2005. MAFFT version 5: improvement in accuracy of multiple sequence alignment. Nucleic Acids Res 33:511-518.
Katoh K, Misawa K, Kuma K I, Miyata T. 2002. MAFFT: a novel method for rapid multiple sequence alignment based on fast Fourier transform. Nucleic Acids Res 30:3059-3066.
Katoh K, Standley D M. 2013. MAFFT Multiple Sequence Alignment Software Version 7: Improvements in Performance and Usability. Mol Biol Evol 30:772-780.
Kauffman S, Levin S. 1987. Towards a general theory of adaptive walks on rugged landscapes. J Theor Biol [Internet] 128:11-45.
Kelley L A, Mezulis S, Yates C M, Wass M N, Sternberg M J E. 2015. The Phyre2 web portal for protein modeling, prediction and analysis. Nat Protoc 10:845-858.
Kim H, Oh E J, Lane S T, Lee W H, Cate J H D, Jin Y S. 2018. Enhanced cellobiose fermentation by engineered Saccharomyces cerevisiae expressing a mutant cellodextrin facilitator and cellobiose phosphorylase. J Biotechnol 275:53-59.
Kriventseva E V., Kuznetsov D, Tegenfeldt F, Manni M, Dias R, Simão FA, Zdobnov E M. 2019. OrthoDB v10: sampling the diversity of animal, plant, fungal, protist, bacterial and viral genomes for evolutionary and functional annotations of orthologs. Nucleic Acids Res [Internet] 47: D807-D811.
Kuang M C, Kominek J, Alexander W G, Cheng J-F, Wrobel R L, Hittinger C T. 2018. Repeated Cis-Regulatory Tuning of a Metabolic Bottleneck Gene during Evolution. Wittkopp P, editor. Mol Biol Evol [Internet] 35:1968-1981.
Kyte J, Doolittle R F. 1982. A simple method for displaying the hydropathic character of a protein. J Mol Biol 157:105-132.
Langdon Q K, Peris D, Eizaguirre J I, Opulente D A, Buh K V., Sylvester K, Jarzyna M, Rodríguez ME, Lopes C A, Libkind D, et al. 2020. Postglacial migration shaped the genomic diversity and global distribution of the wild ancestor of lager-brewing hybrids. PLOS Genet [Internet] 16:e1008680.
Lee S B, Tremaine M, Place M, Liu L, Pier A, Krause D J, Xie D, Zhang Y, Landick R, Gasch A P, et al. 2021. Crabtree/Warburg-like aerobic xylose fermentation by engineered Saccharomyces cerevisiae. Metab Eng 68:119-130.
Leffler E M, Band G, Busby G B J, Kivinen K, Le Q S, Clarke G M, Bojang K A, Conway D J, Jallow M, Sisay-Joof F, et al. 2017. Resistance to malaria through structural variation of red blood cell invasion receptors. Science (1979) [Internet] 356:1140-1152.
Letunic I, Bork P. 2021. Interactive Tree Of Life (iTOL) v5: an online tool for phylogenetic tree display and annotation. Nucleic Acids Res 49: W293-W296.
Li X C, Fay J C. 2019. Multiple changes underlie allelic divergence of CUP2 between Saccharomyces species. G3: Genes, Genomes, Genetics 9:3595-3600.
Libkind D, Hittinger C T, Valério E, Gonçalves C, Dover J, Johnston M, Gonçalves P, Sampaio J P. 2011. Microbe domestication and the identification of the wild genetic stock of lager-brewing yeast. Proceedings of the National Academy of Sciences [Internet] 108:14539-14544.
Liu H, Maclean C J, Zhang J. 2019. Evolution of the Yeast Recombination Landscape. Mol Biol Evol [Internet] 36:412-422.
Long M, Betrán E, Thornton K, Wang W. 2003. The origin of new genes: glimpses from the young and old. Nature Reviews Genetics 2003 4:11 [Internet] 4:865-875.
Long M, Langley C H. 1993. Natural Selection and the Origin of jingwei, a Chimeric Processed Functional Gene in Drosophila. Science (1979) [Internet] 260:91-95.
Lunzer M, Miller S P, Felsheim R, Dean A M. 2005. Evolution: The biochemical architecture of an ancient adaptive landscape. Science (1979) [Internet] 310:499-501.
Lynch M. 2005. Simple evolutionary pathways to complex proteins. Protein Science [Internet] 14:2217-2225.
Manni M, Berkeley M R, Seppey M, Simão FA, Zdobnov E M. 2021. BUSCO Update: Novel and Streamlined Workflows along with Broader and Deeper Phylogenetic Coverage for Scoring of Eukaryotic, Prokaryotic, and Viral Genomes. Mol Biol Evol [Internet] 38:4647-4654.
Des Marais D L, Rausher M D. 2008. Escape from adaptive conflict after duplication in an anthocyanin pathway gene. Nature 454:762-765.
Marger M D, Saier M H. 1993. A major superfamily of transmembrane facilitators that catalyse uniport, symport and antiport. Trends Biochem Sci [Internet] 18:13-20.
Maynard Smith J. 1970. Natural Selection and the Concept of a Protein Space. Nature 225:563-564
Mefford H C, Trask B J. 2002. The complex structure and dynamic evolution of human subtelomeres. Nature Reviews Genetics 2002 3:2 [Internet] 3:91-102.
Meger A T, Spence M A, Sandhu M, Matthews D, Chen J, Jackson C J, Raman S. 2024. Rugged fitness landscapes minimize promiscuity in the evolution of transcriptional repressors. Cell Syst [Internet] 15:374-387.e6.
Méheust R, Bhattacharya D, Pathmanathan J S, McInerney J O, Lopez P, Bapteste E. 2018. Formation of chimeric genes with essential functions at the origin of eukaryotes. BMC Biol [Internet] 16:30.
Metzger B P, Park Y, Starr T N, Thornton J W. 2024. Epistasis facilitates functional evolution in an ancient transcription factor. Elife [Internet] 12.
Meussdorfer F, Zarnkow M. 2009. Starchy Raw Materials. In: Esslinger H M, editor. Handbook of Brewing: Process, Technology, Markets. Weinheim: Wiley-VCH. p. 43-83.
Minh B Q, Schmidt H A, Chernomor O, Schrempf D, Woodhams M D, Von Haeseler A, Lanfear R, Teeling E. 2020. IQ-TREE 2: New Models and Efficient Methods for Phylogenetic Inference in the Genomic Era. Mol Biol Evol 37:1530-1534.
Miyazaki K, Arnold F H. 1999. Exploring nonnatural evolutionary pathways by saturation mutagenesis: Rapid improvement of protein function. J Mol Evol [Internet] 49:716-720.
Nielsen R, Bustamante C, Clark A G, Glanowski S, Sackton T B, Hubisz M J, Fledel-Alon A, Tanenbaum D M, Civello D, White T J, et al. 2005. A Scan for Positively Selected Genes in the Genomes of Humans and Chimpanzees. PLOS Biol [Internet] 3:e170.
Nijland J G, Driessen A J M. 2020. Engineering of Pentose Transport in Saccharomyces cerevisiae for Biotechnological Applications. Front Bioeng Biotechnol [Internet] 7:464.
Nijland J G, Shin H Y, de Waal P P, Klaassen P, Driessen A J M. 2018. Increased xylose affinity of Hxt2 through gene shuffling of hexose transporters in Saccharomyces cerevisiae. J Appl Microbiol [Internet] 124:503-510.
Nishikawa K K, Hoppe N, Smith R, Bingman C, Raman S. 2021. Epistasis shapes the fitness landscape of an allosteric specificity switch. Nat Commun [Internet] 12:5562.
Nurminsky D I, Nurminskaya M V., De Aguiar D, Hartl D L. 1998. Selective sweep of a newly evolved sperm-specific gene in Drosophila. Nature 1998 396:6711 [Internet] 396:572-575.
O'Donnell S, Yue J-X, Saada O A, Agier N, Caradec C, Cokelaer T, De Chiara M, Delmas S, Dutreux F, Fournier T, et al. 2023. Telomere-to-telomere assemblies of 142 strains characterize the genome structural landscape in Saccharomyces cerevisiae. Nat Genet [Internet] 55:1390-1399.
Oh E J, Jin Y S. 2020. Engineering of Saccharomyces cerevisiae for efficient fermentation of cellulose. FEMS Yeast Res 20.
Oh E J, Kwak S, Kim H, Jin Y-S. 2017. Transporter engineering for cellobiose fermentation under lower pH conditions by engineered Saccharomyces cerevisiae. Bioresour Technol [Internet] 245:1469-1475.
Opulente D A, LaBella A L, Harrison M-C, Wolters J F, Liu C, Li Yonglin, Kominek J, Steenwyk J L, Stoneman H R, VanDenAvond J, et al. 2024. Genomic factors shape carbon and nitrogen metabolic niche breadth across Saccharomycotina yeasts. Science (1979) [Internet] 384.
Ortlund E A, Bridgham J T, Redinbo M R, Thornton J W. 2007. Crystal Structure of an Ancient Protein: Evolution by Conformational Epistasis. Science (1979) [Internet] 317:1544-1548.
Pao S S, Paulsen I T, Saier M H. 1998. Major Facilitator Superfamily. Microbiology and Molecular Biology Reviews [Internet] 62:1-34.
Park Y, Metzger B P H, Thornton J W. 2022. Epistatic drift causes gradual decay of predictability in protein evolution. Science (1979) [Internet] 376:823-830.
Patthy L. 2003. Modular assembly of genes and the evolution of new functions. Genetica [Internet] 118:217-231.
Paulsen P A, Custódio TF, Pedersen B P. 2019. Crystal structure of the plant symporter STP10 illuminates sugar uptake mechanism in monosaccharide transporter superfamily. Nat Commun [Internet] 10.
Peris D, Ubbelohde E J, Kuang M C, Kominek J, Langdon Q K, Adams M, Koshalek J A, Hulfachor A B, Opulente D A, Hall D J, et al. 2023. Macroevolutionary diversity of traits and genomes in the model yeast genus Saccharomyces. Nat Commun [Internet] 14:690.
Peris D, Sylvester K, Libkind D, Gonçalves P, Sampaio J P, Alexander W G, Hittinger C T. 2014. Population structure and reticulate evolution of Saccharomyces eubayanus and its lager-brewing hybrids. Molecular Ecology 23 (8), 2031-2045.
Peter J, De Chiara M, Friedrich A, Yue J-X, Pflieger D, Bergström A, Sigwalt A, Barre B, Freel K, Llored A, et al. 2018. Genome evolution across 1,011 Saccharomyces cerevisiae isolates. Nature [Internet] 556:339-344.
Podgornaia A I, Laub M T. 2015. Pervasive degeneracy and epistasis in a protein-protein interface. Science (1979) [Internet] 347:673-677.
Poelwijk F J, Kiviet D J, Weinreich D M, Tans S J. 2007. Empirical fitness landscapes reveal accessible evolutionary paths. Nature 2006 445:7126 [Internet] 445:383-386.
Pokusaeva V O, Usmanova D R, Putintseva E V., Espinar L, Sarkisyan K S, Mishin A S, Bogatyreva N S, Ivankov D N, Akopyan A V., Avvakumov S Y, et al. 2019. An experimental assay of the interactions of amino acids from orthologous sequences shaping a complex fitness landscape. PLOS Genet [Internet] 15:e1008079.
Pougach K, Voet A, Kondrashov F A, Voordeckers K, Christiaens J F, Baying B, Benes V, Sakai R, Aerts J, Zhu B, et al. 2014. Duplication of a promiscuous transcription factor drives the emergence of a new regulatory network. Nat Commun [Internet] 5:1-11
Projecto-Garcia J, Natarajan C, Moriyama H, Weber R E, Fago A, Cheviron Z A, Dudley R, McGuire J A, Witt C C, Storz J F. 2013. Repeated elevational transitions in hemoglobin function during the evolution of Andean hummingbirds. Proc Natl Acad Sci USA [Internet] 110:20669-20674.
Quistgaard E M, Low C, Guettou F, Nordlund P. 2016. Understanding transport by the major facilitator superfamily (MFS): structures pave the way. Nat Rev Mol Cell Biol [Internet] 17:123-132.
Roach M J, Borneman A R. 2020. New genome assemblies reveal patterns of domestication and adaptation across Brettanomyces (Dekkera) species. BMC Genomics [Internet] 21:1-14.
Rogers R L, Bedford T, Lyons A M, Hartl D L. 2010. Adaptive impact of the chimeric gene Quetzalcoatl in Drosophila melanogaster. Proceedings of the National Academy of Sciences [Internet] 107:10943-10948.
Rogers R L, Hartl D L. 2012. Chimeric Genes as a Source of Rapid Evolution in Drosophila melanogaster. Mol Biol Evol [Internet] 29:517-529.
Rose G D, Geselowitz A R, Lesser G J, Lee R H, Zehfus M H. 1985. Hydrophobicity of Amino Acid Residues in Globular Proteins. Science (1979) 229:834-838.
Rozpedowska E, Hellborg L, Ishchuk O P, Orhan F, Galafassi S, Merico A, Woolfit M, Compagno C, Piškur J. 2011. Parallel evolution of the make-accumulate-consume strategy in Saccharomyces and Dekkera yeasts. Nature Communications 2011 2:1 [Internet] 2:1-7.
de Ruijter J C, Igarashi K, Penttilä M. 2020. The Lipomyces starkeyi gene Ls120451 encodes a cellobiose transporter that enables cellobiose fermentation in Saccharomyces cerevisiae. FEMS Yeast Res [Internet] 20.
Saier M H. 2000. Families of transmembrane sugar transport proteins. Mol Microbiol [Internet] 35:699-710.
Saier M H, Reddy V S, Moreno-Hagelsieb G, Hendargo K J, Zhang Y, Iddamsetty V, Lam K J K, Tian N, Russum S, Wang J, et al. 2021. The Transporter Classification Database (TCDB): 2021 update. Nucleic Acids Res [Internet] 49: D461-D467.
Saier M H, Tran C V., Barabote R D. 2006. TCDB: the Transporter Classification Database for membrane transport protein analyses and information. Nucleic Acids Res [Internet] 34: D181-D186.
Salema-Oom M, Valadão Pinto V, Gonçalves P, Spencer-Martins I. 2005. Maltotriose Utilization by Industrial Saccharomyces Strains: Characterization of a New Member of the α-Glucoside Transporter Family. Appl Environ Microbiol [Internet] 71:5044-5049.
Sarkisyan K S, Bolotin D A, Meer M V., Usmanova D R, Mishin A S, Sharonov G V., Ivankov D N, Bozhanova N G, Baranov M S, Soylemez O, et al. 2016. Local fitness landscape of the green fluorescent protein. Nature 2015 533:7603 [Internet] 533:397-401.
Serra Colomer M, Funch B, Forster J. 2019. The raise of Brettanomyces yeast species for beer production. Curr Opin Biotechnol 56:30-35.
Shah P, McCandlish D M, Plotkin J B. 2015. Contingency and entrenchment in protein evolution under purifying selection. Proc Natl Acad Sci USA [Internet] 112: E3226-E3235.
Shen X-X, Opulente D A, Kominek J, Zhou X, Steenwyk J L, Buh K V., Haase M A B, Wisecaver J H, Wang M, Doering D T, et al. 2018. Tempo and Mode of Genome Evolution in the Budding Yeast Subphylum. Cell 175:1533-1545.e20.
Shen X X, Opulente D A, Kominek J, Zhou X, Steenwyk J L, Buh K V., Haase M A B, Wisecaver J H, Wang M, Doering D T, et al. 2018. Tempo and Mode of Genome Evolution in the Budding Yeast Subphylum. Cell 175:1533-1545.e20.
Smithers B, Oates M, Gough J. 2019. ‘Why genes in pieces?’—revisited. Nucleic Acids Res [Internet] 47:4970-4973.
Soylemez O, Kondrashov F A. 2012. Estimating the Rate of Irreversibility in Protein Evolution. Genome Biol Evol [Internet] 4:1213-1222.
Sprouffske K, Wagner A. 2016. Growthcurver: an R package for obtaining interpretable metrics from microbial growth curves. BMC Bioinformatics 17:172.
Srikant S, Gaudet R, Murray A W. 2020. Selecting for Altered Substrate Specificity Reveals the Evolutionary Flexibility of ATP-Binding Cassette Transporters. Current Biology 30:1689-1702.e6.
Stambuk B U, Araujo P S. 2001. Kinetics of active alpha-glucoside transport in Saccharomyces cerevisiae. FEMS Yeast Res [Internet] 1:73-78.
Stambuk B U, Batista A S, De Araujo P S. 2000. Kinetics of active sucrose transport in Saccharomyces cerevisiae. J Biosci Bioeng [Internet] 89:212-214.
Stambuk B U, Silva M A, Panek A D, Araujo P S. 1999. Active Î±-glucoside transport in Saccharomyces cerevisiae. FEMS Microbiol Lett [Internet] 170:105-110.
Stanke M, Diekhans M, Baertsch R, Haussler D. 2008. Using native and syntenically mapped cDNA alignments to improve de novo gene finding. Bioinformatics [Internet] 24:637-644.
Starr T N, Flynn J M, Mishra P, Bolon DNA, Thornton J W. 2018. Pervasive contingency and entrenchment in a billion years of Hsp90 evolution. Proceedings of the National Academy of Sciences [Internet] 115:4453-4458.
Starr T N, Picton L K, Thornton J W. 2017. Alternative evolutionary histories in the sequence space of an ancient protein. Nature [Internet] 549:409-413.
Starr T N, Thornton J W. 2016. Epistasis in protein evolution. Protein Science [Internet] 25:1204-1218.
Steindel P A, Chen E H, Wirth J D, Theobald D L. 2016. Gradual neofunctionalization in the convergent evolution of trichomonad lactate and malate dehydrogenases. Protein Science [Internet] 25:1319-1331.
Sun L, Zeng X, Yan C, Sun X, Gong X, Rao Y, Yan N. 2012. Crystal structure of a bacterial homologue of glucose transporters GLUT1-4. Nature [Internet] 490:361-366.
Tawfik O K and DS. 2010. Enzyme Promiscuity: A Mechanistic and Evolutionary Perspective. Annu Rev Biochem [Internet] 79:471-505.
Townsend J P, Nielsen K M, Fishert D S, Hartl D L. 2003. Horizontal Acquisition of Divergent Chromosomal DNA in Bacteria: Effects of Mutator Phenotypes. Genetics [Internet] 164:13-21.
Trichez D, Knychala M M, Figueiredo C M, Alves S L, da Silva M A, Miletti L C, de Araujo P S, Stambuk B U. 2019. Key amino acid residues of the AGT1 permease required for maltotriose consumption and fermentation by Saccharomyces cerevisiae. J Appl Microbiol [Internet] 126:580-594.
Turner T L, Kim H, Kong I I, Liu J-J, Zhang G-C, Jin Y-S. 2016. Engineering and Evolution of Saccharomyces cerevisiae to Produce Biofuels and Chemicals. In: Advances in Biochemical Engineering/Biotechnology. Vol. 162. Springer, Cham. p. 175-215.
De Visser JAGM, Krug J. 2014. Empirical fitness landscapes and the predictability of evolution. Nature Reviews Genetics 2014 15:7 [Internet] 15:480-490.
Voordeckers K, Brown C A, Vanneste K, van der Zande E, Voet A, Maere S, Verstrepen K J. 2012. Reconstruction of Ancestral Metabolic Enzymes Reveals Molecular Mechanisms Underlying Evolutionary Innovation through Gene Duplication. PLOS Biol 10.
Wang S C, Davejan P, Hendargo K J, Javadi-Razaz I, Chou A, Yee D C, Ghazi F, Lam K J K, Conn A M, Madrigal A, et al. 2020. Expansion of the Major Facilitator Superfamily (MFS) to include novel transporters as well as transmembrane-acting enzymes. Biochimica et Biophysica Acta (BBA)—Biomembranes [Internet] 1862:183277.
Wang W, Zhang J, Alvarez C, Llopart A, Long M. 2000. The Origin of the Jingwei Gene and the Complex Modular Structure of Its Parental Gene, Yellow Emperor, in Drosophila melanogaster. Mol Biol Evol [Internet] 17:1294-1301.
Waterhouse A, Bertoni M, Bienert S, Studer G, Tauriello G, Gumienny R, Heer F T, de Beer T A P, Rempfer C, Bordoli L, et al. 2018. SWISS-MODEL: homology modelling of protein structures and complexes. Nucleic Acids Res 46: W296-W303.
Waterhouse R M, Seppey M, Simao F A, Manni M, Ioannidis P, Klioutchnikov G, Kriventseva E V., Zdobnov E M. 2018. BUSCO Applications from Quality Assessments to Gene Prediction and Phylogenomics. Mol Biol Evol [Internet] 35:543-548.
Weinreich D M, Delaney N F, DePristo M A, Hartl D L. 2006. Darwinian Evolution Can Follow Only Very Few Mutational Paths to Fitter Proteins. Science (1979) [Internet] 312:111-114.
Weinreich D M, Lan Y, Wylie C S, Heckendorn R B. 2013. Should evolutionary geneticists worry about higher-order epistasis? Curr Opin Genet Dev 23:700-707.
Weinreich D M, Watson R A, Chao L. 2005. PERSPECTIVE: SIGN EPISTASIS AND GENETIC COSTRAINT ON EVOLUTIONARY TRAJECTORIES. Evolution (N Y) [Internet] 59:1165-1174.
Wheeler L C, Harms M J. 2021. Were Ancestral Proteins Less Specific? Malik H, editor. Mol Biol Evol [Internet] 38:2227-2239.
Wheeler L C, Lim S A, Marqusee S, Harms M J. 2016. The thermostability and specificity of ancient proteins. Curr Opin Struct Biol 38:37-43.
Wieczorke R, Krampe S, Weierstall T, Freidel K, Hollenberg C P, Boles E. 1999. Concurrent knock-out of at least 20 transporter genes is required to block uptake of hexoses in Saccharomyces cerevisiae. FEBS Lett [Internet] 464:123-128.
Wong E D, Miyasato S R, Aleksander S, Karra K, Nash R S, Skrzypek M S, Weng S, Engel S R, Cherry J M. 2023. Saccharomyces genome database update: server architecture, pan-genome nomenclature, and external resources. Genetics [Internet] 224:191.
Woolfit M, Rozpędowska E, Piškur J, Wolfe K H. 2007. Genome survey sequencing of the wine spoilage yeast Dekkera (Brettanomyces) bruxellensis. Eukaryot Cell [Internet] 6:721-733.
Wright S. 1931. EVOLUTION IN MENDELIAN POPULATIONS. Genetics [Internet] 16:97-159.
Wright S. 1932. The roles of mutation, inbreeding, crossbreeding, and selection in evolution. Proceedings of the 6th International Congress of Genetics 1:356-366.
Wu N C, Dai L, Olson C A, Lloyd-Smith J O, Sun R. 2016. Adaptation in protein fitness landscapes is facilitated by indirect paths. Elife 5.
Xia X, Li W H. 1998. What amino acid properties affect protein evolution? J Mol Evol 47:557-564.
Xie V C, Pu J, Metzger B P H, Thornton J W, Dickinson B C. 2021. Contingency and chance erase necessity in the experimental evolution of ancestral proteins. Elife [Internet] 10:1-87. Yang J, Yan R, Roy A, Xu D, Poisson J, Zhang Y. 2015. The I-TASSER Suite: protein structure and function prediction. Nat Methods 12:7-8.
Yi X, Dean A M. 2019. Adaptive Landscapes in the Age of Synthetic Biology. Mol Biol Evol [Internet] 36:890-907.
Yokoyama S, Tada T, Zhang H, Britt L. 2008. Elucidation of phenotypic adaptations: Molecular analyses of dim-light vision proteins in vertebrates. Proc Natl Acad Sci USA [Internet] 105:13480-13485.
Young E M, Tong A, Bui H, Spofford C, Alper H S. 2014. Rewiring yeast sugar transporter preference through modifying a conserved protein motif. Proceedings of the National Academy of Sciences [Internet] 111:131-136.
Yue J-X, Li J, Aigrain L, Hallin J, Persson K, Oliver K, Bergström A, Coupland P, Warringer J, Lagomarsino M C, et al. 2017. Contrasting evolutionary genome dynamics between domesticated and wild yeasts. Nat Genet [Internet] 49:913-924.
Zastrow C R, Hollatz C, de Araujo P S, Stambuk B U. 2001. Maltotriose fermentation by Saccharomyces cerevisiae. J Ind Microbiol Biotechnol [Internet] 27:34-38.
Zhang J, Dean A M, Brunet F, Long M. 2004. Evolving protein functional diversity in new genes of Drosophila. Proceedings of the National Academy of Sciences [Internet] 101:16246-16250.
Zhang J, Zhang Y ping, Rosenberg H F. 2002. Adaptive evolution of a duplicated pancreatic ribonuclease gene in a leaf-eating monkey. Nature Genetics 2002 30:4 [Internet] 30:411-415.

What is claimed:

1. An engineered MalT4 protein comprising at least 3 amino acid substitutions at positions selected from residues 468, 503, 504, 505, 508, 512, and 379 of SEQ ID NO:1; wherein
a) the substitution at residue 468 is S468F;
b) the substitution at residue 503 is I503M;
c) the substitution at residue 504 is G504A;
d) the substitution at residue 505 is selected from N505C, N505S, and N505G;
e) the substitution at residue 508 is V508T;
f) the substitution at residue 512 is selected from I512T and I512V; and/or
g) the substitution at residue 379 is S379C;
wherein the engineered MalT4 protein has maltotriose transport activity.

2. The engineered MalT4 protein of claim 1, wherein the protein has at least 6 amino acid substitutions, and wherein the amino acid substitutions comprise:
a) S468F, I503M, G504A, and V508T;
b) a substitution at residue 505 selected from N505C, N505S, and N505G; and
c) a substitution at residue 512 selected from I512T and I512V.

3. The engineered MalT4 protein of claim 2, wherein the protein has at least 7 amino acid substitutions, and wherein the amino acid substitutions further comprise S379C.

4. The engineered MalT4 protein of claim 1 comprising the amino acid substitutions:
a) S468F, I503M, G504A, N505C, V508T, and I512T;
b) I503M, G504A, N505C, V508T, and I512T;
c) S468F, G504A, N505C, V508T, and I512T;
d) S468F, I503M, N505C, V508T, and I512T;
e) S468F, I503M, G504A, N505C, and I512T;
f) S468F, I503M, G504A, N505C, and V508T;
g) I503M, N505C, V508T, and I512T;
h) I503M, N505C, and I512T;
i) N505C, V508T, and I512T;
j) S468F, I503M, G504A, N505S, V508T, and I512T;
k) S468F, I503M, G504A, N505G, V508T, and I512T;
l) S468F, I503M, G504A, N505C, V508T, and I512V; or
m) S379C, S468F, I503M, G504A, N505C, V508T, and I512T.

5. The engineered MalT4 protein of claim 1, wherein the engineered MalT4 protein has increased maltotriose transport activity as compared to MalT434 (SEQ ID NO: 2).

6. A polynucleotide encoding the engineered MalT4 protein of claim 1.

7. The polynucleotide of claim 6, wherein the polynucleotide is codon-optimized for expression in a cell.

8. The polynucleotide of claim 7, wherein the cell is a yeast cell.

9. A construct comprising a promoter operably linked to the polynucleotide of claim 6.

10. The construct of claim 9, wherein the promoter is a heterologous promoter.

11. The construct of claim 9, wherein the promoter is a MalT4 promoter.

12. A vector comprising the polynucleotide of claim 6.

13. A Saccharomyces eubayanus yeast cell comprising the engineered MalT4 protein of claim 1.

14. The yeast cell of claim 13, wherein the yeast cell has increased maltotriose transport activity as compared to a yeast cell that expresses MalT434 (SEQ ID NO: 2).

15. The yeast cell of claim 13, wherein the yeast cell is strain yHKS210 or yHJC207.

16. A method for making a fermentation product comprising: culturing the yeast cell of claim 13 with a fermentable substrate to produce the fermentation product.

17. The method of claim 16, wherein the fermentable substrate comprises maltotriose.

18. The method of claim 17, wherein the fermentable substrate comprises wort, mash, dough, or malt extract.

19. The method of claim 16, wherein the fermentation product is beer, wine, or bread.

20. The method of claim 19, wherein the fermentation product is a lager.


SEQUENCE LISTING (191 sequences)

SEQ ID NO: 1 - protein, 613 aa, Saccharomyces eubayanus
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNIGNIVVAVLILYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 2 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMACIVTAVLTLYQLNSEKWDWGAKSGFFWGGLCFATLVWAVFDLPETAGRTFIEINELFRLGVPARKFKSTKVDPFAAAKAISDEINHKDPKEDMKASAEEREQSTPSLMD

SEQ ID NO: 3 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMACIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 4 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNIGNIVVAVLILYQLNSEKWDWGAKSGFFWGGLCFATLVWAVFDLPETAGRTFIEINELFRLGVPARKFKSTKVDPFAAAKAISDEINHKDPKEDMKASAEEREQSTPSLMD

SEQ ID NO: 5 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNMACIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 6 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNIACIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 7 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMGCIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 8 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMANIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 9 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMACIVVAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 10 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMACIVTAVLILYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 11 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNMGCIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 12 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNMGCIVVAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 13 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNIGCIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 14 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMANIVVAVLILYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 15 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFSYNLGIAPVVFCLVSEIPSSRLRTKSIILARNAYNMACIVVAVLILYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 16 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNIGNIVVAVLILYQLNSEKWDWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 17 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMASIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 18 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMAGIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 19 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGASLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMACIVTAVLVLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 20 - protein, 613 aa, synthetic construct
MKGLSSMINRKKCNGNSSSIETEGGFGAAECNSIELEEQGKKTDFDLAHLEYGQGPAALSENDEVTPNILDAAQDAKEADDSEREMPLMTALKTYPKAAAWSLLVSTTLIQEGYDTAILGSFYALPVFQKKYGSLNARTGEWEISVSWQIGLCLCYMAGEIVGLQLTGPSVDLMGNRYTLIMALMFLTAFIFILYFCKSLGMIAAGQALCGMPWGCFQCLTVSYASEICPMALRYYLTTYSNLCWLFGQLFAAGIMKNSQNKYADSDLGYTLPFALQWIWPVPLAIAIFFAPESPWWLVKKGRLEQAKRSLERTLSGKGAEKESLVAMELDKIKMTIEKEKKLSDDEGSYLDCLRGKVNRRRTRIACLCWAGQTVCGACLIGYSTYFYEKAGVSTETAFTFSIIQYCLGIAATFLSWWASKYYGRYDLYACGLAFQTVILFIIGGLGCSDTHGAKMGSGALLMVVAFFYNLGIAPVVFCLVSEIPSSRLRTKSIILARNAYNMACIVTAVLTLYQLNSEKWNWGAKSGFFWGGFCLAVLIWAVFDLPETAGRTFIEINELFRLGVPARKFKSTKVDPFAAAKAISDEINHKDPKEDMKASAEEREQSTPSLMD

SEQ ID NO: 21 - DNA, 58 bp, synthetic construct
  source 1..58: mol_type = other DNA
tgtatcgtaactgctgtcctgacattgtaccaactgaactctgagaaatggaactggg

SEQ ID NO: 22 - DNA, 59 bp, synthetic construct
  source 1..59: mol_type = other DNA
accccagttccatttctcagagttcagttggtacaatgtcaggacagcagttacgatac

SEQ ID NO: 23 - DNA, 58 bp, synthetic construct
  source 1..58: mol_type = other DNA
aatatcgtagtcgctgttttgattttgtaccaattgaattcagaaaaatgggattggg

SEQ ID NO: 24 - DNA, 57 bp, synthetic construct
  source 1..57: mol_type = other DNA
accccaatcccatttttctgaattcaattggtacaaaatcaaaacagcgactacgat

SEQ ID NO: 25 - DNA, 57 bp, synthetic construct
  source 1..57: mol_type = other DNA
aatgcccgtacaggagaatgggagatttcgtcagtttcttggcagattggtttatct

SEQ ID NO: 26 - DNA, 59 bp, synthetic construct
  source 1..59: mol_type = other DNA
taaagataaaccaatctgccaagaaactgacgaaatctcccattctcctgtacgggcat

SEQ ID NO: 27 - DNA, 59 bp, synthetic construct
  source 1..59: mol_type = other DNA
gatgaagtaacgccaaatattctcgacgctgctatgcaggatgcgaaggaggcagacga

SEQ ID NO: 28 - DNA, 60 bp, synthetic construct
  source 1..60: mol_type = other DNA
ttcgtctgcctccttcgcatcctgcatagcagcgtcgagaatatttggcgttacttcatc

SEQ ID NO: 29 - DNA, 59 bp, synthetic construct
  source 1..59: mol_type = other DNA
gatgaggaagtcccagatcttctcgatgaggcgcaggatgctaaggaggccgacgatag

SEQ ID NO: 30 - DNA, 60 bp, synthetic construct
  source 1..60: mol_type = other DNA
actatcgtcggcctccttagcatcctgcgcctcatcgagaagatctgggacttcctcatc

SEQ ID NO: 31 - DNA, 60 bp, synthetic construct
  source 1..60: mol_type = other DNA
aaaatgggaagtggcgctcttctaatggtcgttgcgttcttttacaacctggggattgcc

SEQ ID NO: 32 - DNA, 60 bp, synthetic construct
  source 1..60: mol_type = other DNA
gcaaaaaacgacaggggcaatccccaggttgtaaaagaacgcaacgaccattagaagagc

SEQ ID NO: 33 - DNA, 60 bp, synthetic construct
  source 1..60: mol_type = other DNA
aaaatgggaagtggcgctcttctaatggtcgttgcgttcttgtacaacctggggattgcc

SEQ ID NO: 34 - DNA, 60 bp, synthetic construct
  source 1..60: mol_type = other DNA
gcaaaaaacgacaggggcaatccccaggttgtacaagaacgcaacgaccattagaagagc

SEQ ID NO: 35 - DNA, 60 bp, synthetic construct
  source 1..60: mol_type = other DNA
agtcgctgttttgattttgtaccaactgaactctgagaaatgggattggggtgccaagtc

SEQ ID NO: 36 - DNA, 60 bp, synthetic construct
  source 1..60: mol_type = other DNA
gcagaatcctccccagaaaaagcctgacttggcaccccaatcccatttctcagagttcag

SEQ ID NO: 37 - DNA, 60 bp, synthetic construct
  source 1..60: mol_type = other DNA
agtcgctgttttgattttgtaccaactgaactctgagaaatgggactggggtgccaagtc

SEQ ID NO: 38 - DNA, 60 bp, synthetic construct
  source 1..60: mol_type = other DNA
gcagaatcctccccagaaaaagcctgacttggcaccccagtcccatttctcagagttcag

SEQ ID NO: 39 - DNA, 22 bp, synthetic construct
  source 1..22: mol_type = other DNA
cgatactaacgccgccatccag

SEQ ID NO: 40 - DNA, 20 bp, synthetic construct
  source 1..20: mol_type = other DNA
ccataatcttggccgcacgc

SEQ ID NO: 41 - DNA, 20 bp, synthetic construct
  source 1..20: mol_type = other DNA
cgtcaatcgtatgtgaatgc

SEQ ID NO: 42 - DNA, 20 bp, synthetic construct
  source 1..20: mol_type = other DNA
tacagatcttactgcgcaac

SEQ ID NO: 43 - DNA, 60 bp, synthetic construct
  source 1..60: mol_type = other DNA
cgggtggcgaatgggacttttacaggagaatgggagatttgttttagagctagaaatagc

SEQ ID NO: 44 - DNA, 60 bp, synthetic construct
  source 1..60: mol_type = other DNA
cgggtggcgaatgggactttgttacccatcaaatccaccggttttagagctagaaatagc

SEQ ID NO: 45 - DNA, 60 bp, synthetic construct
  source 1..60: mol_type = other DNA
accaagacactcaaaaaaaattccaaaagctattaggtaactatgaagggtctatcctca

SEQ ID NO: 46 - DNA, 60 bp, synthetic construct
  source 1..60: mol_type = other DNA
acacggagagtgataccttatcatctgctgcgctaagagtcatcaatccattagagatgg

SEQ ID NO: 47 - DNA, 60 bp, synthetic construct
  source 1..60: mol_type = other DNA
accaagacactcaaaaaaaattccaaaagctattaggtaactatgaagggcttatcctca

SEQ ID NO: 48 - DNA, 60 bp, synthetic construct
  source 1..60: mol_type = other DNA
acacggagagtgataccttatcatctgctgcgctaagagtcatcataaattcgtaataga

SEQ ID NO: 49 - DNA, 60 bp, synthetic construct
  source 1..60: mol_type = other DNA
accaagacactcaaaaaaaattccaaaagctattaggtaactatgaagaatatcatttcg

SEQ ID NO: 50 - DNA, 23 bp, synthetic construct
  source 1..23: mol_type = other DNA
cattttcgacactccgaagaagg

SEQ ID NO: 51 - DNA, 19 bp, synthetic construct
  source 1..19: mol_type = other DNA
ggtatttaatatatacacg

SEQ ID NO: 52 - DNA, 20 bp, synthetic construct
  source 1..20: mol_type = other DNA
caaggccatgtcttctcgtt

SEQ ID NO: 53 - DNA, 20 bp, synthetic construct
  source 1..20: mol_type = other DNA
gtttctggccgagctacaag

SEQ ID NO: 54 - DNA, 21 bp, synthetic construct
  source 1..21: mol_type = other DNA
caaaacccctacaaaaactgc

SEQ ID NO: 55 - DNA, 40 bp, synthetic construct
  source 1..40: mol_type = other DNA
ccactagcctttaagcatgcgggtacgggtttgttgaagt

SEQ ID NO: 56 - DNA, 40 bp, synthetic construct
  source 1..40: mol_type = other DNA
acttcaacaaacccgtacccgcatgcttaaaggctagtgg

SEQ ID NO: 57 - DNA, 20 bp, synthetic construct
  source 1..20: mol_type = other DNA
cttgatggtttcttctctcc

SEQ ID NO: 58 - DNA, 60 bp, synthetic construct
  source 1..60: mol_type = other DNA
acacggagagtgataccttatcatctgctgcgctaagagtcattataatgcctgctgact

SEQ ID NO: 59 - DNA, 24 bp, synthetic construct
  source 1..24: mol_type = other DNA
atccgttcaaattataaaagtcac

SEQ ID NO: 60 - DNA, 27 bp, synthetic construct
  source 1..27: mol_type = other DNA
acagttcataaataaacgtatgagatc

SEQ ID NO: 61 - DNA, 20 bp, synthetic construct
  source 1..20: mol_type = other DNA
cggaatatgggactacttcg

SEQ ID NO: 62 - DNA, 24 bp, synthetic construct
  source 1..24: mol_type = other DNA
cttgaacatgtgtgttctttgtgg

SEQ ID NO: 63 - DNA, 24 bp, synthetic construct
  source 1..24: mol_type = other DNA
taacgattgcccatgaactcgacc

SEQ ID NO: 64 - DNA, 24 bp, synthetic construct
  source 1..24: mol_type = other DNA
tctgattgcattatcgttgtctcc

SEQ ID NO: 65 - DNA, 60 bp, synthetic construct
  source 1..60: mol_type = other DNA
ctcacggtgccaaaatgggaagtggcgctcttctaatggtggtcgccttcttttacaacc

SEQ ID NO: 66 - DNA, 60 bp, synthetic construct
  source 1..60: mol_type = other DNA
agacaacgggagcaatccccaggttgtaaaagaaggcgaccaccattagaagagcgccac

SEQ ID NO: 67 - DNA, 60 bp, synthetic construct
  source 1..60: mol_type = other DNA
aaatcaattattctggctcgtaacgcctataatatggcaaatatcgtagtcgctgttttg

SEQ ID NO: 68 - DNA, 60 bp, synthetic construct
  source 1..60: mol_type = other DNA
gcaaaagacaacgggagcaatccccaggttgtaaaagaacgcaacgaccattagaagagc

SEQ ID NO: 69 - DNA, 60 bp, synthetic construct
  source 1..60: mol_type = other DNA
caaaatgggaagtggcgctcttctaatggtcgttgcgttcttttacaacctggggattgc

SEQ ID NO: 70 - DNA, 60 bp, synthetic construct
  source 1..60: mol_type = other DNA
cagttggtacaaaatcaaaacagcgactacgatatttgccatattataggcgttacgagc

SEQ ID NO: 71 - DNA, 45 bp, synthetic construct
  source 1..45: mol_type = other DNA
gctcgtaatgcgtacaacatggcatgtatcgtagtcgctgttttg

SEQ ID NO: 72 - DNA, 45 bp, synthetic construct
  source 1..45: mol_type = other DNA
caaaacagcgactacgatacatgccatgttgtacgcattacgagc

SEQ ID NO: 73 - DNA, 60 bp, synthetic construct
  source 1..60: mol_type = other DNA
gcgtacaacataggatgtatcgtaactgctgtcctgacattgtaccaactgaactctgag

SEQ ID NO: 74 - DNA, 60 bp, synthetic construct
  source 1..60: mol_type = other DNA
tcagttggtacaatgtcaggacagcagttacgatacatcctatgttgtacgcattacgag

SEQ ID NO: 75 - DNA, 22 bp, synthetic construct
  source 1..22: mol_type = other DNA
gtcgttgttggtttccacaacg

SEQ ID NO: 76 - DNA, 22 bp, synthetic construct
  source 1..22: mol_type = other DNA
cgttgtggaaaccaacaacgac

SEQ ID NO: 77 - DNA, 22 bp, synthetic construct
  source 1..22: mol_type = other DNA
gaagctgtcagacgatgaaggc

SEQ ID NO: 78 - DNA, 25 bp, synthetic construct
  source 1..25: mol_type = other DNA
gacttcgacgatcttagtagcagcg

SEQ ID NO: 79 - DNA, 60 bp, synthetic construct
  source 1..60: mol_type = other DNA
gcagcacgtctggagcgtacagatcttactgcgcaacatataaagaggcgtgatatgctc

SEQ ID NO: 80 - DNA, 60 bp, synthetic construct
  source 1..60: mol_type = other DNA
ttacataaaaccaacgaaataccttaaccaagtaataaataataaagctaatgtgctagg

SEQ ID NO: 81 - DNA, 22 bp, synthetic construct
  source 1..22: mol_type = other DNA
taatacgtactcagagacacgc

SEQ ID NO: 82 - DNA, 23 bp, synthetic construct
  source 1..23: mol_type = other DNA
actttacggaggaataaagagcc

SEQ ID NO: 83 - DNA, 60 bp, synthetic construct
  source 1..60: mol_type = other DNA
aatgggaagtggcgctcttctaatggtggtcgccttctcatacaacctggggattgctcc

SEQ ID NO: 84 - DNA, 60 bp, synthetic construct
  source 1..60: mol_type = other DNA
gcaaaagacaacgggagcaatccccaggttgtatgagaaggcgaccaccattagaagagc

SEQ ID NO: 85 - DNA, 60 bp, synthetic construct
  source 1..60: mol_type = other DNA
ctaaatcaattattctggctcgtaacgcctataatatagcatgtatcgtaactgctgtcc

SEQ ID NO: 86 - DNA, 60 bp, synthetic construct
  source 1..60: mol_type = other DNA
ttggtacaatgtcaggacagcagttacgatacatgctatattataggcgttacgagccag

SEQ ID NO: 87 - DNA, 60 bp, synthetic construct
  source 1..60: mol_type = other DNA
aaatcaattattctggctcgtaacgcctataatatgggatgtatcgtaactgctgtcctg

SEQ ID NO: 88 - DNA, 60 bp, synthetic construct
  source 1..60: mol_type = other DNA
ttggtacaatgtcaggacagcagttacgatacatcccatattataggcgttacgagccag

SEQ ID NO: 89 - DNA, 60 bp, synthetic construct
  source 1..60: mol_type = other DNA
atcaattattctggctcgtaacgcctataatatggcaaatatcgtaactgctgtcctgac

SEQ ID NO: 90 - DNA, 60 bp, synthetic construct
  source 1..60: mol_type = other DNA
cagttggtacaatgtcaggacagcagttacgatatttgccatattataggcgttacgagc

SEQ ID NO: 91 - DNA, 60 bp, synthetic construct
  source 1..60: mol_type = other DNA
gctcgtaacgcctataatatggcatgtatcgtagtcgctgtcctgacattgtaccaactg

SEQ ID NO: 92 - DNA, 60 bp, synthetic construct
  source 1..60: mol_type = other DNA
tcagagttcagttggtacaatgtcaggacagcgactacgatacatgccatattataggcg

SEQ ID NO: 93 - DNA, 60 bp, synthetic construct
  source 1..60: mol_type = other DNA
gcctataatatggcatgtatcgtaactgctgtcctgattttgtaccaactgaactctgag

SEQ ID NO: 94 - DNA, 60 bp, synthetic construct
  source 1..60: mol_type = other DNA
ccagttccatttctcagagttcagttggtacaaaatcaggacagcagttacgatacatgc

SEQ ID NO: 95 - DNA, 60 bp, synthetic construct
  source 1..60: mol_type = other DNA
gctaaaaagaacaacgccacaatcaatgtgtaacgattacccatcaaatccaccgaggga

SEQ ID NO: 96 - DNA, 60 bp, synthetic construct
  source 1..60: mol_type = other DNA
ggtttacagttgacaggtccctcggtggatttgatgggtaatcgttacacattgattgtg

SEQ ID NO: 97 - DNA, 60 bp, synthetic construct
  source 1..60: mol_type = other DNA
acagcccaatctgccaagataccgaaatctcccattctcctgtcttgctattcagagaac

SEQ ID NO: 98 - DNA, 60 bp, synthetic construct
  source 1..60: mol_type = other DNA
cagaaggaatatggttctctgaatagcaagacaggagaatgggagatttcggtatcttgg

SEQ ID NO: 99 - DNA, 60 bp, synthetic construct
  source 1..60: mol_type = other DNA
aataagttgtcaggtagtatcttaaagccaaaggacagatttcagaggcataagacacag

SEQ ID NO: 100 - DNA, 60 bp, synthetic construct
  source 1..60: mol_type = other DNA
ttccaatgtctgactgtgtcttatgcctctgaaatctgtcctttggctttaagatactac

SEQ ID NO: 101 - DNA, 60 bp, synthetic construct
  source 1..60: mol_type = other DNA
gaataatactaaaagtgaatgctgtttcagtgctaacaccggctttctcgtagaagtaag

SEQ ID NO: 102 - DNA, 60 bp, synthetic construct
  source 1..60: mol_type = other DNA
ctaataggttactcaacttacttctacgagaaagccggtgttagcactgaaacagcattc

SEQ ID NO: 103 - DNA, 60 bp, synthetic construct
  source 1..60: mol_type = other DNA
tagtcttcctttcttgactaaccaccatggggattcaggtgcaaagaatatccctattgc

SEQ ID NO: 104 - DNA, 60 bp, synthetic construct
  source 1..60: mol_type = other DNA
tggcctcttcccctggcaatagggatattctttgcacctgaatccccatggtggttagtc

SEQ ID NO: 105 - DNA, 60 bp, synthetic construct
  source 1..60: mol_type = other DNA
cggagagtgataccttatcatctgctgcgctaagagtcatcataaattcgtaatagatgg

SEQ ID NO: 106 - DNA, 60 bp, synthetic construct
  source 1..60: mol_type = other DNA
atgtcaaaacagcagttacgatacatcccatgttgtacgcattacgagctaaaatgatcg

SEQ ID NO: 107 - DNA, 60 bp, synthetic construct
  source 1..60: mol_type = other DNA
gcgtacaacatgggatgtatcgtaactgctgttttgacattgtaccaactgaactctgag

SEQ ID NO: 108 - DNA, 60 bp, synthetic construct
  source 1..60: mol_type = other DNA
atgtcaaaacagcgactacgatacatcccatgttgtacgcattacgagctaaaatgatcg

SEQ ID NO: 109 - DNA, 60 bp, synthetic construct
  source 1..60: mol_type = other DNA
gcgtacaacatgggatgtatcgtagtcgctgttttgacattgtaccaactgaactctgag

SEQ ID NO: 110 - DNA, 60 bp, synthetic construct
  source 1..60: mol_type = other DNA
ataaacaagctattcttgttcttctccggttgatacagtccctcagacaatccaaatagg

SEQ ID NO: 111 - DNA, 60 bp, synthetic construct
  source 1..60: mol_type = other DNA
aagctgtcagacgatgaaggctcctatttggattgtctgagggactgtatcaaccggaga

SEQ ID NO: 112 - DNA, 60 bp, synthetic construct
  source 1..60: mol_type = other DNA
ctgttccattctccctttcttgatcaaccaccatggagattcaggtgcaaagaatatcgc

SEQ ID NO: 113 - DNA, 60 bp, synthetic construct
  source 1..60: mol_type = other DNA
atctggcctgttcctctagcaatagcgatattctttgcacctgaatctccatggtggttg

SEQ ID NO: 114 - DNA, 60 bp, synthetic construct
  source 1..60: mol_type = other DNA
gcaaatggtagcttatatcccaaatccgagtctgggtatttgttttgggaatttttcatg

SEQ ID NO: 115 - DNA, 60 bp, synthetic construct
  source 1..60: mol_type = other DNA
cagctttttgctgcaggcatcatgaaaaattcccaaaacaaatacccagactcggatttg

SEQ ID NO: 116 - DNA, 60 bp, synthetic construct
  source 1..60: mol_type = other DNA
gattcgaatatgtcgtcaggtagtacctcagcgccataggacagatttcggaagcatatg

SEQ ID NO: 117 - DNA, 60 bp, synthetic construct
  source 1..60: mol_type = other DNA
gtttccaatgtttgacagtctcatatgcttccgaaatctgtcctatggcgctgaggtact

SEQ ID NO: 118 - DNA, 60 bp, synthetic construct
  source 1..60: mol_type = other DNA
cattctccctttcttgatcaaccaccatggagattcaggtgcaaagaatatcgctattgc

SEQ ID NO: 119 - DNA, 22 bp, synthetic construct
  source 1..22: mol_type = other DNA
ccatgtaatctacaaaaggacc

SEQ ID NO: 120 - DNA, 22 bp, synthetic construct
  source 1..22: mol_type = other DNA
gaccaatccaacataaacaagc

SEQ ID NO: 121 - DNA, 21 bp, synthetic construct
  source 1..21: mol_type = other DNA
ccaaattaaaacagccaggca

SEQ ID NO: 122 - DNA, 22 bp, synthetic construct
  source 1..22: mol_type = other DNA
gactcggacttaggatatacgc

SEQ ID NO: 123 - DNA, 25 bp, synthetic construct
  source 1..25: mol_type = other DNA
gaccgttatactgttcattataggc

SEQ ID NO: 124 - DNA, 22 bp, synthetic construct
  source 1..22: mol_type = other DNA
ataccgaaatctcccattctcc

SEQ ID NO: 125 - DNA, 20 bp, synthetic construct
  source 1..20: mol_type = other DNA
tttgcctgctctagtcttcc

SEQ ID NO: 126 - DNA, 21 bp, synthetic construct
  source 1..21: mol_type = other DNA
gaaaacgtatcccaaagctgc

SEQ ID NO: 127 - DNA, 24 bp, synthetic construct
  source 1..24: mol_type = other DNA
cctttggctttaagatactacctg

SEQ ID NO: 128 - DNA, 21 bp, synthetic construct
  source 1..21: mol_type = other DNA
gtcgccttcttttacaacctg

SEQ ID NO: 129 - DNA, 20 bp, synthetic construct
  source 1..20: mol_type = other DNA
gaagaagcctgactttgcac

SEQ ID NO: 130 - DNA, 22 bp, synthetic construct
  source 1..22: mol_type = other DNA
ggtagcttatatcccaaatccg

SEQ ID NO: 131 - DNA, 20 bp, synthetic construct
  source 1..20: mol_type = other DNA
agaactgctggtgagtatgg

SEQ ID NO: 132 - DNA, 60 bp, synthetic construct
  source 1..60: mol_type = other DNA
cggctaaaaagaacaacgccacaatcaatgtgtaacgattacccatcaaatccaccgagg

SEQ ID NO: 133 - DNA, 60 bp, synthetic construct
  source 1..60: mol_type = other DNA
gtttacagttgacaggtccctcggtggatttgatgggtaatcgttacacattgattgtgg

SEQ ID NO: 134 - DNA, 60 bp, synthetic construct; identical to SEQ ID NO: 103
  source 1..60: mol_type = other DNA

SEQ ID NO: 135 - DNA, 60 bp, synthetic construct
  source 1..60: mol_type = other DNA
gatttggcctcttcccctggcaatagggatattctttgcacctgaatccccatggtggtt

SEQ ID NO: 136 - DNA, 60 bp, synthetic construct
  source 1..60: mol_type = other DNA
tttgaccggcccagcacagacaggctattctcgttctcctccggttgatacagtccttca

SEQ ID NO: 137 - DNA, 60 bp, synthetic construct
  source 1..60: mol_type = other DNA
ggtcatactgggattgtgtgaaggactgtatcaaccggaggagaacgagaatagcctgtc

SEQ ID NO: 138 - DNA, 60 bp, synthetic construct; identical to SEQ ID NO: 101
  source 1..60: mol_type = other DNA

SEQ ID NO: 139 - DNA, 60 bp, synthetic construct
  source 1..60: mol_type = other DNA
actaataggttactcaacttacttctacgagaaagccggtgttagcactgaaacagcatt

SEQ ID NO: 140 - DNA, 60 bp, synthetic construct
  source 1..60: mol_type = other DNA
tgtctgtgctgggccggtcaaactgtttgtggtacacaactaataggttactcaacttac

SEQ ID NO: 141 - DNA, 60 bp, synthetic construct
  source 1..60: mol_type = other DNA
gctttctcgtagaagtaagttgagtaacctattagttgtgtaccacaaacagtttgaccg

SEQ ID NO: 142 - DNA, 60 bp, synthetic construct
  source 1..60: mol_type = other DNA
ttgtttatgttggattggtcaaaccacttgtggtgcgtcactgataggatattcaaccta

SEQ ID NO: 143 - DNA, 60 bp, synthetic construct
  source 1..60: mol_type = other DNA
gccttttcgtaaaaataggttgaatatcctatcagtgacgcaccacaagtggtttgacca

SEQ ID NO: 144 - DNA, 60 bp, synthetic construct
  source 1..60: mol_type = other DNA
tttacaaatgactggtccttttgtagattacatgggtaaccgctacacattgattatggc

SEQ ID NO: 145 - DNA, 60 bp, synthetic construct
  source 1..60: mol_type = other DNA
agttaagaacatcaacgccataatcaatgtgtagcggttacccatgtaatctacaaaagg

SEQ ID NO: 146 - DNA, 60 bp, synthetic construct
  source 1..60: mol_type = other DNA
tgtttccaatgtttgacagtctcatatgcttccgaaatctgtcctatggcgctgaggtac

SEQ ID NO: 147 - DNA, 60 bp, synthetic construct
  source 1..60: mol_type = other DNA
cgaatatgtcgtcaggtagtacctcagcgccataggacagatttcggaagcatatgagac

SEQ ID NO: 148 - DNA, 60 bp, synthetic construct
  source 1..60: mol_type = other DNA
tgtttccaatgtctgactgtgtcttatgcctctgaaatctgtcctttggctttaagatac

SEQ ID NO: 149 - DNA, 60 bp, synthetic construct; identical to SEQ ID NO: 99
  source 1..60: mol_type = other DNA

SEQ ID NO: 150 - DNA, 60 bp, synthetic construct
  source 1..60: mol_type = other DNA
agcctgtctgtgctgggccggtcaaactgtttgtggtacatcactaataggttactcaac

SEQ ID NO: 151 - DNA, 60 bp, synthetic construct
  source 1..60: mol_type = other DNA
ctttctcgtagaagtaagttgagtaacctattagtgatgtaccacaaacagtttgaccgg

SEQ ID NO: 152 - DNA, 60 bp, synthetic construct
  source 1..60: mol_type = other DNA
tgtctgtgctgggccggtcaaactgtttgtggtgcgcaactaataggttactcaacttac

SEQ ID NO: 153 - DNA, 60 bp, synthetic construct
  source 1..60: mol_type = other DNA
accggctttctcgtagaagtaagttgagtaacctattagttgcgcaccacaaacagtttg

SEQ ID NO: 154 - DNA, 60 bp, synthetic construct
  source 1..60: mol_type = other DNA
ctgtgctgggccggtcaaactgtttgtggtacatcactaataggttactcaacttacttc

SEQ ID NO: 155 - DNA, 60 bp, synthetic construct; identical to SEQ ID NO: 151
  source 1..60: mol_type = other DNA

SEQ ID NO: 156 - DNA, 60 bp, synthetic construct
  source 1..60: mol_type = other DNA
gctgggccggtcaaactgtttgtggtgcgcaactaataggttactcaacttacttctacg

SEQ ID NO: 157 - DNA, 60 bp, synthetic construct
  source 1..60: mol_type = other DNA
ggctttctcgtagaagtaagttgagtaacctattagttgcgcaccacaaacagtttgacc

SEQ ID NO: 158 - DNA, 24 bp, synthetic construct
  source 1..24: mol_type = other DNA
ccttcactcatctctccatacacg

SEQ ID NO: 159 - DNA, 25 bp, synthetic construct
  source 1..25: mol_type = other DNA
ctgaatgatctggtgaactttacgg

SEQ ID NO: 160 - DNA, 60 bp, synthetic construct
  source 1..60: mol_type = other DNA
atcaattattctggctcgtaacgcctataatatggcacaaatcgtaactgctgtcctgac

SEQ ID NO: 161 - DNA, 60 bp, synthetic construct
  source 1..60: mol_type = other DNA
tcagttggtacaatgtcaggacagcagttacgatttgtgccatattataggcgttacgag

SEQ ID NO: 162 - DNA, 60 bp, synthetic construct
  source 1..60: mol_type = other DNA
atcaattattctggctcgtaacgcctataatatggcagatatcgtaactgctgtcctgac

SEQ ID NO: 163 - DNA, 60 bp, synthetic construct
  source 1..60: mol_type = other DNA
tcagttggtacaatgtcaggacagcagttacgatatctgccatattataggcgttacgag

SEQ ID NO: 164 - DNA, 60 bp, synthetic construct
  source 1..60: mol_type = other DNA
gcctataatatggcatgtatcgtaactgctgtcctggttttgtaccaactgaactctgag

SEQ ID NO: 165 - DNA, 60 bp, synthetic construct
  source 1..60: mol_type = other DNA
cccagttccatttctcagagttcagttggtacaaaaccaggacagcagttacgatacatg

SEQ ID NO: 166 - DNA, 60 bp, synthetic construct
  source 1..60: mol_type = other DNA
gctgggccggtcaaactgtttgtggtgcgtgtctaataggttactcaacttacttctacg

SEQ ID NO: 167 - DNA, 60 bp, synthetic construct
  source 1..60: mol_type = other DNA
ggctttctcgtagaagtaagttgagtaacctattagacacgcaccacaaacagtttgacc

SEQ ID NO: 168 - DNA, 60 bp, synthetic construct
  source 1..60: mol_type = other DNA
gctgggccggtcaaactgtttgtggtgcggaactaataggttactcaacttacttctacg

SEQ ID NO: 169 - DNA, 60 bp, synthetic construct
  source 1..60: mol_type = other DNA
ggctttctcgtagaagtaagttgagtaacctattagttccgcaccacaaacagtttgacc

SEQ ID NO: 170 - DNA, 60 bp, synthetic construct
  source 1..60: mol_type = other DNA
atcaattattctggctcgtaacgcctataatatggcatttatcgtaactgctgtcctgac

SEQ ID NO: 171 - DNA, 60 bp, synthetic construct
  source 1..60: mol_type = other DNA
atcaattattctggctcgtaacgcctataatatggcattgatcgtaactgctgtcctgac

SEQ ID NO: 172 - DNA, 60 bp, synthetic construct
  source 1..60: mol_type = other DNA
atcaattattctggctcgtaacgcctataatatggcatatatcgtaactgctgtcctgac

SEQ ID NO: 173 - DNA, 60 bp, synthetic construct
  source 1..60: mol_type = other DNA
atcaattattctggctcgtaacgcctataatatggcacatatcgtaactgctgtcctgac

SEQ ID NO: 174 - DNA, 60 bp, synthetic construct
  source 1..60: mol_type = other DNA
atcaattattctggctcgtaacgcctataatatggcaattatcgtaactgctgtcctgac

SEQ ID NO: 175 - DNA, 60 bp, synthetic construct
  source 1..60: mol_type = other DNA
atcaattattctggctcgtaacgcctataatatggcaatgatcgtaactgctgtcctgac

SEQ ID NO: 176 - DNA, 60 bp, synthetic construct
  source 1..60: mol_type = other DNA
atcaattattctggctcgtaacgcctataatatggcaaaaatcgtaactgctgtcctgac

SEQ ID NO: 177 - DNA, 60 bp, synthetic construct
  source 1..60: mol_type = other DNA
atcaattattctggctcgtaacgcctataatatggcagttatcgtaactgctgtcctgac

SEQ ID NO: 178 - DNA, 60 bp, synthetic construct
  source 1..60: mol_type = other DNA
atcaattattctggctcgtaacgcctataatatggcagaaatcgtaactgctgtcctgac

SEQ ID NO: 179 - DNA, 60 bp, synthetic construct
  source 1..60: mol_type = other DNA
atcaattattctggctcgtaacgcctataatatggcatctatcgtaactgctgtcctgac

SEQ ID NO: 180 - DNA, 60 bp, synthetic construct
  source 1..60: mol_type = other DNA
atcaattattctggctcgtaacgcctataatatggcatggatcgtaactgctgtcctgac

SEQ ID NO: 181 - DNA, 60 bp, synthetic construct
  source 1..60: mol_type = other DNA
atcaattattctggctcgtaacgcctataatatggcaccaatcgtaactgctgtcctgac

SEQ ID NO: 182 - DNA, 60 bp, synthetic construct
  source 1..60: mol_type = other DNA
atcaattattctggctcgtaacgcctataatatggcaagaatcgtaactgctgtcctgac

SEQ ID NO: 183 - DNA, 60 bp, synthetic construct
  source 1..60: mol_type = other DNA
atcaattattctggctcgtaacgcctataatatggcaactatcgtaactgctgtcctgac

SEQ ID NO: 184 - DNA, 60 bp, synthetic construct
  source 1..60: mol_type = other DNA
atcaattattctggctcgtaacgcctataatatggcagctatcgtaactgctgtcctgac

SEQ ID NO: 185 - DNA, 60 bp, synthetic construct
  source 1..60: mol_type = other DNA
atcaattattctggctcgtaacgcctataatatggcaggtatcgtaactgctgtcctgac

SEQ ID NO: 186 - DNA, 24 bp, synthetic construct
  source 1..24: mol_type = other DNA
catattataggcgttacgagccag

SEQ ID NO: 187 - DNA, 23 bp, synthetic construct
  source 1..23: mol_type = other DNA
tgccatattataggcgttacgag

SEQ ID NO: 188 - DNA, 57 bp, synthetic construct
  source 1..57: mol_type = other DNA
agcaatccccaggttgtaaaagaaggcgaccaccattagaagagcgccacttcccat

SEQ ID NO: 189 - DNA, 57 bp, synthetic construct
  source 1..57: mol_type = other DNA
atgggaagtggcgctcttctaatggtggtcgccttcttttacaacctggggattgct

SEQ ID NO: 190 - DNA, 55 bp, synthetic construct
  source 1..55: mol_type = other DNA
gggactatgttttgcgacgttagtatgggctgtctttgatctgcccgaaaccgca

SEQ ID NO: 191 - DNA, 55 bp, synthetic construct
  source 1..55: mol_type = other DNA
tgcggtttcgggcagatcaaagacagcccatactaacgtcgcaaaacatagtccc